SEQUENCE LISTING (124 sequences)

SEQ ID NO: 1 - protein, 11 aa, synthetic construct
QASQSIGSSLA

SEQ ID NO: 2 - protein, 7 aa, synthetic construct
QASKLAS

SEQ ID NO: 3 - protein, 12 aa, synthetic construct
QCTGNGGDFIGA

SEQ ID NO: 4 - protein, 5 aa, synthetic construct
SYYVR

SEQ ID NO: 5 - protein, 16 aa, synthetic construct
IISDSGSTYYASWAKG

SEQ ID NO: 6 - protein, 10 aa, synthetic construct
GSGYTIPTDL

SEQ ID NO: 7 - protein, 80 aa, synthetic construct
DPVMTQTPASVSEPVGGTVTIKCWYQQKPGQRPKLLIYGVPSRFKGSRSGTEFTLTISDLECADAATYYCFGGGTEVVVK

SEQ ID NO: 8 - protein, 84 aa, synthetic construct
QSLEESGGGLVTPGTPLTLTCTASGFSLSWVRQAPGKGLEYIGRFTISKTSTTVDLKFTSPTTEDTATYFCARWGPGTLVTVSS

SEQ ID NO: 9 - protein, 217 aa, synthetic construct
DPVMTQTPASVSEPVGGTVTIKCQASQSIGSSLAWYQQKPGQRPKLLIYQASKLASGVPSRFKGSRSGTEFTLTISDLECADAATYYCQCTGNGGDFIGAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 445 aa, synthetic construct
QSLEESGGGLVTPGTPLTLTCTASGFSLSSYYVRWVRQAPGKGLEYIGIISDSGSTYYASWAKGRFTISKTSTTVDLKFTSPTTEDTATYFCARGSGYTIPTDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 16 aa, synthetic construct
RSSESLVYSNGKSYLS

SEQ ID NO: 12 - protein, 7 aa, synthetic construct
EVSKRDS

SEQ ID NO: 13 - protein, 10 aa, synthetic construct
MQGTYWPPIT

SEQ ID NO: 14 - protein, 5 aa, synthetic construct
LHYMH

SEQ ID NO: 15 - protein, 17 aa, synthetic construct
RISNDGSEGWYAESVKG

SEQ ID NO: 16 - protein, 10 aa, synthetic construct
DPDTSNKIDY

SEQ ID NO: 17 - protein, 80 aa, synthetic construct
DVVMTQSPLSLPVTLGQPASISCWFQQRPGQSPRRLLYGVPDKFSGSGSGTDFTLKISRVEAEDVGVYYCFGQGTRLEIK

SEQ ID NO: 18 - protein, 80 aa, synthetic construct; identical to SEQ ID NO: 17

SEQ ID NO: 19 - protein, 220 aa, synthetic construct
DVVMTQSPLSLPVTLGQPASISCRSSESLVYSNGKSYLSWFQQRPGQSPRRLLYEVSKRDSGVPDKFSGSGSGTDFTLKISRVEAEDVGVYYCMQGTYWPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 449 aa, synthetic construct
QVQLVESGGDVVQPGGSLRLSCAASGFTFNLHYMHWVRQAPGRGLEWVSRISNDGSEGWYAESVKGRFTISRDNSKNSLYLQMNSLRAEDTAVYYCARDPDTSNKIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 11 aa, synthetic construct
QASQNTDIRLA

SEQ ID NO: 22 - protein, 7 aa, synthetic construct
SASTLAS

SEQ ID NO: 23 - protein, 21 aa, synthetic construct
DDAATYYCLGNYDCSYADCYA

SEQ ID NO: 24 - protein, 5 aa, synthetic construct
NYEMG

SEQ ID NO: 25 - protein, 16 aa, synthetic construct
YIRTGGSAYYASWAKG

SEQ ID NO: 26 - protein, 11 aa, synthetic construct
PYAFVSLINDL

SEQ ID NO: 27 - protein, 72 aa, synthetic construct
AQVLTQTPSSVSAAVGGTVTINCWYQQKPGQPPKRLIYGVPSRFKGSGSGTQFTLTISDLECFGGGTEVVVR

SEQ ID NO: 28 - protein, 84 aa, synthetic construct
QSLEESGGRLVTPGTPLTLTCTVSGIDLNWVRQAPGKGLEWIGRFTISKTSTTVDLKMTSLATEDTATYFCARWGPGTLVTVSS

SEQ ID NO: 29 - protein, 218 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 446 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 13 aa, synthetic construct
QASQSVWKNNYLS

SEQ ID NO: 32 - protein, 7 aa, synthetic construct
TASSLAS

SEQ ID NO: 33 - protein, 10 aa, synthetic construct
AGDVGGGIRT

SEQ ID NO: 34 - protein, 5 aa, synthetic construct
SYTTS

SEQ ID NO: 35 - protein, 17 aa, synthetic construct
VIDVGSDDTYYATWAKG

SEQ ID NO: 36 - protein, 11 aa, synthetic construct
HGATGGTVFDL

SEQ ID NO: 37 - protein, 80 aa, synthetic construct
AQVLTQTPSPVSAAVGGTVTINCWFQQKPGQPPKLLIYGVPSRFKGSGSGTQFTLTISDLECDDAATYYCFGGGTEVVVK

SEQ ID NO: 38 - protein, 84 aa, synthetic construct
QSLEESGGRLVTPGTPLTLTCTASGFSLSWVRQAPGKGLEWVGRFTISRTSTTVDLKMTSLTAADTATYFCTRWGPGTLVTVSS

SEQ ID NO: 39 - protein, 217 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 447 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 14 aa, synthetic construct
TGTSSDVGGYNYVS

SEQ ID NO: 42 - protein, 7 aa, synthetic construct
EVSKRPS

SEQ ID NO: 43 - protein, 10 aa, synthetic construct
SSHAGSKNVI

SEQ ID NO: 44 - protein, 5 aa, synthetic construct
GHYMS

SEQ ID NO: 45 - protein, 17 aa, synthetic construct
KMNQDGSSRSYVDSVKG

SEQ ID NO: 46 - protein, 8 aa, synthetic construct
EEWYRFDY

SEQ ID NO: 47 - protein, 110 aa, synthetic construct
QSALTQPPSASGSPGQSVTISCTGTSSDVGGYNYVSWYRQHPGKAPKLMIYEVSKRPSGVPDRFSGSKSGNTASLTVSGLQADDEGDYYCSSHAGSKNVIFGGGTKVTVL

SEQ ID NO: 48 - protein, 110 aa, synthetic construct
EVQLVESGGGLVQPGGSLRLSCAASGFAFSGHYMSWVRQAPGKGLEWVAKRSYVDSVKGRFTISRDNAKNSLYLQMNSLRAEDTAVYYCTKEEWYRFDYWGQGTLVTVSS

SEQ ID NO: 49 - protein, 216 aa, synthetic construct
QSALTQPPSASGSPGQSVTISCTGTSSDVGGYNYVSWYRQHPGKAPKLMIYEVSKRPSGVPDRFSGSKSGNTASLTVSGLQADDEGDYYCSSHAGSKNVIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 447 aa, synthetic construct
EVQLVESGGGLVQPGGSLRLSCAASGFAFSGHYMSWVRQAPGKGLEWVAKMNQDGSSRSYVDSVKGRFTISRDNAKNSLYLQMNSLRAEDTAVYYCTKEEWYRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 13 aa, synthetic construct
TGNSNNVGNQGAA

SEQ ID NO: 52 - protein, 7 aa, synthetic construct
RNNNRPS

SEQ ID NO: 53 - protein, 11 aa, synthetic construct
SAWDSSLNAWV

SEQ ID NO: 54 - protein, 5 aa, synthetic construct
NYVMH

SEQ ID NO: 55 - protein, 16 aa, synthetic construct
IWSDGSTIFHADSVKG

SEQ ID NO: 56 - protein, 14 aa, synthetic construct
EPRAIADNYYGMDV

SEQ ID NO: 57 - protein, 79 aa, synthetic construct
QAGLTQPPSVSKGLRQTATLTCWLQQHQGHPPKLLSYGISERLSASRSGNTASLTITGLQPEDEADYYCFGGGTKLAVL

SEQ ID NO: 58 - protein, 88 aa, synthetic construct
QVQLVESGGDVVQPGRSLRLSCAASGFTFSWVRQAPGEGLEWVSLRFTISRDNSKNTLYLQMDSLRAEDTAVYYCARWGQGTSVTVSS

SEQ ID NO: 59 - protein, 216 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 60 - protein, 453 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 13 aa, synthetic construct
QASKTVYDDNALA

SEQ ID NO: 62 - protein, 7 aa, synthetic construct
KASTLAS

SEQ ID NO: 63 - protein, 12 aa, synthetic construct
AGGYIYDSGDHA

SEQ ID NO: 64 - protein, 5 aa, synthetic construct
RGQVG

SEQ ID NO: 65 - protein, 16 aa, synthetic construct
FKGYGGNAFYTNWAKG

SEQ ID NO: 66 - protein, 10 aa, synthetic construct
VAGDINHLDL

SEQ ID NO: 67 - protein, 79 aa, synthetic construct
AAVLTQTPSPVSAAVGGTVSINCWYQQKPGQPPKLLIYGVPSRFSGSGSGTQFTLTISDVQCDDAATYYCFGGGTEVVV

SEQ ID NO: 68 - protein, 85 aa, synthetic construct
QSLEESGGRLVTPGTPLTLTCTVSGIDLSWVRQAPGEGLEYIGRFTISKTSSTTVDLKITTPTTEDTATYFCARWGQGTLVTVSS

SEQ ID NO: 69 - protein, 219 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 446 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 11 aa, synthetic construct
QASQSIGSYLA

SEQ ID NO: 72 - protein, 7 aa, synthetic construct
YVFKVAP

SEQ ID NO: 73 - protein, 10 aa, synthetic construct
ASYAGSSSSA

SEQ ID NO: 74 - protein, 5 aa, synthetic construct
IYAVG

SEQ ID NO: 75 - protein, 16 aa, synthetic construct
TISTVDRSYYATWAKG

SEQ ID NO: 76 - protein, 13 aa, synthetic construct
GLSCSNTDCAFNI

SEQ ID NO: 77 - protein, 108 aa, synthetic construct
AFELTQTPSSVEAAVGGTVTISCQASQSIGSYLAWYQQKPGQPPKLLIYYVFKVAPGVPSRFSGSGSGTQFTLTISDLECADAATYYCASYAGSSSSAFGGGTEVVVK

SEQ ID NO: 78 - protein, 117 aa, synthetic construct
QSVEESGGRLVTPGTPLTLTCTVSGIDLSIYAVGWVRQAPGKGLEYIGTISTVDRSYYATWAKGRFTISKTSTTVDLKITSPTTEDTATYFCGRGLSCSNTDCAFNIWGPGTLVTVS

SEQ ID NO: 79 - protein, 215 aa, synthetic construct
AFELTQTPSSVEAAVGGTVTISCQASQSIGSYLAWYQQKPGQPPKLLIYYVFKVAPGVPSRFSGSGSGTQFTLTISDLECADAATYYCASYAGSSSSAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 448 aa, synthetic construct
QSVEESGGRLVTPGTPLTLTCTVSGIDLSIYAVGWVRQAPGKGLEYIGTISTVDRSYYATWAKGRFTISKTSTTVDLKITSPTTEDTATYFCGRGLSCSNTDCAFNIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 112 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIK

SEQ ID NO: 82 - protein, 109 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLT

SEQ ID NO: 83 - protein, 219 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 84 - protein, 436 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 85 - protein, 13 aa, synthetic construct
QASQSVGGNNALS

SEQ ID NO: 86 - protein, 7 aa, synthetic construct
GASTLAS

SEQ ID NO: 87 - protein, 11 aa, synthetic construct
LGGYGGIGDNA

SEQ ID NO: 88 - protein, 5 aa, synthetic construct
TYNIC

SEQ ID NO: 89 - protein, 16 aa, synthetic construct
CINTGGSAFYTTWVKG

SEQ ID NO: 90 - protein, 14 aa, synthetic construct
YVDGTGYWGTRLDL

SEQ ID NO: 91 - protein, 111 aa, synthetic construct
AAVLTQTPSPVSAAVGGTVTISCQASQSVGGNNALSWFQQKPGQPPKLLIYGASTLASGVPSRFSASGSGTQFTLTISDVQCDDAATYYCLGGYGGIGDNAFGGGTEVVVK

SEQ ID NO: 92 - protein, 119 aa, synthetic construct
QSVEESGGRLVTPGTPLTLTCTASGFSLSTYNICWVRQSPGKGLEYIGCINTGGSAFYTTWVKGRFTISKASTTVDLRITSPTTEDTAIYFCSSYVDGTGYWGTRLDLWGQGTLVTVSS

SEQ ID NO: 93 - protein, 218 aa, synthetic construct
AAVLTQTPSPVSAAVGGTVTISCQASQSVGGNNALSWFQQKPGQPPKLLIYGASTLASGVPSRFSASGSGTQFTLTISDVQCDDAATYYCLGGYGGIGDNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 449 aa, synthetic construct
QSVEESGGRLVTPGTPLTLTCTASGFSLSTYNICWVRQSPGKGLEYIGCINTGGSAFYTTWVKGRFTISKASTTVDLRITSPTTEDTAIYFCSSYVDGTGYWGTRLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 13 aa, synthetic construct
QSSQSVYGNNWLA

SEQ ID NO: 96 - protein, 7 aa, synthetic construct
DASELAS

SEQ ID NO: 97 - protein, 11 aa, synthetic construct
QGGFIGSDRHG

SEQ ID NO: 98 - protein, 6 aa, synthetic construct
SYYYMC

SEQ ID NO: 99 - protein, 18 aa, synthetic construct
CIYAGNADSTYYATWAKG

SEQ ID NO: 100 - protein, 17 aa, synthetic construct
NYYAGGTAGYAHSAFDP

SEQ ID NO: 101 - protein, 80 aa, synthetic construct
AAVLTQTPSPVSVAVGGTVTINCWYQQKPGQPPKLLIYGVPSRFSGSGYGTEFTLTISGVQCEDAATYYCFGGGTEVVVK

SEQ ID NO: 102 - protein, 85 aa, synthetic construct
QQLEESGGGLVKPGGTLTLTCKASGIDFSWVRQAPGKGLEWVGRFTMSKTSSTTVTLQMTSLTSADTATYFCARWGPGTPVTVSS

SEQ ID NO: 103 - protein, 218 aa, synthetic construct
AAVLTQTPSPVSVAVGGTVTINCQSSQSVYGNNWLAWYQQKPGQPPKLLIYDASELASGVPSRFSGSGYGTEFTLTISGVQCEDAATYYCQGGFIGSDRHGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 456 aa, synthetic construct
QQLEESGGGLVKPGGTLTLTCKASGIDFSSYYYMCWVRQAPGKGLEWVGCIYAGNADSTYYATWAKGRFTMSKTSSTTVTLQMTSLTSADTATYFCARNYYAGGTAGYAHSAFDPWGPGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 11 aa, synthetic construct
QASESISSWLS

SEQ ID NO: 106 - protein, 7 aa, synthetic construct
RASTLES

SEQ ID NO: 107 - protein, 14 aa, synthetic construct
QSYARINSASYSNL

SEQ ID NO: 108 - protein, 5 aa, synthetic construct
TYVMT

SEQ ID NO: 109 - protein, 16 aa, synthetic construct
VMATDGSAYYATWTKG

SEQ ID NO: 110 - protein, 10 aa, synthetic construct
GSGWESYFNT

SEQ ID NO: 111 - protein, 112 aa, synthetic construct
DIVMTQTPSSASEPVGGTVTIKCQASESISSWLSWYQQKPGQPPKLLIYRASTLESGVPSRFKGSGSGTEFTLTISDLECADAATYFCQSYARINSASYSNLFGGGTEVVVK

SEQ ID NO: 112 - protein, 115 aa, synthetic construct
QSVEESGGRLVTPGTPLTLTCTASGFSLSTYVMTWVRQAPGKGLEWIGVMATDGSAYYATWTKGRLTISRTSTTVELTITSPTTEDTATYFCARGSGWESYFNTWGPGTLVTVSL

SEQ ID NO: 113 - protein, 219 aa, synthetic construct
DIVMTQTPSSASEPVGGTVTIKCQASESISSWLSWYQQKPGQPPKLLIYRASTLESGVPSRFKGSGSGTEFTLTISDLECADAATYFCQSYARINSASYSNLFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 445 aa, synthetic construct
QSVEESGGRLVTPGTPLTLTCTASGFSLSTYVMTWVRQAPGKGLEWIGVMATDGSAYYATWTKGRLTISRTSTTVELTITSPTTEDTATYFCARGSGWESYFNTWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 5 aa, synthetic construct
AYDMS

SEQ ID NO: 116 - protein, 16 aa, synthetic construct
IIDTGGSAYYMNWAKG

SEQ ID NO: 117 - protein, 13 aa, synthetic construct
VPWSSDSGSYLNL

SEQ ID NO: 118 - protein, 11 aa, synthetic construct
QASESIGSYLS

SEQ ID NO: 119 - protein, 7 aa, synthetic construct
YASTLAS

SEQ ID NO: 120 - protein, 12 aa, synthetic construct
AGYKNWINDEYP

SEQ ID NO: 121 - protein, 78 aa, synthetic construct
ALVMTQTPSPVSAAVGSTVTIWYQQKPGQPPKLLIYGVPSRFSGSGSGTQFTLTISGVECDDAATYYCFGGGTEVVVK

SEQ ID NO: 122 - protein, 84 aa, synthetic construct
QSVEESGGRLVTPGTPLTLTCTASGFSLSWVRQAPGKGLEWIGRFTISRTSTAVDLKMTSLTTEDTATYFCARWGPGTLVTVSS

SEQ ID NO: 123 - protein, 217 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 448 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0098841 A1
(43) Pub. Date: Apr. 9, 2026

(54) METHOD FOR QUANTIFICATION OF POLYSACCHARIDE CONTENT IN CONJUGATE VACCINES

(71) Applicant: MERCK SHARP & DOHME LLC, Rahway, NJ (US)

(72) Inventors: Zhengwu James DENG, Eagleville, PA (US); Mingxiang LIN, Westfield, NJ (US); Ping ZHUANG, Princeton, NJ (US)

(73) Assignee: MERCK SHARP & DOHME LLC, Rahway, NJ (US)

(21) Appl. No.: 19/111,966

(22) PCT Filed: Sep. 14, 2023
(86) PCT No.: PCT/US2023/032674

§ 371 Date: Mar. 14, 2025

Related U.S. Application Data
(60) Provisional application No. 63/407,373, filed on Sep. 16, 2022.

(51) Int. Cl. G01N 30/86; G01N 30/04; G01N 30/74; G01N 30/88; G01N 33/58; G01N 33/68

(52) U.S. Cl. CPC G01N 30/8631; G01N 30/74; G01N 33/582; G01N 33/6854; G01N 2030/042; G01N 2030/8831; G01N 2030/8836

(57) ABSTRACT

The present disclosure provides novel methods for serotype-specific analysis of compositions comprising one or more polysaccharides. The polysaccharide content can exist as free polysaccharides, or polysaccharide in other forms, such as polysaccharides attached to other molecules or biologics.

### FIELD OF THE INVENTION

The disclosure provides novel methods for serotype-specific analysis of vaccine compositions comprising one or more polysaccharides. The polysaccharide content can exist as free polysaccharides, or polysaccharide in other forms, such as polysaccharides attached to other molecules or biologics.

### BACKGROUND OF THE INVENTION

Streptococcus pneumoniae (S. pneumoniae) is a pathogen that was first isolated by Louis Pasteur and George Sternbern, independently, in 1880. Years later, this was recognized as the main agent causing pneumonia, as well as being a cause of meningitis, otitis media, and other infectious diseases. In many underdeveloped countries, pneumonia caused by S. pneumoniae is the bacterial disease responsible for the major proportion of deaths in children under 5 years of age and adults over 50.

S. pneumoniae exclusively infects humans, with the route of transmission being via saliva droplets from carriers or patients. It is characterized by the frequency with which it colonizes, and by the time it can remain in the nasopharynx without causing disease. Carriers may harbor different serotypes simultaneously or at different times, either continuously or intermittently. S. pneumoniae are encapsulated, aerotolerant anaerobic, gram-positive bacteria. They are immobile, non-sporulating and capable of employing a wide variety of carbohydrates as carbon sources. Microscopically. S. pneumoniae appear as lanceolate diplococci, frequently grouped into short chains, while macroscopically, they present as bright, a-hemolytic, circular colonies. The capsular polysaccharide (CPS) constitutes the outermost layer of the bacterial cell and is the main virulence factor. There are multiple serotypes that can cause a streptococcus infection, with each bacterial serotype having a specific CPS antigen with its own unique structure. In the case of S. pneumoniae, over 90 serotypes have been identified.

Polysaccharide conjugate vaccines are comprised of one or more distinct capsular polysaccharides covalently linked to a carrier, often an immunogenic protein. Manufacturing processes for multivalent polysaccharide vaccines are complex and expensive. Several different fermentation and purification processes must be developed and operated to produce CPS material for a single vaccine drug product. The evolution of high throughput process development (HTPD) for CPS vaccines has been impeded by the lack of rapid assays for CPS quantitation. The challenge in designing streamlined titer assays lies in the intrinsic complexity of CPS. Owing to this constraint, the historical set of CPS titer assays is comprised of complex procedures specific for a given structural moiety/repeating unit.

A number of multivalent pneumococcal vaccines and multivalent pneumococcal conjugate vaccines (PCVs) have been developed, among them being PNEUMOVAX®23, PREVNAR®7, PREVNAR®13, PREVNAR®20 and VAXNEUVANCE™. PCVs are based on carrier protein conjugated multivalent CPS antigens. In all cases, multivalent immunogenic compositions comprising S. pneumoniae polysaccharide or polysaccharide protein conjugates are incorporated as active ingredients in the vaccine drug product. Therefore, identification and/or quantitation of the polysaccharide content in the vaccine is critical for quality control and process monitoring/optimization.

There are 23 serotypes of pneumococcal polysaccharide in PNEUMOVAX®23 (serotypes 1, 2, 3, 4, 5, 6B, 7F, 8, 9N, 9V, 10A, 11A, 12F, 14, 15B, 17F, 18C, 19F, 19A, 20, 22F, 23F, and 33F); and 15 serotypes in VAXNEUVANCE™ (serotypes 1, 3, 4, 5, 6A, 6B, 7F, 9V, 14, 18C, 19A, 19F, 22F, 23F and 33F), V116, an investigational PCV, contains 21 serotypes (serotypes 3, 6A, 7F, 8, 9N, 10A, 11A, 12F, 15A, de-O-acetylated 15B, 16F, 17F, 19A, 20A, 22F, 23A, 23B, 24F, 31, 33F and 35B). Serotype-specific analysis of these investigational vaccines and products is challenging. Currently, plate-reader based enzyme-linked immunosorbent assay (ELISA) is the gold standard assay for serotype-specific polysaccharide analysis. The sandwich ELISA is one format for this assay: however, this method requires two serotype-specific antibodies for capture and detection, and another enzyme-linked species-specific antibody to generate a chemiluminescence signal. In addition, the assay depends on several sensitive bio-critical reagents, has a long incubation/washing time, and requires a series of sample dilutions.

Liquid chromatography methods, including UPLC and HPLC, have also been used for the analysis of a single polysaccharide type. These methods, however, are non-ideal for the analysis of vaccines having multiple polysaccharide serotypes that contain structural similar monosaccharide building blocks. An additional challenge for polysaccharide quantitation using chromatographic methods is a lack of chromophores and fluorophores on polysaccharides. Accordingly, serotype-specific identification and quantitation of multiple polysaccharides are not feasible using current chromatographic methods.

The increasing requirement for multivalent vaccines containing diverse capsular polysaccharides has created an unmet need for a fast and straightforward assay for polysaccharide titer. The invention addresses that unmet need.

### SUMMARY OF THE INVENTION

Provided herein is a novel method for identification and quantification of a free polysaccharide serotype present in a vaccine drug product comprising at least one polysaccharide conjugate, said method comprising the steps:
a) obtaining a standard sample comprising a polysaccharide serotype corresponding to a serotype of the at least one polysaccharide conjugate present in the vaccine drug product;
b) obtaining a vaccine drug product sample stock solution that was prepared from the vaccine drug product;
c) adding to the vaccine drug product sample stock solution, a serospecific anti-polysaccharide antibody corresponding to the polysaccharide serotype of step (a), creating a mixture, wherein the amount of serospecific anti-polysaccharide antibody added is sufficient to ensure that all antibody binding sites on the polysaccharide serotype in the vaccine drug product stock solution are occupied by the corresponding serospecific anti-polysaccharide antibody, to form an antibody-polysaccharide complex (APC) in the mixture;
d) subjecting the APC in the mixture to a chromatographic separation method to provide a quantitative peak area;
e) using a linear fit equation to calculate the amount of the free polysaccharide of the serotype used in step (a) that is present in the mixture, wherein the linear fit equation takes into account the slope and intercept of a standard curve for the polysaccharide serotype corresponding to the polysaccharide serotype of step (a) along with the quantitative peak area generated in step (d); and
   optionally repeating steps (a) through (e) one or more times to identify and/or quantify other polysaccharide serotypes that are present in the vaccine drug product.

Regarding the method above, the standard curve for the polysaccharide serotype corresponding to the polysaccharide serotype of step (a) was generated using a method comprising the following steps:
(i) taking one or more aliquots of the standard sample prepared in step (a);
(ii) diluting one aliquot to a known concentration using a buffer, or diluting multiple aliquots to different known concentrations using a buffer;
(iii) adding to the aliquot(s) made in step (ii) the serospecific anti-polysaccharide antibody specific against the polysaccharide serotype of step (a), creating a binding reaction mixture, wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotypes corresponding to the polysaccharide serotypes of step (a) in each aliquot is saturated with its corresponding serospecific anti-polysaccharide antibody;
(iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and
(v) generating a standard curve using data obtained from the chromatographic separations.

Further provided is a novel method for identification and quantification of a polysaccharide serotype present in a mixture, wherein said mixture comprises one or more polysaccharide serotypes, said method comprising the steps:
a) preparing a standard sample comprising a polysaccharide serotype present in the mixture;
b) preparing a mixture sample stock solution from the mixture;
c) adding to the mixture sample stock solution prepared in step (b), a serospecific anti-polysaccharide antibody corresponding to the polysaccharide serotype of step (a), wherein the amount of serospecific anti-polysaccharide antibody added is sufficient to ensure that all antibody binding sites on the polysaccharide serotype in the mixture stock solution are occupied by the corresponding serospecific anti-polysaccharide antibody, to form an antibody-polysaccharide complex;
d) generating a standard curve for the polysaccharide serotype of step (a), as follows: (i) taking one or more aliquots of the standard sample prepared in step (a); (ii) diluting one aliquot to a known concentration using a buffer, or diluting multiple aliquots to different known concentrations using a buffer; (iii) adding to the aliquot(s) made in step (ii) the serospecific anti-polysaccharide antibody specific against the polysaccharide serotype of step (a), wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotype of step (a) in each aliquot is saturated with its corresponding serospecific anti-polysaccharide antibody; (iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and (v) generating a standard curve using data obtained from the chromatographic separations;
e) subjecting the antibody-polysaccharide complex made in step (c) to a chromatographic separation method to provide a quantitative peak area;
f) using a linear fit equation to calculate the amount of the free polysaccharide of the serotype used in step (a), that is present in the mixture, wherein the linear fit equation takes into account the slope and intercept of the standard curve generated in step (d) along with the APC HPLC peak area generated in step (e); and
g) optionally repeating steps (a) through (f) one or more times to identify and/or quantify other polysaccharide serotypes that are present in the mixture.

The above methods may be referred to singularly, or collectively referred to herein as “methods,” or “the present methods.”

Accordingly, described herein are methods for serotype-specific analysis of compositions comprising one or more polysaccharides, including but not limited to, conjugate vaccines. The polysaccharide content of the compositions being analyzed can exist as free polysaccharides, or polysaccharide in other forms, such as polysaccharides attached to other molecules or biologics.

The present methods are described in detail in the accompanying detailed description below.

Although any methods and materials similar to those described herein can be used in the practice or testing of the present methods and compositions, illustrative methods and materials are now described. Other embodiments, aspects and features of the present methods and compositions are either further described in or will be apparent from the ensuing description, examples and appended claims.

### DETAILED DESCRIPTION OF THE INVENTION

The disclosure provides novel methods for serotype-specific analysis of compositions comprising one or more polysaccharides. The polysaccharide content can exist as free polysaccharides, or polysaccharide in other forms, such as polysaccharides attached to other molecules or biologics.

### Definitions and Abbreviations

The terms used herein have their ordinary meaning and the meaning of such terms is independent at each occurrence thereof. That notwithstanding, and except where stated otherwise, the following definitions apply throughout the specification and claims. Chemical names, common names, and chemical structures may be used interchangeably to describe the same structure. If a chemical or biological compound is referred to using both a structure and a name, and an ambiguity exists between the structure and the name, the structure predominates. These definitions apply regardless of whether a term is used by itself or in combination with other terms, unless otherwise indicated.

As used herein, and throughout this disclosure, the following terms, unless otherwise indicated, shall be understood to have the following meanings:
The term “APC” or “antibody-polysaccharide complex,” is a complex molecule formed by binding a polysaccharide to the antibody against this polysaccharide. Each antibody used in the present methods was generated to selectively target a polysaccharide serotype as an anti-serotype antibody. With this, the “APC” or “antibody-polysaccharide complex,” is formed when a polysaccharide serotype selectively binds to the anti-serotype antibody against the same serotype.

The term “assay standard curve,” as used herein, refers to a standard curve that is the mathematical relationship between two quantities. It is established between signals (the 1^{st} quantity) of standards and predetermined concentrations (or amount) (the 2^{nd} quantity) of the standards. Using the assay described in the Examples below, the standard curve is generated in a linear fashion with X-axis and Y-axis each representing one of the quantities in the relationship. The y-axis represents signals measured from the serotype-specific antibody polysaccharide complex (peak area). The x-axis represents the polysaccharide concentrations or polysaccharide amounts that bind to the antibody in a serotype specific antibody polysaccharide complex. These concentrations (or amount) of the polysaccharide are predetermined for polysaccharide standards and their antibody binding reactions. Once the relationship (linear in this case) between the complex peak area and polysaccharide content of that serotype is established using the standard curve, the polysaccharide concentration (or amount) of a serotype in a vaccine sample can be obtained by measuring its serotype specific antibody polysaccharide complex peak, then converting the peak area to the polysaccharide content of the measured serotype using mathematical relationships with the standard curve. The relationship is demonstrated in Equations-1 and Equation-1a, shown below, using slope and intercept of the linear standard curve.

$$[ DP Ps in binding reaction ] = \frac{DP sample peak area - STD intercept}{STD slope} \qquad Equation - 1$$

$$Ps Amt per injection ( µg ) = \frac{Sample peak area - STD intercept}{STD slope} \qquad Equation - 2$$

The term “drug product formulation buffer,” as used herein, refers to the solution in which the vaccine drug product resides.

The term “polysaccharide standard sample,” as used herein, refers to a polysaccharide sample of a known serotype (known repeating unit structure) with a known concentration. Upon binding to the antibody that is anti this serotype, it forms a serotype specific antibody polysaccharide complex that is used to identify and quantify this polysaccharide. Antibody polysaccharide complexes generated from several different standard concentrations are used to generate a standard curve for polysaccharide quantitation.

The term “serospecific anti-polysaccharide antibody,” as used herein, refers to the antibody that was generated from human or animal species by the immunogenic reaction elicited by a certain polysaccharide serotype. The antibody clones obtained initially were screened against other polysaccharide serotypes to ensure that only the clone specific to the target polysaccharide serotype is selected and used to produce antibodies used in this study. In one embodiment, the serotype-specific antibody is labeled with a fluorescence (FLR) tag. Fluorescence tagged serotype-specific antibodies useful in the present methods may be commercially available or alternatively, can be prepared using methods well-known to the skilled artisan. Non-limiting examples of such methods are disclosed in Chen, et al., BMC Infectious Diseases. 18, 613 (2018); and Cox et al., J. Immunol. 200 (Supp 1), 180 (2018).

The term “serotype-specific knockout sample,” as used herein, refers to a mixture of multiple polysaccharide serotypes in the absence of one specific interested serotype (knockout type). This sample is used as negative control (no binding) in a binding reaction with the antibody target missing the specific interested serotype. In comparison with the binding reaction of antibody binding to its target serotype (positive control standard) which generate antibody polysaccharide complex signal, there is no antibody polysaccharide complex signal or greatly reduced signal from antibody binding to its corresponding serotype knockout sample (negative control). This indicates the antibody binding to polysaccharide is serotype specific (specificity of the antibody).

The term “V116” means an investigational PCV that contains 21 serotypes (serotypes 3, 6A, 7F, 8, 9N, 10A, 11A, 12F, 15A, de-O-acetylated 15B, 16F, 17F, 19A, 20A, 22F, 23A, 23B, 24F, 31, 33F and 35B). V116 is otherwise referred to as PCV21.

The term “vaccine drug product,” as used herein, refers to a research or commercial vaccine containing polysaccharides. Further, the term “vaccine drug product,” as used herein, refers to a research or commercial vaccine containing polysaccharide conjugates. It may contain polysaccharide conjugated to proteins, lipids and other biological or small molecule carriers. It may also contain unconjugated polysaccharides as ingredients of the vaccine drug product. Exemplary vaccine drug products include, but are not limited to, commercial vaccines, such as the pneumococcal vaccine PNEUMOVAX®23 (Merck Sharp & Dohme LLC, Rahway, NJ, USA). Exemplary vaccine drug products include, but are not limited to, commercial vaccines, such as pneumococcal conjugated vaccines (VAXNEUVANCE™ (Merck Sharp & Dohme LLC, Rahway, NJ, USA), PREVNAR 20® (Pfizer Inc., Philadelphia, PA), PREVNAR 13® (Pfizer Inc., Philadelphia, PA), SYNFLORIX® (GlaxoSmithKline Biologicals SA, Rixensart, Belgium)) and meningococcal conjugate vaccines (MENACTRA® (Sanofi Pasteur, Inc., MENVEO® (GlaxoSmithKline Biologicals SA, Rixensart, Belgium)). Exemplary vaccine drug products include V116.

Whenever a range is recited, numbers within the range, as well as the endpoints are contemplated as embodiments of the disclosure. Thus, for example, a “pH of 5 to 9” includes a pH of 5, 6, 7, 8, and 9, as well as any non-whole numbers in between 5 and 9 such as 5.3, 6.7, 8.4, etc.

The following abbreviations are used below, and have the following meanings:

| | |
|---|---|
| APC | Antibody-polysaccharide complex |
| BisTris | 2-bis(2-hydroxyethyl)amino-2(hydroxymethyl)- |
| | 1,3-propanediol |
| CRM197 | Cross-Reacting Material 197 |
| DP | Drug Product |
| FLR | Fluorescence |
| HPLC | High Performance Liquid Chromatography |
| Inj or inj | Injection |
| mAb | Monoclonal antibody |
| PBS | Phosphate-Buffered Saline |
| PCV | Pneumococcal Vaccine |
| Ps | Polysaccharide |
| ST | Serotype |
| STD | Standard |
| Tris | Tris(hydroxymethyl)aminomethane, |
| Vol | Volume |

Provided herein is a novel method for identification and quantification of a free polysaccharide serotype present in a vaccine drug product comprising at least one polysaccharide conjugate, said method comprising the steps:
a) obtaining a standard sample comprising a polysaccharide serotype corresponding to a serotype of the at least one polysaccharide conjugate present in the vaccine drug product;
b) obtaining a vaccine drug product sample stock solution that was prepared from the vaccine drug product;
c) adding to the vaccine drug product sample stock solution, a serospecific anti-polysaccharide antibody corresponding to the polysaccharide serotype of step (a), creating a mixture, wherein the amount of serospecific anti-polysaccharide antibody added is sufficient to ensure that all antibody binding sites on the polysaccharide serotype in the vaccine drug product stock solution are occupied by the corresponding serospecific anti-polysaccharide antibody, to form an antibody-polysaccharide complex (APC) in the mixture;
d) subjecting the APC in the mixture to a chromatographic separation method to provide a quantitative peak area;
e) using a linear fit equation to calculate the amount of the free polysaccharide of the serotype used in step (a) that is present in the mixture, wherein the linear fit equation takes into account the slope and intercept of a standard curve for the polysaccharide serotype corresponding to the polysaccharide serotype of step (a) along with the quantitative peak area generated in step (d); and
   optionally repeating steps (a) through (e) one or more times to identify and/or quantify other polysaccharide serotypes that are present in the vaccine drug product.

Regarding the method above, the standard curve for the polysaccharide serotype corresponding to the polysaccharide serotype of step (a) was generated using a method comprising the following steps:
(i) taking one or more aliquots of the standard sample prepared in step (a);
(ii) diluting one aliquot to a known concentration using a buffer, or diluting multiple aliquots to different known concentrations using a buffer;
(iii) adding to the aliquot(s) made in step (ii) the serospecific anti-polysaccharide antibody specific against the polysaccharide serotype of step (a), creating a binding reaction mixture, wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotypes corresponding to the polysaccharide serotypes of step (a) in each aliquot is saturated with its corresponding serospecific anti-polysaccharide antibody;
(iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and
(v) generating a standard curve using data obtained from the chromatographic separations.

In one aspect, provided herein is a novel method for identification and/or quantification of a polysaccharide serotype present in a vaccine drug product comprising at least one polysaccharide conjugate, said method comprising the steps:
a) obtaining a standard sample comprising a polysaccharide serotype corresponding to a serotype of the at least one polysaccharide conjugate present in the vaccine drug product;
b) obtaining a vaccine drug product sample stock solution that was prepared from the vaccine drug product;
c) adding to the vaccine drug product sample stock solution, a serospecific anti-polysaccharide antibody corresponding to the polysaccharide serotype of step (a), creating a mixture, wherein the amount of serospecific anti-polysaccharide antibody added is sufficient to ensure that all antibody binding sites on the polysaccharide serotype in the vaccine drug product stock solution are occupied by the corresponding serospecific anti-polysaccharide antibody, to form an antibody-polysaccharide complex (APC) in the mixture;
d) subjecting the APC in the mixture to a chromatographic separation method to provide a quantitative peak area;
e) using a linear fit equation to calculate the amount of the free polysaccharide of the serotype used in step (a) that is present in the mixture, wherein the linear fit equation takes into account the slope and intercept of a standard curve for the polysaccharide serotype corresponding to the polysaccharide serotype of step (a) along with the quantitative peak area generated in step (d); and
   optionally repeating steps (a) through (e) one or more times to identify and/or quantify other polysaccharide serotypes that are present in the vaccine drug product.

Regarding the method above, the standard curve for the polysaccharide serotype corresponding to the polysaccharide serotype of step (a) was generated using a method comprising the following steps:
(i) taking one or more aliquots of the standard sample prepared in step (a);
(ii) diluting one aliquot to a known concentration using a buffer, or diluting multiple aliquots to different known concentrations using a buffer;
(iii) adding to the aliquot(s) made in step (ii) the serospecific anti-polysaccharide antibody specific against the polysaccharide serotype of step (a), creating a binding reaction mixture, wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotypes corresponding to the polysaccharide serotypes of step (a) in each aliquot is saturated with its corresponding serospecific anti-polysaccharide antibody;
(iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and
(v) generating a standard curve using data obtained from the chromatographic separations.

In another aspect, provided is a novel method for identification and quantification of a polysaccharide serotype present in a mixture comprising one or more polysaccharide serotypes, said method comprising the steps:
a) preparing a standard sample comprising a polysaccharide serotype present in the mixture;
b) preparing a mixture sample stock solution from the mixture;
c) adding to the mixture sample stock solution prepared in step (b), a serospecific anti-polysaccharide antibody corresponding to the polysaccharide serotype of step (a), wherein the amount of serospecific anti-polysaccharide antibody added is sufficient to ensure that all antibody binding sites on the polysaccharide serotype in the mixture stock solution are occupied by the corresponding serospecific anti-polysaccharide antibody, to form an antibody-polysaccharide complex;
d) generating a standard curve for the polysaccharide serotype of step (a), as follows: (i) taking one or more aliquots of the standard sample prepared in step (a); (ii) diluting one aliquot to a known concentration using a buffer, or diluting multiple aliquots to different known concentrations using a buffer; (iii) adding to the aliquot(s) made in step (ii) the serospecific anti-polysaccharide antibody specific against the polysaccharide serotype of step (a), wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotype of step (a) in each aliquot is saturated with its corresponding serospecific anti-polysaccharide antibody; (iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and (v) generating a standard curve using data obtained from the chromatographic separations;
e) subjecting the antibody-polysaccharide complex made in step (c) to a chromatographic separation method to provide a quantitative peak area;
f) using a linear fit equation to calculate the amount of the free polysaccharide of the serotype used in step (a), that is present in the mixture, wherein the linear fit equation takes into account the slope and intercept of the standard curve generated in step (d) along with the APC HPLC peak area generated in step (e); and
g) optionally repeating steps (a) through (f) one or more times to identify and/or quantify other polysaccharide serotypes that are present in the mixture.

In another aspect, provided herein is a novel method for serotype-specific identification and/or quantification of a free polysaccharide present in a vaccine drug product, said method comprising the steps:
a) preparing a standard sample comprising a polysaccharide serotype present in the vaccine drug product;
b) preparing a vaccine drug product sample stock solution from the vaccine drug product;
c) adding to the vaccine drug product sample stock solution prepared in step (b), a serospecific anti-polysaccharide antibody corresponding to the polysaccharide serotype of step (a), wherein the amount of serospecific anti-polysaccharide antibody added is sufficient to ensure that all antibody binding sites on the polysaccharide serotype in the vaccine drug product stock solution are occupied by the corresponding serospecific anti-polysaccharide antibody, to form an antibody-polysaccharide complex;
d) generating a standard curve for the polysaccharide serotype corresponding to the polysaccharide serotype of step (a), as follows: (i) taking one or more aliquots of the standard sample prepared in step (a); (ii) diluting one aliquot to a known concentration using a buffer, or diluting multiple aliquots to different known concentrations using a buffer; (iii) adding to the aliquot(s) made in step (ii) the serospecific anti-polysaccharide antibody specific against the polysaccharide serotype of step (a), wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotype corresponding the polysaccharide serotype of step (a) in each aliquot is saturated with its corresponding serospecific anti-polysaccharide antibody; (iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and (v) generating a standard curve using data obtained from the chromatographic separations;
e) subjecting the antibody-polysaccharide complex made in step (c) to a chromatographic separation method to provide a quantitative peak area;
f) using a linear fit equation to calculate the amount of the free polysaccharide of the serotype used in step (a), that is present in the mixture, wherein the linear fit equation takes into account the slope and intercept of the standard curve generated in step (d) along with the APC HPLC peak area generated in step (e); and
g) optionally repeating steps (a) through (f) one or more times to identify and/or quantify other polysaccharide serotypes that are present in the vaccine drug product.

Upon binding of a serotype specific anti-polysaccharide antibody to its corresponding polysaccharide serotype, an antibody-polysaccharide complex will be formed. This antibody-polysaccharide complex can be used as surrogate for polysaccharide analysis upon separation of the antibody-polysaccharide complex from unbound antibody.

In one embodiment, the polysaccharide content of interest includes numerous different polysaccharides in free unconjugated form as the active ingredients of a drug or vaccine drug product.

In another embodiment, the polysaccharide content of interest exist as multi-valent mixture of polysaccharides in various conjugated forms as the active ingredients of a drug or vaccine drug product. In a specific embodiment, the polysaccharide content of a vaccine drug product is being analyzed. In another specific embodiment, the vaccine drug product is a conjugate vaccine drug product. In a further embodiment, the vaccine drug product is a non-conjugated vaccine drug product.

In another embodiment, the polysaccharide serotype being quantified is an impurity present in a vaccine drug product of interest.

In still another embodiment, the polysaccharide serotype being quantified exists as unconjugated or conjugated polysaccharide in a food product or diagnostic kits or reagents.

In another embodiment, provided is a method for the quantification and/or identification of free polysaccharide content in a pneumococcal vaccine drug product.

In one embodiment, the pneumococcal vaccine drug product comprises S. pneumoniae polysaccharide or polysaccharide protein conjugates from the following serotypes: 1, 2, 3, 4, 5, 6B, 7F, 8, 9N, 9V, 10A, 11A, 12F, 14, 15B, 17F, 18C, 19F, 19A, 20, 22F, 23F, and 33F.

In another embodiment, the pneumococcal vaccine drug product comprises S. pneumoniae polysaccharide or polysaccharide protein conjugates from the following serotypes: 1, 3, 4, 5, 6A, 6B, 7F, 9V, 14, 18C, 19A, 19F, 22F, 23F and 33F.

In another embodiment, the pneumococcal vaccine drug product comprises S. pneumoniae polysaccharide or polysaccharide protein conjugates from serotypes: 3, 6A, 7F, 8, 9N, 10A, 11A, 12F, 15A, de-O-acetylated 15B, 16F, 17F, 19A, 20A, 22F, 23A, 23B, 24F, 31, 33F and 35B.

In one embodiment, the pneumococcal vaccine drug product is PNEUMOVAX®23, or PREVNAR®7, or PREVNAR®13 or PREVNAR®20, or VAXNEUVANCE™, or V116.

In another embodiment, the protein in a “polysaccharide protein conjugate” is a carrier protein.

In another embodiment, the carrier protein is CRM197.

In the methods of the invention, the detection signal from each polysaccharide antibody complex has a linear response to the antibody corresponded serotype polysaccharide concentration in the vaccine drug product. The method is a serotype specific, precise, robust, accurate method for the identification and quantification of polysaccharides in all types of biological samples derived from both upstream and downstream development and manufacturing processes. Samples with different matrices can be analyzed. Both fluorescence and UV channels that are used for protein or antibody detection can be employed in the use of the methods of the invention.

Non-limiting examples of vaccine drug products having polysaccharide components that can be quantified using the present methods include multivalent immunogenic compositions of pneumococcal polysaccharide or/and polysaccharide-protein conjugates. Non-limiting examples of vaccine drug products having polysaccharide components that can be quantified using the present methods include multivalent immunogenic compositions of S. pneumoniae polysaccharide or/and S. pneumoniae polysaccharide-protein conjugates

Antibodies employed in the present methods may be generated and selected using each individual polysaccharide antigen. The specificity of each antibody to its antigen against other serotypes was confirmed prior to use.

In one embodiment, the vaccine drug product of interest is diluted to a concentration that is suitable for antibody binding.

In one embodiment, the vaccine drug product is binding to an antibody directly in the vaccine drug product formulation buffer for analysis.

In another embodiment, the vaccine drug product is diluted or exchanged in an antibody binding buffer then binds to antibody for analysis.

In embodiments of the invention, a “sufficient” amount of serospecific anti-polysaccharide antibody is added so that the antibody binding sites on the polysaccharide serotype in the vaccine drug product stock solution are occupied by the corresponding serospecific anti-polysaccharide antibody, to form an antibody-polysaccharide complex (APC); in certain embodiments, “sufficient” binding is confirmed through the presence of an un-bound (excess) antibody peak on a chromatogram. In an embodiment, “sufficient” binding means that all of the antibody binding sites are occupied. In another embodiment, “sufficient” binding means that 95% of the binding sites are occupied. In another embodiment, “sufficient” binding means that 96% of the binding sites are occupied. In another embodiment, “sufficient” binding means that 97% of the binding sites are occupied. In another embodiment, “sufficient” binding means that 98% of the binding sites are occupied. In another embodiment, “sufficient” binding means that 99% of the binding sites are occupied.

In one embodiment, the vaccine drug product is pre-purified using one or more purification steps before binding to antibody for analysis. These purification steps may comprise one or more purification techniques, including, but not limited to centrifugation, filtration, affinity capture, chromatographic separation, immunoprecipitation, or a capture step using reactive resins that can conjugate to functional groups present on one or more proteins in the vaccine drug product. Illustrative examples of such resins, include but are not limited to NHS-Activated agarose and maleimide activated resins.

In another embodiment, the vaccine drug product is pre-purified using centrifugation.

In another embodiment, the vaccine drug product is pre-purified using an immunoprecipitation procedure to separate conjugated polysaccharides from unconjugated polysaccharides.

In a further embodiment, the vaccine drug product is pre-purified using affinity capture.

In a specific embodiment, the vaccine drug product is purified using both centrifugation and immunoprecipitation procedures.

In another specific embodiment, the vaccine drug product is purified using centrifugation followed by affinity capture.

In other embodiments, an affinity capture purification step is replaced with a capture step using reactive resins that can conjugate to lysine or thiol groups of CRM197.

Antibody used in this study can be extended to modified antibodies, antibody fragments (FAB or scFV) or synthetic peptides or ligands that are designed to bind polysaccharide serotypes with specificity.

Fluorescence-labeled antibodies have been employed in this study. These labeled antibodies maintain the specificity against their target polysaccharide serotypes. The fluorescent label grants unique spectroscopic properties to the tagged antibody, which allows the antibody or antibody bound species, such as APC, to be detected at a unique wavelength on the instrument. Antibodies labeled by other tags, that can generate unique signals, such as radioactive elements or isotopes, can also be applied in this assay.

In one embodiment, the serotype-specific antibody is a fluorescence-labeled antibody.

The vaccine drug product of interest is bound to antibody in an optimal binding buffer, temperature and incubated for a period of time before analysis. Generally, an optimal binding buffer has pH from 5-9, salt concentration from 0-0.7 M. Binding temperature can be from 20-50° C. The incubation time for a binding reaction can be from 0.5 to 24 hours. In one embodiment, the buffer comprises a salt. In a specific embodiment, the buffer comprises a chloride salt.

In certain embodiments, the antibody binding reaction is performed in a phosphate buffer with a salt concentration of 0.05 to 0.5 M and a pH range from 6 to 8. The reaction can be incubated at ambient temperature from one to five hours.

In certain embodiments, the antibody binding reaction is performed in an organic salt buffer, such as Tris(tris(hydroxymethyl)aminomethane) with a salt concentration of 0.05 to 0.5 M and a pH range from 6 to 8. In certain embodiments, the antibody binding reaction is performed in an organic salt buffer, such as Tris(tris(hydroxymethyl)aminomethane) with a salt concentration of 0.05 to 1 M and a pH range from 6 to 8. The reaction can be incubated at ambient temperature from one to five hours.

In certain embodiments, the antibody binding reaction is performed in a buffer mentioned above with a certain percentage of protein solubilization detergents, such as polysorbates (i.e., Ps-20 or Ps-80, etc.).

In certain embodiments, the antibody binding reaction is performed in a mixture of two or more buffers with an appropriate amount of protein solubilization detergents.

In one embodiment, the present methods are based on analysis performed on separation of antibody complex, particularly with various chromatography separation techniques.

In certain embodiments, the sample is separated by size-exclusion chromatography (SEC) performed on a high-performance liquid chromatography (HPLC), an ultra-performance liquid chromatography (UPLC), or a fast protein liquid chromatography (FPLC) system.

In certain embodiments, the sample is separated by ion-exchange chromatography (IEX) performed on a high-performance liquid chromatography (HPLC), an ultra-performance liquid chromatography (UPLC), or a fast protein liquid chromatography (FPLC) system.

In certain embodiments, the sample is separated by chromatography separation based on the sample's hydrophobic or hydrophilic properties performed on a high-performance liquid chromatography (HPLC), an ultra-performance liquid chromatography (UPLC), or a fast protein liquid chromatography (FPLC) system.

In certain embodiments, the samples are separated by capillary electrophoresis separation such as capillary zone electrophoresis (CZE), or capillary gel electrophoresis (CE).

In certain embodiments, the separations are performed using size-exclusion columns with appropriate pore size and particle size in a buffered mobile phase.

In certain embodiments, the separations are performed using size-exclusion columns selected from Tosoh TSKgel columns, such as the TSKgel SW or Tosoh TSKgel PW columns in a buffered mobile phase.

In certain embodiments, the separations are performed using size-exclusion columns selected from Shodex columns, such as Shodex KW, LW, SB or LB columns in a buffered mobile phase.

The mobile phases used for the separation are aqueous buffer solutions or aqueous buffer solutions containing up to about 10% organic solvent, such as acetonitrile or methanol.

In certain embodiments, the mobile phase used for the separation is a buffer made from inorganic salt such as phosphate buffer with a pH from 6-9.

In certain embodiments, the mobile phase used for the separation is a buffer comprising an organic salt, such as Tris, Bis-Tris, Bis-Tris Propane. HEPES, MOPS with a pH from 6-9. In one embodiment, the salt is an inorganic salt, such as NaCl or KCl. In another embodiment, the salt is an organic salt.

In certain embodiments, the mobile phase used for the separation is a histidine buffer or buffer made from other amino acids with a pH from 6-9 with an appropriate salt concentration. Separations using HPLC can be run using isocratic mobile phase at a flow rate from 0.4 mL/min to 1.0 mL/min. A typical mobile phase is a neutral or near neutral pH salt buffer with varied salt concentrations. In one embodiment, the HPLC mobile phase is 10 mM Bis-Tris, 150 mM NaCl, pH 6.5-7.5 buffer. In another embodiment, the HPLC mobile phase is 10 mM Bis-Tris, 300 mM NaCl, pH 6.5-7.5 buffer. In another embodiment, the HPLC mobile phase is 10 mM Bis-Tris, 500 mM NaCl. pH 6.5-7.5 buffer. In some cases, HPLC mobile phase is PBS buffer.

SEC columns useful in the present methods can be obtained commercially. In one embodiment, the HPLC column is a Tosoh TSKgel-GMPWxL column (Tosoh Bioscience, Japan). In another embodiment, the column is a Shodex protein KW-803 column (Showa Denko America. Inc., NY). In another embodiment, the column is a Sepax SRT SEC-1000 column (Sepax Technologies. Newark, DE). In one embodiment, the HPLC column is a Tosoh TSKgel-G4000PWxL column (Tosoh Bioscience, Japan). In one embodiment, the HPLC column is a Tosoh TSKgel-G5000PWxL column (Tosoh Bioscience, Japan). The column temperature is set at a certain temperature from 20° C. to 40° C. A typical column temperature is 30° C. or 35° C. The HPLC autosampler is set at a temperature from 4° C. to 10° C. A typical HPLC autosampler temperature is set at 6° C. or 8° C. The HPLC run time is from 10 to 30 minutes. A typical HPLC run time is 20 or 25 minutes.

Once antibody binds to a polysaccharide, it forms a polysaccharide antibody complex having different size or physico-chemical properties that can be identified on a chromatogram after separation and detection. In one embodiment, the samples are detected and quantified using an Ultraviolet (UV) detector. In another embodiments, the samples are detected and quantified using a Fluorescence (FLR) detector. In another embodiment, the samples are detected and quantified using refractive index (RI), Charged Aerosol Detection (CAD), light scattering (LS) detector, mass spectrometry (MS), or pulsed amperometric detection (PAD).

In certain embodiments of the present methods, the concentration of polysaccharide of interest in the vaccine drug product is determined by comparing the polysaccharide antibody peak area with a linear standard curve. The polysaccharide antibody peak area of the sample is within the range of the standard curve. The standard curve is generated by binding a mono-valent polysaccharide standard to its serotype specific antibody at several polysaccharide concentrations. The intercept (STD Intercept) and slope (STD Slope) of the standard curve can be calculated out by software. The test sample polysaccharide concentration can then be calculated using the methods described below in Example 6.

In certain embodiments, such quantitative analysis of the vaccine drug product is performed by directly comparing the peak area from sample polysaccharide antibody complex to peak area of a reference sample.

In one embodiment, a multiplex assay is used to detect and analyze APCs that are made using the present methods, and that comprise fluorescence-labeled serotype-specific antibodies. A multiplex assay can be used to simultaneously detect distinctive FLR tags at multiple wavelengths. See Francisco-Cruz, et. al. (2020) Multiplex Immunofluorescence Assays. In: Thurin, M., Cesano, A., Marincola, F (eds) Biomarkers for Immunotherapy of Cancer. Methods in Molecular Biology, vol 2055. Humana, New York, NY.

In one embodiment, the present methods are used to identify and/or quantify all polysaccharide serotypes present in a mixture, wherein the mixture contains 7, 8, 9, 10, 11, 12, 13, 14, 15, 16, 17, 18, 19, 20, 21, 22, 23, 24, 25, 26, 27, 28, 29 or 30 different polysaccharide serotypes. In another embodiment the polysaccharide serotypes are S. pneumoniae polysaccharide serotypes. In another embodiment, the mixture comprises S. pneumoniae polysaccharide serotype 3.

In another embodiment, the present methods are used to identify and/or quantify all polysaccharide serotypes present in a vaccine drug product, wherein the vaccine drug product contains 7, 8, 9, 10, 11, 12, 13, 14, 15, 16, 17, 18, 19, 20, 21, 22, 23, 24, 25, 26, 27, 28, 29 or 30 different polysaccharide serotypes. In another embodiment, the polysaccharide serotypes are S. pneumoniae polysaccharide serotypes. In another embodiment, the vaccine drug product comprises S. pneumoniae polysaccharide serotype 3.

In another embodiment, the present methods are used to identify and/or quantify all polysaccharide serotypes present in a second vaccine drug product.

In one embodiment, provided are the present methods, wherein an anti-serotype antibody binds a S. pneumoniae ST-3 polysaccharide to form an antibody-polysaccharide complex, wherein the anti-serotype antibody comprises the following six CDRs:
i. a light chain CDR 1 comprising an amino acid sequence of SEQ ID NO: 1, or a functional variant thereof;
ii. a light chain CDR 2 comprising an amino acid sequence of SEQ ID NO: 2, or a functional variant thereof;
iii. a light chain CDR 3 comprising an amino acid sequence of SEQ ID NO: 3, or a functional variant thereof;
iv. a heavy chain CDR 4 comprising an amino acid sequence of SEQ ID NO: 4, or a functional variant thereof;
v. a heavy chain CDR 5 comprising an amino acid sequence of SEQ ID NO: 5, or a functional variant thereof;
vi. a heavy chain CDR 6 comprising an amino acid sequence of SEQ ID NO: 6, or a functional variant thereof.

In another embodiment, provided are the present methods, wherein the anti-serotype antibody binds a S. pneumoniae ST-3 polysaccharide to form an antibody-polysaccharide complex, wherein the anti-serotype antibody comprises:
i. a variable light chain comprising an amino acid sequence of SEQ ID NO: 7, or a functional variant thereof; and
ii. a variable heavy chain comprising an amino acid sequence of SEQ ID NO: 8, or a functional variant thereof; and

In another embodiment, provided are the present methods, wherein the anti-serotype antibody binds a S. pneumoniae ST-3 polysaccharide to form an antibody-polysaccharide complex, wherein the anti-serotype antibody comprises:
i. a full light chain comprising an amino acid sequence of SEQ ID NO: 9, or a functional variant thereof; and
ii. a full heavy chain comprising an amino acid sequence of SEQ ID NO: 10, or a functional variant thereof.

In one embodiment, provided are the present methods, wherein an anti-serotype antibody binds a S. pneumoniae ST-4 polysaccharide to form an antibody-polysaccharide complex, wherein the anti-serotype antibody comprises the following six CDRs:
i. a light chain CDR 1 comprising an amino acid sequence of SEQ ID NO: 11, or a functional variant thereof;
ii. a light chain CDR 2 comprising an amino acid sequence of SEQ ID NO: 12, or a functional variant thereof;
iii. a light chain CDR 3 comprising an amino acid sequence of SEQ ID NO: 13, or a functional variant thereof;
iv. a heavy chain CDR 4 comprising an amino acid sequence of SEQ ID NO: 14, or a functional variant thereof;
v. a heavy chain CDR 5 comprising an amino acid sequence of SEQ ID NO: 15, or a functional variant thereof;
vi. a heavy chain CDR 6 comprising an amino acid sequence of SEQ ID NO: 16, or a functional variant thereof.

In another embodiment, provided are the present methods, wherein the anti-serotype antibody binds a S. pneumoniae ST-4 polysaccharide to form an antibody-polysaccharide complex, wherein the anti-serotype antibody comprises:
i. a variable light chain comprising an amino acid sequence of SEQ ID NO: 17, or a functional variant thereof; and
ii. a variable heavy chain comprising an amino acid sequence of SEQ ID NO: 18, or a functional variant thereof; and

In another embodiment, provided are the present methods, wherein the anti-serotype antibody binds a S. pneumoniae ST-4 polysaccharide to form an antibody-polysaccharide complex, wherein the anti-serotype antibody comprises;
i. a full light chain comprising an amino acid sequence of SEQ ID NO: 19, or a functional variant thereof; and
ii. a full heavy chain comprising an amino acid sequence of SEQ ID NO: 20, or a functional variant thereof.

In one embodiment, provided are the present methods, wherein an anti-serotype antibody binds a S. pneumoniae ST-5 polysaccharide to form an antibody-polysaccharide complex, wherein the anti-serotype antibody comprises the following six CDRs:
i. a light chain CDR 1 comprising an amino acid sequence of SEQ ID NO: 21, or a functional variant thereof;
ii. a light chain CDR 2 comprising an amino acid sequence of SEQ ID NO: 22, or a functional variant thereof;
iii. a light chain CDR 3 comprising an amino acid sequence of SEQ ID NO: 23, or a functional variant thereof;
iv. a heavy chain CDR 4 comprising an amino acid sequence of SEQ ID NO: 24, or a functional variant thereof;
v. a heavy chain CDR 5 comprising an amino acid sequence of SEQ ID NO: 25, or a functional variant thereof;
vi. a heavy chain CDR 6 comprising an amino acid sequence of SEQ ID NO: 26, or a functional variant thereof.

In another embodiment, provided are the present methods, wherein the anti-serotype antibody binds a S. pneumoniae ST-5 polysaccharide to form an antibody-polysaccharide complex, wherein the anti-serotype antibody comprises:
i. a variable light chain comprising an amino acid sequence of SEQ ID NO: 27, or a functional variant thereof; and
ii. a variable heavy chain comprising an amino acid sequence of SEQ ID NO: 28, or a functional variant thereof; and

In another embodiment, provided are the present methods, wherein the anti-serotype antibody binds a S. pneumoniae ST-5 polysaccharide to form an antibody-polysaccharide complex, wherein the anti-serotype antibody comprises:
i. a full light chain comprising an amino acid sequence of SEQ ID NO: 29, or a functional variant thereof; and
ii. a full heavy chain comprising an amino acid sequence of SEQ ID NO: 30, or a functional variant thereof.

In one embodiment, provided are the present methods, wherein an anti-serotype antibody binds a S. pneumoniae ST-6A polysaccharide to form an antibody-polysaccharide complex, wherein the anti-serotype antibody comprises the following six CDRs:
i. a light chain CDR 1 comprising an amino acid sequence of SEQ ID NO: 31, or a functional variant thereof;
ii. a light chain CDR 2 comprising an amino acid sequence of SEQ ID NO: 32, or a functional variant thereof;
iii. a light chain CDR 3 comprising an amino acid sequence of SEQ ID NO: 33, or a functional variant thereof;
iv. a heavy chain CDR 4 comprising an amino acid sequence of SEQ ID NO: 34, or a functional variant thereof;
v. a heavy chain CDR 5 comprising an amino acid sequence of SEQ ID NO: 35, or a functional variant thereof;
vi. a heavy chain CDR 6 comprising an amino acid sequence of SEQ ID NO: 36, or a functional variant thereof.

In another embodiment, provided are the present methods, wherein the anti-serotype antibody binds a S. pneumoniae ST-6A polysaccharide to form an antibody-polysaccharide complex, wherein the anti-serotype antibody comprises:
i. a variable light chain comprising an amino acid sequence of SEQ ID NO: 37, or a functional variant thereof; and
ii. a variable heavy chain comprising an amino acid sequence of SEQ ID NO: 38, or a functional variant thereof; and

In another embodiment, provided are the present methods, wherein the anti-serotype antibody binds a S. pneumoniae ST-6A polysaccharide to form an antibody-polysaccharide complex, wherein the anti-serotype antibody comprises:
i. a full light chain comprising an amino acid sequence of SEQ ID NO: 39, or a functional variant thereof; and
ii. a full heavy chain comprising an amino acid sequence of SEQ ID NO: 40, or a functional variant thereof.

In one embodiment, provided are the present methods, wherein an anti-serotype antibody binds a S. pneumoniae ST-6B polysaccharide to form an antibody-polysaccharide complex, wherein the anti-serotype antibody comprises the following six CDRs:
i. a light chain CDR 1 comprising an amino acid sequence of SEQ ID NO: 41, or a functional variant thereof;
ii. a light chain CDR 2 comprising an amino acid sequence of SEQ ID NO: 42, or a functional variant thereof;
iii. a light chain CDR 3 comprising an amino acid sequence of SEQ ID NO: 43, or a functional variant thereof;
iv. a heavy chain CDR 4 comprising an amino acid sequence of SEQ ID NO: 44, or a functional variant thereof;
v. a heavy chain CDR 5 comprising an amino acid sequence of SEQ ID NO: 45, or a functional variant thereof;
vi. a heavy chain CDR 6 comprising an amino acid sequence of SEQ ID NO: 46, or a functional variant thereof.

In another embodiment, provided are the present methods, wherein the anti-serotype antibody binds a S. pneumoniae ST-6B polysaccharide to form an antibody-polysaccharide complex, wherein the anti-serotype antibody comprises:
i. a variable light chain comprising an amino acid sequence of SEQ ID NO: 47, or a functional variant thereof; and
ii. a variable heavy chain comprising an amino acid sequence of SEQ ID NO: 48, or a functional variant thereof; and

In another embodiment, provided are the present methods, wherein the anti-serotype antibody binds a S. pneumoniae ST-6B polysaccharide to form an antibody-polysaccharide complex, wherein the anti-serotype antibody comprises:
i. a full light chain comprising an amino acid sequence of SEQ ID NO: 49, or a functional variant thereof; and
ii. a full heavy chain comprising an amino acid sequence of SEQ ID NO: 50, or a functional variant thereof.

In one embodiment, provided are the present methods, wherein an anti-serotype antibody binds a S. pneumoniae ST-7F polysaccharide to form an antibody-polysaccharide complex, wherein the anti-serotype antibody comprises the following six CDRs:
i. a light chain CDR 1 comprising an amino acid sequence of SEQ ID NO: 51, or a functional variant thereof;
ii. a light chain CDR 2 comprising an amino acid sequence of SEQ ID NO: 52, or a functional variant thereof;
iii. a light chain CDR 3 comprising an amino acid sequence of SEQ ID NO: 53, or a functional variant thereof;
iv. a heavy chain CDR 4 comprising an amino acid sequence of SEQ ID NO: 54, or a functional variant thereof;
v. a heavy chain CDR 5 comprising an amino acid sequence of SEQ ID NO: 55, or a functional variant thereof;
vi. a heavy chain CDR 6 comprising an amino acid sequence of SEQ ID NO: 56, or a functional variant thereof.

In another embodiment, provided are the present methods, wherein the anti-serotype antibody binds a S. pneumoniae ST-7F polysaccharide to form an antibody-polysaccharide complex, wherein the anti-serotype antibody comprises:
i. a variable light chain comprising an amino acid sequence of SEQ ID NO: 57, or a functional variant thereof; and
ii. a variable heavy chain comprising an amino acid sequence of SEQ ID NO: 58, or a functional variant thereof; and

In another embodiment, provided are the present methods, wherein the anti-serotype antibody binds a S. pneumoniae ST-7F polysaccharide to form an antibody-polysaccharide complex, wherein the anti-serotype antibody comprises:
i. a full light chain comprising an amino acid sequence of SEQ ID NO: 59, or a functional variant thereof; and
ii. a full heavy chain comprising an amino acid sequence of SEQ ID NO: 60, or a functional variant thereof.

In one embodiment, provided are the present methods, wherein an anti-serotype antibody binds a S. pneumoniae ST-9V polysaccharide to form an antibody-polysaccharide complex, wherein the anti-serotype antibody comprises the following six CDRs:
i. a light chain CDR 1 comprising an amino acid sequence of SEQ ID NO: 61, or a functional variant thereof;
ii. a light chain CDR 2 comprising an amino acid sequence of SEQ ID NO: 62, or a functional variant thereof;
iii. a light chain CDR 3 comprising an amino acid sequence of SEQ ID NO: 63, or a functional variant thereof;
iv. a heavy chain CDR 4 comprising an amino acid sequence of SEQ ID NO: 64, or a functional variant thereof;
v. a heavy chain CDR 5 comprising an amino acid sequence of SEQ ID NO: 65, or a functional variant thereof;
vi. a heavy chain CDR 6 comprising an amino acid sequence of SEQ ID NO: 66, or a functional variant thereof.

In another embodiment, provided are the present methods, wherein the anti-serotype antibody binds a S. pneumoniae ST-9V polysaccharide to form an antibody-polysaccharide complex, wherein the anti-serotype antibody comprises:
i. a variable light chain comprising an amino acid sequence of SEQ ID NO: 67, or a functional variant thereof; and
ii. a variable heavy chain comprising an amino acid sequence of SEQ ID NO: 68, or a functional variant thereof; and

In another embodiment, provided are the present methods, wherein the anti-serotype antibody binds a S. pneumoniae ST-9V polysaccharide to form an antibody-polysaccharide complex, wherein the anti-serotype antibody comprises:
i. a full light chain comprising an amino acid sequence of SEQ ID NO: 69, or a functional variant thereof; and
ii. a full heavy chain comprising an amino acid sequence of SEQ ID NO: 70, or a functional variant thereof.

In one embodiment, provided are the present methods, wherein an anti-serotype antibody binds a S. pneumoniae ST-11A polysaccharide to form an antibody-polysaccharide complex, wherein the anti-serotype antibody comprises the following six CDRs:
i. a light chain CDR 1 comprising an amino acid sequence of SEQ ID NO: 71, or a functional variant thereof;
ii. a light chain CDR 2 comprising an amino acid sequence of SEQ ID NO: 72, or a functional variant thereof;
iii. a light chain CDR 3 comprising an amino acid sequence of SEQ ID NO: 73, or a functional variant thereof;
iv. a heavy chain CDR 4 comprising an amino acid sequence of SEQ ID NO: 74, or a functional variant thereof;
v. a heavy chain CDR 5 comprising an amino acid sequence of SEQ ID NO: 75, or a functional variant thereof;
vi. a heavy chain CDR 6 comprising an amino acid sequence of SEQ ID NO: 76, or a functional variant thereof.

In another embodiment, provided are the present methods, wherein the anti-serotype antibody binds a S. pneumoniae ST-11A polysaccharide to form an antibody-polysaccharide complex, wherein the anti-serotype antibody comprises:
i. a variable light chain comprising an amino acid sequence of SEQ ID NO: 77, or a functional variant thereof; and
ii. a variable heavy chain comprising an amino acid sequence of SEQ ID NO: 78, or a functional variant thereof; and

In another embodiment, provided are the present methods, wherein the anti-serotype antibody binds a S. pneumoniae ST-11A polysaccharide to form an antibody-polysaccharide complex, wherein the anti-serotype antibody comprises:
i. a full light chain comprising an amino acid sequence of SEQ ID NO: 79, or a functional variant thereof; and
ii. a full heavy chain comprising an amino acid sequence of SEQ ID NO: 80, or a functional variant thereof.

In one embodiment, provided are the present methods, wherein the anti-serotype antibody binds a S. pneumoniae ST-14 polysaccharide to form an antibody-polysaccharide complex, wherein the anti-serotype antibody comprises:
i. a variable light chain comprising an amino acid sequence of SEQ ID NO: 81, or a functional variant thereof; and
ii. a variable heavy chain comprising an amino acid sequence of SEQ ID NO: 82, or a functional variant thereof; and

In another embodiment, provided are the present methods, wherein the anti-serotype antibody binds a S. pneumoniae ST-14 polysaccharide to form an antibody-polysaccharide complex, wherein the anti-serotype antibody comprises:
i. a full light chain comprising an amino acid sequence of SEQ ID NO: 83, or a functional variant thereof; and
ii. a full heavy chain comprising an amino acid sequence of SEQ ID NO: 84, or a functional variant thereof.

In one embodiment, provided are the present methods, wherein an anti-serotype antibody binds a S. pneumoniae ST-19A polysaccharide to form an antibody-polysaccharide complex, wherein the anti-serotype antibody comprises the following six CDRs:
i. a light chain CDR 1 comprising an amino acid sequence of SEQ ID NO: 85, or a functional variant thereof;
ii. a light chain CDR 2 comprising an amino acid sequence of SEQ ID NO: 86, or a functional variant thereof;
iii. a light chain CDR 3 comprising an amino acid sequence of SEQ ID NO: 87, or a functional variant thereof;
iv. a heavy chain CDR 4 comprising an amino acid sequence of SEQ ID NO: 88, or a functional variant thereof;
v. a heavy chain CDR 5 comprising an amino acid sequence of SEQ ID NO: 89, or a functional variant thereof;
vi. a heavy chain CDR 6 comprising an amino acid sequence of SEQ ID NO: 90, or a functional variant thereof.

In another embodiment, provided are the present methods, wherein the anti-serotype antibody binds a S. pneumoniae ST-19A polysaccharide to form an antibody-polysaccharide complex, wherein the anti-serotype antibody comprises:
i. a variable light chain comprising an amino acid sequence of SEQ ID NO: 91, or a functional variant thereof; and
ii. a variable heavy chain comprising an amino acid sequence of SEQ ID NO: 92, or a functional variant thereof; and

In another embodiment, provided are the present methods, wherein the anti-serotype antibody binds a S. pneumoniae ST-19A polysaccharide to form an antibody-polysaccharide complex, wherein the anti-serotype antibody comprises:
i. a full light chain comprising an amino acid sequence of SEQ ID NO: 93, or a functional variant thereof; and
ii. a full heavy chain comprising an amino acid sequence of SEQ ID NO: 94, or a functional variant thereof.

In one embodiment, provided are the present methods, wherein an anti-serotype antibody binds a S. pneumoniae ST-22F polysaccharide to form an antibody-polysaccharide complex, wherein the anti-serotype antibody comprises the following six CDRs:
i. a light chain CDR 1 comprising an amino acid sequence of SEQ ID NO: 95, or a functional variant thereof;
ii. a light chain CDR 2 comprising an amino acid sequence of SEQ ID NO: 96, or a functional variant thereof;
iii. a light chain CDR 3 comprising an amino acid sequence of SEQ ID NO: 97, or a functional
iv. a heavy chain CDR 4 comprising an amino acid sequence of SEQ ID NO: 98, or a functional variant thereof;
v. a heavy chain CDR 5 comprising an amino acid sequence of SEQ ID NO: 99, or a functional variant thereof;
vi. a heavy chain CDR 6 comprising an amino acid sequence of SEQ ID NO: 100, or a functional variant thereof.

In another embodiment, provided are the present methods, wherein the anti-serotype antibody binds a S. pneumoniae ST-22F polysaccharide to form an antibody-polysaccharide complex, wherein the anti-serotype antibody comprises:
i. a variable light chain comprising an amino acid sequence of SEQ ID NO: 101, or a functional variant thereof; and
ii. a variable heavy chain comprising an amino acid sequence of SEQ ID NO: 102, or a functional variant thereof; and

In another embodiment, provided are the present methods, wherein the anti-serotype antibody binds a S. pneumoniae ST-22F polysaccharide to form an antibody-polysaccharide complex, wherein the anti-serotype antibody comprises:
i. a full light chain comprising an amino acid sequence of SEQ ID NO: 103, or a functional variant thereof; and
ii. a full heavy chain comprising an amino acid sequence of SEQ ID NO: 104, or a functional variant thereof.

In one embodiment, provided are the present methods, wherein an anti-serotype antibody binds a S. pneumoniae ST-23F polysaccharide to form an antibody-polysaccharide complex, wherein the anti-serotype antibody comprises the following six CDRs:
i. a light chain CDR 1 comprising an amino acid sequence of SEQ ID NO: 105, or a functional variant thereof;
ii. a light chain CDR 2 comprising an amino acid sequence of SEQ ID NO: 106, or a functional variant thereof;
iii. a light chain CDR 3 comprising an amino acid sequence of SEQ ID NO: 107, or a functional variant thereof;
iv. a heavy chain CDR 4 comprising an amino acid sequence of SEQ ID NO: 108, or a functional variant thereof;
v. a heavy chain CDR 5 comprising an amino acid sequence of SEQ ID NO: 109, or a functional variant thereof;
vi. a heavy chain CDR 6 comprising an amino acid sequence of SEQ ID NO: 110, or a functional variant thereof.

In another embodiment, provided are the present methods, wherein the anti-serotype antibody binds a S. pneumoniae ST-23F polysaccharide to form an antibody-polysaccharide complex, wherein the anti-serotype antibody comprises:
i. a variable light chain comprising an amino acid sequence of SEQ ID NO: 111, or a functional variant thereof; and
ii. a variable heavy chain comprising an amino acid sequence of SEQ ID NO: 112, or a functional variant thereof; and

In another embodiment, provided are the present methods, wherein the anti-serotype antibody binds a S. pneumoniae ST-23F polysaccharide to form an antibody-polysaccharide complex, wherein the anti-serotype antibody comprises:
i. a full light chain comprising an amino acid sequence of SEQ ID NO: 113, or a functional variant thereof; and
ii. a full heavy chain comprising an amino acid sequence of SEQ ID NO: 114, or a functional variant thereof.

In one embodiment, provided are the present methods, wherein an anti-serotype antibody binds a S. pneumoniae ST-33F polysaccharide to form an antibody-polysaccharide complex, wherein the anti-serotype antibody comprises the following six CDRs:
i. a light chain CDR 1 comprising an amino acid sequence of SEQ ID NO: 115, or a functional variant thereof;
ii. a light chain CDR 2 comprising an amino acid sequence of SEQ ID NO: 116, or a functional variant thereof;
iii. a light chain CDR 3 comprising an amino acid sequence of SEQ ID NO: 117, or a functional variant thereof;
iv. a heavy chain CDR 4 comprising an amino acid sequence of SEQ ID NO: 118, or a functional variant thereof;
v. a heavy chain CDR 5 comprising an amino acid sequence of SEQ ID NO: 119, or a functional variant thereof;
vi. a heavy chain CDR 6 comprising an amino acid sequence of SEQ ID NO: 120, or a functional variant thereof.

In another embodiment, provided are the present methods, wherein the anti-serotype antibody binds a S. pneumoniae ST-33F polysaccharide to form an antibody-polysaccharide complex, wherein the anti-serotype antibody comprises:
i. a variable light chain comprising an amino acid sequence of SEQ ID NO: 121, or a functional variant thereof; and
ii. a variable heavy chain comprising an amino acid sequence of SEQ ID NO: 122, or a functional variant thereof; and

In another embodiment, provided are the present methods, wherein the anti-serotype antibody binds a S. pneumoniae ST-33F polysaccharide to form an antibody-polysaccharide complex, wherein the anti-serotype antibody comprises:
i. a full light chain comprising an amino acid sequence of SEQ ID NO: 123, or a functional variant thereof; and
ii. a full heavy chain comprising an amino acid sequence of SEQ ID NO: 124, or a functional variant thereof.

In another embodiment the invention provides a monoclonal antibody (mAb), or a functional variant thereof, that specifically binds to a pneumococcal serotype (ST) capsular polysaccharide (PnPs), wherein said mAb comprises:
a) six complementarity determining regions (CDRs) selected from the group consisting of SEQ. ID. NOs.: 1-6, 11-16, 21-26, 31-36, 41-46, 51-56, 61-66, 71-76, 85-90, 95-100, 105-110 and 115-120;
b) a variable heavy chain and a variable light chain having an amino acid sequence selected from the group consisting of SEQ ID NOs: 7-8, 17-18, 27-28, 37-38, 47-48, 57-58, 67-68, 77-78, 81-82, 91-92, 101-102, 111-112 and 121-122; or
c) a full length light chain and a full length heavy chain having an amino acid sequence selected from the group consisting of SEQ ID NOs: 9-10, 19-20, 29-30, 39-40, 49-50, 59-60, 69-70, 79-80, 83-84, 93-94, 103-104, 113-114 and 123-124.

In another embodiment the invention provides the monoclonal antibody above, wherein the mAb comprises six CDRs selected from the group consisting of SEQ. ID. NOs. 1-6, 11-16, 21-26, 31-36, 41-46, 51-56, 61-66, 71-76, 85-90, 95-100, 105-110 and 115-120.

In another embodiment the invention provides the monoclonal antibody above, wherein the mAb comprises a variable heavy chain and a variable light chain having an amino acid sequence selected from the group consisting of SEQ ID NOs: 7-8, 17-18, 27-28, 37-38, 47-48, 57-58, 67-68, 77-78, 81-82, 91-92, 101-102, 111-112 and 121-122.

In another embodiment the invention provides the monoclonal antibody above, wherein the mAb comprises a full length light chain and a full length heavy chain having an amino acid sequence selected from the group consisting of SEQ ID NOs: 9-10, 19-20, 29-30, 39-40, 49-50, 59-60, 69-70, 79-80, 83-84, 93-94, 103-104, 113-114 and 123-124.

In one aspect, some of the antibodies used in the present methods comprise the following complementarity determining regions (CDRs), variable heavy chains, variable light chains, full length heavy chains and/or full-length light chains:

| NAME | A.A. Sequence | SEQ. ID. NO. |
|---|---|---|
| ST-3 Monoclonal | QASQSIGSSLA | SEQ. ID. NO.: 1 |
| Antibody (Human) | | |
| Light Chain CDR 1 | | |
| ST-3 Monoclonal | QASKLAS | SEQ. ID. NO.: 2 |
| Antibody (Human) | | |
| Light Chain CDR 2 | | |
| ST-3 Monoclonal | QCTGNGGDFIGA | SEQ. ID. NO.: 3 |
| Antibody (Human) | | |
| Light Chain CDR 3 | | |
| ST-3 Monoclonal | SYYVR | SEQ. ID. NO.: 4 |
| Antibody (Human) | | |
| Heavy Chain CDR 4 | | |
| ST-3 Monoclonal | IISDSGSTYYASWAKG | SEQ. ID. NO.: 5 |
| Antibody (Human) | | |
| Heavy Chain CDR 5 | | |
| ST-3 Monoclonal | GSGYTIPTDL | SEQ. ID. NO.: 6 |
| Antibody (Human) | | |
| Heavy Chain CDR 6 | | |
| ST-3 Monoclonal | DPVMTQTPASVSEPVGGTVTIKCWYQQKPGQR | SEQ. ID. NO.: 7 |
| Antibody (Human) | PKLLIYGVPSRFKGSRSGTEFTLTISDLECADAA | |
| Variable Light Chain | TYYCFGGGTEVVVK | |
| ST-3 Monoclonal | QSLEESGGGLVTPGTPLTLTCTASGFSLSWVRQ | SEQ. ID. NO.: 8 |
| Antibody (Human) | APGKGLEYIGRFTISKTSTTVDLKFTSPTTEDTA | |
| Variable Heavy Chain | TYFCARWGPGTLVTVSS | |
| ST-3 Monoclonal | DPVMTQTPASVSEPVGGTVTIKCQASQSIGSSL | SEQ. ID. NO.: 9 |
| Antibody (Human) | AWYQQKPGQRPKLLIYQASKLASGVPSRFKGS | |
| Full Light Chain | RSGTEFTLTISDLECADAATYYCQCTGNGGDFI | |
| | GAFGGGTEVVVKRTVAAPSVFIFPPSDEQLKSG | |
| | TASVVCLLNNFYPREAKVQWKVDNALQSGNS | |
| | QESVTEQDSKDSTYSLSSTLTLSKADYEKHKV | |
| | YACEVTHQGLSSPVTKSFNRGEC | |
| ST-3 Monoclonal | QSLEESGGGLVTPGTPLTLTCTASGFSLSSYYV | SEQ. ID. NO.: 10 |
| Antibody (Human) | RWVRQAPGKGLEYIGIISDSGSTYYASWAKGR | |
| Full Heavy Chain | FTISKTSTTVDLKFTSPTTEDTATYFCARGSGYT | |
| | IPTDLWGPGTLVTVSSASTKGPSVFPLAPSSKST | |
| | SGGTAALGCLVKDYFPEPVTVSWNSGALTSGV | |
| | HTFPAVLQSSGLYSLSSVVTVPSSSLGTQTYICN | |
| | VNHKPSNTKVDKKVEPKSCDKTHTCPPCPAPE | |
| | LLGGPSVFLFPPKPKDTLMISRTPEVTCVVVDV | |
| | SHEDPEVKFNWYVDGVEVHNAKTKPREEQYN | |
| | STYRVVSVLTVLHQDWLNGKEYKCKVSNKAL | |
| | PAPIEKTISKAKGQPREPQVYTLPPSRDELTKNQ | |
| | VSLTCLVKGFYPSDIAVEWESNGQPENNYKTT | |
| | PPVLDSDGSFFLYSKLTVDKSRWQQGNVFSCS | |
| | VMHEALHNHYTQKSLSLSPGK | |
| ST-4 Monoclonal | RSSESLVYSNGKSYLS | SEQ. ID. NO.: 11 |
| Antibody (Human) | | |
| Light Chain CDR 1 | | |
| ST-4 Monoclonal | EVSKRDS | SEQ. ID. NO.: 12 |
| Antibody (Human) | | |
| Light Chain CDR 2 | | |
| ST-4 Monoclonal | MQGTYWPPIT | SEQ. ID. NO.: 13 |
| Antibody (Human) | | |
| Light Chain CDR 3 | | |
| ST-4 Monoclonal | LHYMH | SEQ. ID. NO.: 14 |
| Antibody (Human) | | |
| Heavy Chain CDR 4 | | |
| ST-4 Monoclonal | RISNDGSEGWYAESVKG | SEQ. ID. NO.: 15 |
| Antibody (Human) | | |
| Heavy Chain CDR 5 | | |
| ST-4 Monoclonal | DPDTSNKIDY | SEQ. ID. NO.: 16 |
| Antibody (Human) | | |
| Heavy Chain CDR 6 | | |
| ST-4 Monoclonal | DVVMTQSPLSLPVTLGQPASISCWFQQRPGQSP | SEQ. ID. NO.: 17 |
| Antibody (Human) | RRLLYGVPDKFSGSGSGTDFTLKISRVEAEDVG | |
| Variable Light Chain | VYYCFGQGTRLEIK | |
| ST-4 Monoclonal | DVVMTQSPLSLPVTLGQPASISCWFQQRPGQSP | SEQ. ID. NO.: 18 |
| Antibody (Human) | RRLLYGVPDKFSGSGSGTDFTLKISRVEAEDVG | |
| Variable Heavy Chain | VYYCFGQGTRLEIK | |
| ST-4 Monoclonal | DVVMTQSPLSLPVTLGQPASISCRSSESLVYSN | SEQ. ID. NO.: 19 |
| Antibody (Human) | GKSYLSWFQQRPGQSPRRLLYEVSKRDSGVPD | |
| Full Light Chain | KFSGSGSGTDFTLKISRVEAEDVGVYYCMQGT | |
| | YWPPITFGQGTRLEIKRTVAAPSVFIFPPSDEQL | |
| | KSGTASVVCLLNNFYPREAKVQWKVDNALQS | |
| | GNSQESVTEQDSKDSTYSLSSTLTLSKADYEKH | |
| | KVYACEVTHQGLSSPVTKSFNRGEC. | |
| ST-4 Monoclonal | QVQLVESGGDVVQPGGSLRLSCAASGFTFNLH | SEQ. ID. NO.: 20 |
| Antibody (Human) | YMHWVRQAPGRGLEWVSRISNDGSEGWYAES | |
| Full Heavy Chain | VKGRFTISRDNSKNSLYLQMNSLRAEDTAVYY | |
| | CARDPDTSNKIDYWGQGTLVTVSSASTKGPSV | |
| | FPLAPSSKSTSGGTAALGCLVKDYFPEPVTVSW | |
| | NSGALTSGVHTFPAVLQSSGLYSLSSVVTVPSS | |
| | SLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAK | |
| | TKPREEQYNSTYRVVSVLTVLHQDWLNGKEY | |
| | KCKVSNKALPAPIEKTISKAKGQPREPQVYTLP | |
| | PSRDELTKNQVSLTCLVKGFYPSDIAVEWESNG | |
| | QPENNYKTTPPVLDSDGSFFLYSKLTVDKSRW | |
| | QQGNVFSCSVMHEALHNHYTQKSLSLSPGK. | |
| ST-5 Monoclonal | QASQNTDIRLA | SEQ. ID. NO.: 21 |
| Antibody (Rabbit) | | |
| Light Chain CDR 1 | | |
| ST-5 Monoclonal | SASTLAS | SEQ. ID. NO.: 22 |
| Antibody (Rabbit) | | |
| Light Chain CDR 2 | | |
| ST-5 Monoclonal | DDAATYYCLGNYDCSYADCYA | SEQ. ID. NO.: 23 |
| Antibody (Rabbit) | | |
| Light Chain CDR 3 | | |
| ST-5 Monoclonal | NYEMG | SEQ. ID. NO.: 24 |
| Antibody (Rabbit) | | |
| Heavy Chain CDR 4 | | |
| ST-5 Monoclonal | YIRTGGSAYYASWAKG | SEQ. ID. NO.: 25 |
| Antibody (Rabbit) | | |
| Heavy Chain CDR 5 | | |
| ST-5 Monoclonal | PYAFVSLINDL | SEQ. ID. NO.: 26 |
| Antibody (Rabbit) | | |
| Heavy Chain CDR 6 | | |
| ST-5 Monoclonal | AQVLTQTPSSVSAAVGGTVTINCWYQQKPGQP | SEQ. ID. NO.: 27 |
| Antibody (Rabbit) | PKRLIYGVPSRFKGSGSGTQFTLTISDLECFGGG | |
| Variable Light Chain | TEVVVR | |
| ST-5 Monoclonal | QSLEESGGRLVTPGTPLTLTCTVSGIDLNWVRQ | SEQ. ID. NO.: 28 |
| Antibody (Rabbit) | APGKGLEWIGRFTISKTSTTVDLKMTSLATEDT | |
| Variable Heavy Chain | ATYFCARWGPGTLVTVSS | |
| ST-5 Monoclonal | AQVLTQTPSSVSAAVGGTVTINCQASQNTDIRL | SEQ. ID. NO.: 29 |
| Antibody (Rabbit) | AWYQQKPGQPPKRLIYSASTLASGVPSRFKGS | |
| Full Light Chain | GSGTQFTLTISDLECDDAATYYCLGNYDCSYA | |
| | DCYAFGGGTEVVVRRTVAAPSVFIFPPSDEQLK | |
| | SGTASVVCLLNNFYPREAKVQWKVDNALQSG | |
| | NSQESVTEQDSKDSTYSLSSTLTLSKADYEKHK | |
| | VYACEVTHQGLSSPVTKSFNRGEC | |
| ST-5 Monoclonal | QSLEESGGRLVTPGTPLTLTCTVSGIDLNNYEM | SEQ. ID. NO.: 30 |
| Antibody (Rabbit) | GWVRQAPGKGLEWIGYIRTGGSAYYASWAKG | |
| Full Heavy Chain | RFTISKTSTTVDLKMTSLATEDTATYFCARPYA | |
| | FVSLINDLWGPGTLVTVSSASTKGPSVFPLAPSS | |
| | KSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPC | |
| | PAPELLGGPSVFLFPPKPKDTLMISRTPEVTCVV | |
| | VDVSHEDPEVKFNWYVDGVEVHNAKTKPREE | |
| | QYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNY | |
| | KTTPPVLDSDGSFFLYSKLTVDKSRWQQGNVF | |
| | SCSVMHEALHNHYTQKSLSLSPGK | |
| ST-6A Monoclonal | QASQSVWKNNYLS | SEQ. ID. NO.: 31 |
| Antibody (Rabbit) | | |
| Light Chain CDR 1 | | |
| ST-6A Monoclonal | TASSLAS | SEQ. ID. NO.: 32 |
| Antibody (Rabbit) | | |
| Light Chain CDR 2 | | |
| ST-6A Monoclonal | AGDVGGGIRT | SEQ. ID. NO.: 33 |
| Antibody (Rabbit) | | |
| Light Chain CDR 3 | | |
| ST-6A Monoclonal | SYTTS | SEQ. ID. NO.: 34 |
| Antibody (Rabbit) | | |
| Heavy Chain CDR 4 | | |
| ST-6A Monoclonal | VIDVGSDDTYYATWAKG | SEQ. ID. NO.: 35 |
| Antibody (Rabbit) | | |
| Heavy Chain CDR 5 | | |
| ST-6A Monoclonal | HGATGGTVFDL | SEQ. ID. NO.: 36 |
| Antibody (Rabbit) | | |
| Heavy Chain CDR 6 | | |
| ST-6A Monoclonal | AQVLTQTPSPVSAAVGGTVTINCWFQQKPGQP | SEQ. ID. NO.: 37 |
| Antibody (Rabbit) | PKLLIYGVPSRFKGSGSGTQFTLTISDLECDDAA | |
| Variable Light Chain | TYYCFGGGTEVVVK | |
| ST-6A Monoclonal | QSLEESGGRLVTPGTPLTLTCTASGFSLSWVRQ | SEQ. ID. NO.: 38 |
| Antibody (Rabbit) | APGKGLEWVGRFTISRTSTTVDLKMTSLTAAD | |
| Variable Heavy Chain | TATYFCTRWGPGTLVTVSS | |
| ST-6A Monoclonal | AQVLTQTPSPVSAAVGGTVTINCQASQSVWKN | SEQ. ID. NO.: 39 |
| Antibody (Rabbit) | NYLSWFQQKPGQPPKLLIYTASSLASGVPSRFK | |
| Full Light Chain | GSGSGTQFTLTISDLECDDAATYYCAGDVGGGI | |
| | RTFGGGTEVVVKRTVAAPSVFIFPPSDEQLKSG | |
| | TASVVCLLNNFYPREAKVQWKVDNALQSGNS | |
| | QESVTEQDSKDSTYSLSSTLTLSKADYEKHKV | |
| | YACEVTHQGLSSPVTKSFNRGEC | |
| ST-6A Monoclonal | QSLEESGGRLVTPGTPLTLTCTASGFSLSSYTTS | SEQ. ID. NO.: 40 |
| Antibody (Rabbit) | WVRQAPGKGLEWVGVIDVGSDDTYYATWAK | |
| Full Heavy Chain | GRFTISRTSTTVDLKMTSLTAADTATYFCTRHG | |
| | ATGGTVFDLWGPGTLVTVSSASTKGPSVFPLAP | |
| | SSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCP | |
| | PCPAPELLGGPSVFLFPPKPKDTLMISRTPEVTC | |
| | VVVDVSHEDPEVKFNWYVDGVEVHNAKTKPR | |
| | EEQYNSTYRVVSVLTVLHQDWLNGKEYKCKV | |
| | SNKALPAPIEKTISKAKGQPREPQVYTLPPSRDE | |
| | LTKNQVSLTCLVKGFYPSDIAVEWESNGQPEN | |
| | NYKTTPPVLDSDGSFFLYSKLTVDKSRWQQGN | |
| | VFSCSVMHEALHNHYTQKSLSLSPGK | |
| ST-6B Monoclonal | TGTSSDVGGYNYVS | SEQ. ID. NO.: 41 |
| Antibody (Human) | | |
| Light Chain CDR 1 | | |
| ST-6B Monoclonal | EVSKRPS | SEQ. ID. NO.: 42 |
| Antibody (Human) | | |
| Light Chain CDR 2 | | |
| ST-6B Monoclonal | SSHAGSKNVI | SEQ. ID. NO.: 43 |
| Antibody (Human) | | |
| Light Chain CDR 3 | | |
| ST-6B Monoclonal | GHYMS | SEQ. ID. NO.: 44 |
| Antibody (Human) | | |
| Heavy Chain CDR 4 | | |
| ST-6B Monoclonal | KMNQDGSSRSYVDSVKG | SEQ. ID. NO.: 45 |
| Antibody (Human) | | |
| Heavy Chain CDR 5 | | |
| ST-6B Monoclonal | EEWYRFDY | SEQ. ID. NO.: 46 |
| Antibody (Human) | | |
| Heavy Chain CDR 6 | | |
| ST-6B Monoclonal | QSALTQPPSASGSPGQSVTISCTGTSSDVGGYN | SEQ. ID. NO.: 47 |
| Antibody (Human) | YVSWYRQHPGKAPKLMIYEVSKRPSGVPDRFS | |
| Variable Light Chain | GSKSGNTASLTVSGLQADDEGDYYCSSHAGSK | |
| | NVIFGGGTKVTVL | |
| ST-6B Monoclonal | EVQLVESGGGLVQPGGSLRLSCAASGFAFSGH | SEQ. ID. NO.: 48 |
| Antibody (Human) | YMSWVRQAPGKGLEWVAKRSYVDSVKGRFTI | |
| Variable Heavy Chain | SRDNAKNSLYLQMNSLRAEDTAVYYCTKEEW | |
| | YRFDYWGQGTLVTVSS | |
| ST-6B Monoclonal | QSALTQPPSASGSPGQSVTISCTGTSSDVGGYN | SEQ. ID. NO.: 49 |
| Antibody (Human) | YVSWYRQHPGKAPKLMIYEVSKRPSGVPDRFS | |
| Full Light Chain | GSKSGNTASLTVSGLQADDEGDYYCSSHAGSK | |
| | NVIFGGGTKVTVLGQPKAAPSVTLFPPSSEELQ | |
| | ANKATLVCLISDFYPGAVTVAWKADSSPVKAG | |
| | VETTTPSKQSNNKYAASSYLSLTPEQWKSHRS | |
| | YSCQVTHEGSTVEKTVAPTECS | |
| ST-6B Monoclonal | EVQLVESGGGLVQPGGSLRLSCAASGFAFSGH | SEQ. ID. NO.: 50 |
| Antibody (Human) | YMSWVRQAPGKGLEWVAKMNQDGSSRSYVD | |
| Full Heavy Chain | SVKGRFTISRDNAKNSLYLQMNSLRAEDTAVY | |
| | YCTKEEWYRFDYWGQGTLVTVSSASTKGPSVF | |
| | PLAPSSKSTSGGTAALGCLVKDYFPEPVTVSW | |
| | NSGALTSGVHTFPAVLQSSGLYSLSSVVTVPSS | |
| | SLGTQTYICNVNHKPSNTKVDKKVEPKSCDKT | |
| | HTCPPCPAPELLGGPSVFLFPPKPKDTLMISRTP | |
| | EVTCVVVDVSHEDPEVKFNWYVDGVEVHNAK | |
| | TKPREEQYNSTYRVVSVLTVLHQDWLNGKEY | |
| | KCKVSNKALPAPIEKTISKAKGQPREPQVYTLP | |
| | PSRDELTKNQVSLTCLVKGFYPSDIAVEWESNG | |
| | QPENNYKTTPPVLDSDGSFFLYSKLTVDKSRW | |
| | QQGNVFSCSVMHEALHNHYTQKSLSLSPGK | |
| ST-7F Monoclonal | TGNSNNVGNQGAA | SEQ. ID. NO.: 51 |
| Antibody (Human) | | |
| Light Chain CDR 1 | | |
| ST-7F Monoclonal | RNNNRPS | SEQ. ID. NO.: 52 |
| Antibody (Human) | | |
| Light Chain CDR 2 | | |
| ST-7F Monoclonal | SAWDSSLNAWV | SEQ. ID. NO.: 53 |
| Antibody (Human) | | |
| Light Chain CDR 3 | | |
| ST-7F Monoclonal | NYVMH | SEQ. ID. NO.: 54 |
| Antibody (Human) | | |
| Heavy Chain CDR 4 | | |
| ST-7F Monoclonal | IWSDGSTIFHADSVKG | SEQ. ID. NO.: 55 |
| Antibody (Human) | | |
| Heavy Chain CDR 5 | | |
| ST-7F Monoclonal | EPRAIADNYYGMDV | SEQ. ID. NO.: 56 |
| Antibody (Human) | | |
| Heavy Chain CDR 6 | | |
| ST-7F Monoclonal | QAGLTQPPSVSKGLRQTATLTCWLQQHQGHPP | SEQ. ID. NO.: 57 |
| Antibody (Human) | KLLSYGISERLSASRSGNTASLTITGLQPEDEAD | |
| Variable Light Chain | YYCFGGGTKLAVL | |
| ST-7F Monoclonal | QVQLVESGGDVVQPGRSLRLSCAASGFTFSWV | SEQ. ID. NO.: 58 |
| Antibody (Human) | RQAPGEGLEWVSLRFTISRDNSKNTLYLQMDS | |
| Variable Heavy Chain | LRAEDTAVYYCARWGQGTSVTVSS | |
| ST-7F Monoclonal | QAGLTQPPSVSKGLRQTATLTCTGNSNNVGNQ | SEQ. ID. NO.: 59 |
| Antibody (Human) | GAAWLQQHQGHPPKLLSYRNNNRPSGISERLS | |
| Full Light Chain | ASRSGNTASLTITGLQPEDEADYYCSAWDSSLN | |
| | AWVFGGGTKLAVLGQPKAAPSVTLFPPSSEEL | |
| | QANKATLVCLISDFYPGAVTVAWKADSSPVKA | |
| | GVETTTPSKQSNNKYAASSYLSLTPEQWKSHR | |
| | SYSCQVTHEGSTVEKTVAPTECS | |
| ST-7F Monoclonal | QVQLVESGGDVVQPGRSLRLSCAASGFTFSNY | SEQ. ID. NO.: 60 |
| Antibody (Human) | VMHWVRQAPGEGLEWVSLIWSDGSTIFHADS | |
| Full Heavy Chain | VKGRFTISRDNSKNTLYLQMDSLRAEDTAVYY | |
| | CAREPRAIADNYYGMDVWGQGTSVTVSSAST | |
| | KGPSVFPLAPSSKSTSGGTAALGCLVKDYFPEP | |
| | VTVSWNSGALTSGVHTFPAVLQSSGLYSLSSV | |
| | VTVPSSSLGTQTYICNVNHKPSNTKVDKKVEP | |
| | KSCDKTHTCPPCPAPELLGGPSVFLFPPKPKDTL | |
| | MISRTPEVTCVVVDVSHEDPEVKFNWYVDGVE | |
| | VHNAKTKPREEQYNSTYRVVSVLTVLHQDWL | |
| | NGKEYKCKVSNKALPAPIEKTISKAKGQPREPQ | |
| | VYTLPPSRDELTKNQVSLTCLVKGFYPSDIAVE | |
| | WESNGQPENNYKTTPPVLDSDGSFFLYSKLTV | |
| | DKSRWQQGNVFSCSVMHEALHNHYTQKSLSL | |
| | SPGK | |
| ST-9V Monoclonal | QASKTVYDDNALA | SEQ. ID. NO.: 61 |
| Antibody (Rabbit) | | |
| Light Chain CDR 1 | | |
| ST-9V Monoclonal | KASTLAS | SEQ. ID. NO.: 62 |
| Antibody (Rabbit) | | |
| Light Chain CDR 2 | | |
| ST-9V Monoclonal | AGGYIYDSGDHA | SEQ. ID. NO.: 63 |
| Antibody (Rabbit) | | |
| Light Chain CDR 3 | | |
| ST-9V Monoclonal | RGQVG | SEQ. ID. NO.: 64 |
| Antibody (Rabbit) | | |
| Heavy Chain CDR 4 | | |
| ST-9V Monoclonal | FKGYGGNAFYTNWAKG | SEQ. ID. NO.: 65 |
| Antibody (Rabbit) | | |
| Heavy Chain CDR 5 | | |
| ST-9V Monoclonal | VAGDINHLDL | SEQ. ID. NO.: 66 |
| Antibody (Rabbit) | | |
| Heavy Chain CDR 6 | | |
| ST-9V Monoclonal | AAVLTQTPSPVSAAVGGTVSINCWYQQKPGQP | SEQ. ID. NO.: 67 |
| Antibody (Rabbit) | PKLLIYGVPSRFSGSGSGTQFTLTISDVQCDDAA | |
| Variable Light Chain | TYYCFGGGTEVVV | |
| ST-9V Monoclonal | QSLEESGGRLVTPGTPLTLTCTVSGIDLSWVRQ | SEQ. ID. NO.: 68 |
| Antibody (Rabbit) | APGEGLEYIGRFTISKTSSTTVDLKITTPTTEDTA | |
| Variable Heavy Chain | TYFCARWGQGTLVTVSS | |
| ST-9V Monoclonal | AAVLTQTPSPVSAAVGGTVSINCQASKTVYDD | SEQ. ID. NO.: 69 |
| Antibody (Rabbit) | NALAWYQQKPGQPPKLLIYKASTLASGVPSRF | |
| Full Light Chain | SGSGSGTQFTLTISDVQCDDAATYYCAGGYIY | |
| | DSGDHAFGGGTEVVVKRTVAAPSVFIFPPSDEQ | |
| | LKSGTASVVCLLNNFYPREAKVQWKVDNALQ | |
| | SGNSQESVTEQDSKDSTYSLSSTLTLSKADYEK | |
| | HKVYACEVTHQGLSSPVTKSFNRGEC | |
| ST-9V Monoclonal | QSLEESGGRLVTPGTPLTLTCTVSGIDLSRGQV | SEQ. ID. NO.: 70 |
| Antibody (Rabbit) | GWVRQAPGEGLEYIGFKGYGGNAFYTNWAKG | |
| Full Heavy Chain | RFTISKTSSTTVDLKITTPTTEDTATYFCARVAG | |
| | DINHLDLWGQGTLVTVSSASTKGPSVFPLAPSS | |
| | KSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPC | |
| | PAPELLGGPSVFLFPPKPKDTLMISRTPEVTCVV | |
| | VDVSHEDPEVKFNWYVDGVEVHNAKTKPREE | |
| | QYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNY | |
| | KTTPPVLDSDGSFFLYSKLTVDKSRWQQGNVF | |
| | SCSVMHEALHNHYTQKSLSLSPGK | |
| ST-11A Monoclonal | QASQSIGSYLA | SEQ. ID. NO.: 71 |
| Antibody (Rabbit) | | |
| Light Chain CDR 1 | | |
| ST-11A Monoclonal | YVFKVAP | SEQ. ID. NO.: 72 |
| Antibody (Rabbit) | | |
| Light Chain CDR 2 | | |
| ST-11A Monoclonal | ASYAGSSSSA | SEQ. ID. NO.: 73 |
| Antibody (Rabbit) | | |
| Light Chain CDR 3 | | |
| ST-11A Monoclonal | IYAVG | SEQ. ID. NO.: 74 |
| Antibody (Rabbit) | | |
| Heavy Chain CDR 4 | | |
| ST-11A Monoclonal | TISTVDRSYYATWAKG | SEQ. ID. NO.: 75 |
| Antibody (Rabbit) | | |
| Heavy Chain CDR 5 | | |
| ST-11A Monoclonal | GLSCSNTDCAFNI | SEQ. ID. NO.: 76 |
| Antibody (Rabbit) | | |
| Heavy Chain CDR 6 | | |
| ST-11A Monoclonal | AFELTQTPSSVEAAVGGTVTISCQASQSIGSYLA | SEQ. ID. NO.: 77 |
| Antibody (Rabbit) | WYQQKPGQPPKLLIYYVFKVAPGVPSRFSGSG | |
| Variable Light Chain | SGTQFTLTISDLECADAATYYCASYAGSSSSAF | |
| | GGGTEVVVK | |
| ST-11A Monoclonal | QSVEESGGRLVTPGTPLTLTCTVSGIDLSIYAVG | SEQ. ID. NO.: 78 |
| Antibody (Rabbit) | WVRQAPGKGLEYIGTISTVDRSYYATWAKGRF | |
| Variable Heavy Chain | TISKTSTTVDLKITSPTTEDTATYFCGRGLSCSN | |
| | TDCAFNIWGPGTLVTVS | |
| ST-11A Monoclonal | AFELTQTPSSVEAAVGGTVTISCQASQSIGSYLA | SEQ. ID. NO.: 79 |
| Antibody (Rabbit) | WYQQKPGQPPKLLIYYVFKVAPGVPSRFSGSG | |
| Full Light Chain | SGTQFTLTISDLECADAATYYCASYAGSSSSAF | |
| | GGGTEVVVKRTVAAPSVFIFPPSDEQLKSGTAS | |
| | VVCLLNNFYPREAKVQWKVDNALQSGNSQES | |
| | VTEQDSKDSTYSLSSTLTLSKADYEKHKVYAC | |
| | EVTHQGLSSPVTKSFNRGEC | |
| ST-11A Monoclonal | QSVEESGGRLVTPGTPLTLTCTVSGIDLSIYAVG | SEQ. ID. NO.: 80 |
| Antibody (Human) | WVRQAPGKGLEYIGTISTVDRSYYATWAKGRF | |
| Full Heavy Chain | TISKTSTTVDLKITSPTTEDTATYFCGRGLSCSN | |
| | TDCAFNIWGPGTLVTVSSASTKGPSVFPLAPSS | |
| | KSTSGGTAALGCLVKDYFPEPVTVSWNSGALT | |
| | SGVHTFPAVLQSSGLYSLSSVVTVPSSSLGTQT | |
| | YICNVNHKPSNTKVDKKVEPKSCDKTHTCPPC | |
| | PAPELLGGPSVFLFPPKPKDTLMISRTPEVTCVV | |
| | VDVSHEDPEVKFNWYVDGVEVHNAKTKPREE | |
| | QYNSTYRVVSVLTVLHQDWLNGKEYKCKVSN | |
| | KALPAPIEKTISKAKGQPREPQVYTLPPSRDELT | |
| | KNQVSLTCLVKGFYPSDIAVEWESNGQPENNY | |
| | KTTPPVLDSDGSFFLYSKLTVDKSRWQQGNVF | |
| | SCSVMHEALHNHYTQKSLSLSPGK | |
| ST-14 Monoclonal | DVLMTQTPLSLPVSLGDQASIFCRSSQSIVYSDG | SEQ. ID. NO.: 81 |
| Antibody (Mouse) | NTYLEWYLQKPGQSPKLLIYKVSHRFSGVPDR | |
| Variable Light Chain | FSGSGSGTDFTLKISRVEAEDLGVYFCFQGSHV | |
| | PWTFGGGTKLEIK | |
| ST-14 Monoclonal | EVQLQQSGPGLVKPGASVKMSCKASGYTFTDY | SEQ. ID. NO.: 82 |
| Antibody (Mouse) | YMKWMKQSHGKSLEWIGDINPNNYDTNYNQ | |
| Variable Heavy Chain | KFKGRATLTVDKSSSTAYMQLNSLTSEDSAVY | |
| | YCARWDYWGQGTTLT | |
| ST-14 Monoclonal | DVLMTQTPLSLPVSLGDQASIFCRSSQSIVYSDG | SEQ. ID. NO.: 83 |
| Antibody (Mouse) | NTYLEWYLQKPGQSPKLLIYKVSHRFSGVPDR | |
| Full Light Chain | FSGSGSGTDFTLKISRVEAEDLGVYFCFQGSHV | |
| | PWTFGGGTKLEIKRADAAPTVSIFPPSSEQLTSG | |
| | GASVVCFLNNFYPKDINVKWKIDGSERQNGVL | |
| | NSWTDQDSKDSTYSMSSTLTLTKDEYERHNSY | |
| | TCEATHKTSTSPIVKSFNRNEC | |
| ST-14 Monoclonal | EVQLQQSGPGLVKPGASVKMSCKASGYTFTDY | SEQ. ID. NO.: 84 |
| Antibody (Mouse) | YMKWMKQSHGKSLEWIGDINPNNYDTNYNQ | |
| Full Heavy Chain | KFKGRATL | |
| | TVDKSSSTAYMQLNSLTSEDSAVYYCARWDY | |
| | WGQGTTLTVSSAKTTPPSVYPLAPGSAAQTNS | |
| | MVTLGCLVKGYFPEPVTVTWNSGSLSSGVHTF | |
| | PAVLQSDLYTLSSSVTVPSSTWPSETVTCNVAH | |
| | PASSTKVDKKIVPRDCGCKPCICTVPEVSSVFIF | |
| | PPKPKDVLTITLTPKVTCVVVDISKDDPEVQFS | |
| | WFVDDVEVHTAQTQPREEQFNSTFRSVSELPIM | |
| | HQDWLNGKEFKCRVNSAAFPAPIEKTISKTKG | |
| | RPKAPQVYTIPPPKEQMAKDKVSLTCMITDFFP | |
| | EDITVEWQWNGQPAENYKNTQPIMDTDGSYF | |
| | VYSKLNVQKSNWEAGNTFTCSVLHEGLHNHH | |
| | TEKSLSHSPGK | |
| ST-19A Monoclonal | QASQSVGGNNALS | SEQ. ID. NO.: 85 |
| Antibody (Rabbit) | | |
| Light Chain CDR 1 | | |
| ST-19A Monoclonal | GASTLAS | SEQ. ID. NO.: 86 |
| Antibody (Rabbit) | | |
| Light Chain CDR 2 | | |
| ST-19A Monoclonal | LGGYGGIGDNA | SEQ. ID. NO.: 87 |
| Antibody (Rabbit) | | |
| Light Chain CDR 3 | | |
| ST-19A Monoclonal | TYNIC | SEQ. ID. NO.: 88 |
| Antibody (Rabbit) | | |
| Heavy Chain CDR 4 | | |
| ST-19A Monoclonal | CINTGGSAFYTTWVKG | SEQ. ID. NO.: 89 |
| Antibody (Rabbit) | | |
| Heavy Chain CDR 5 | | |
| ST-19A Monoclonal | YVDGTGYWGTRLDL | SEQ. ID. NO.: 90 |
| Antibody (Rabbit) | | |
| Heavy Chain CDR 6 | | |
| ST-19A Monoclonal | AAVLTQTPSPVSAAVGGTVTISCQASQSVGGN | SEQ. ID. NO.: 91 |
| Antibody (Rabbit) | NALSWFQQKPGQPPKLLIYGASTLASGVPSRFS | |
| Variable Light Chain | ASGSGTQFTLTISDVQCDDAATYYCLGGYGGI | |
| | GDNAFGGGTEVVVK | |
| ST-19A Monoclonal | QSVEESGGRLVTPGTPLTLTCTASGFSLSTYNIC | SEQ. ID. NO.: 92 |
| Antibody (Rabbit) | WVRQSPGKGLEYIGCINTGGSAFYTTWVKGRF | |
| Variable Heavy Chain | TISKASTTVDLRITSPTTEDTAIYFCSSYVDGTG | |
| | YWGTRLDLWGQGTLVTVSS | |
| ST-19A Monoclonal | AAVLTQTPSPVSAAVGGTVTISCQASQSVGGN | SEQ. ID. NO.: 93 |
| Antibody (Rabbit) | NALSWFQQKPGQPPKLLIYGASTLASGVPSRFS | |
| Full Light Chain | ASGSGTQFTLTISDVQCDDAATYYCLGGYGGI | |
| | GDNAFGGGTEVVVKRTVAAPSVFIFPPSDEQLK | |
| | SGTASVVCLLNNFYPREAKVQWKVDNALQSG | |
| | NSQESVTEQDSKDSTYSLSSTLTLSKADYEKHK | |
| | VYACEVTHQGLSSPVTKSFNRGEC | |
| ST-19A Monoclonal | QSVEESGGRLVTPGTPLTLTCTASGFSLSTYNIC | SEQ. ID. NO.: 94 |
| Antibody (Rabbit) | WVRQSPGKGLEYIGCINTGGSAFYTTWVKGRF | |
| Full Heavy Chain | TISKASTTVDLRITSPTTEDTAIYFCSSYVDGTG | |
| | YWGTRLDLWGQGTLVTVSSASTKGPSVFPLAP | |
| | SSKSTSGGTAALGCLVKDYFPEPVTVSWNSGA | |
| | LTSGVHTFPAVLQSSGLYSLSSVVTVPSSSLGT | |
| | QTYICNVNHKPSNTKVDKKVEPKSCDKTHTCP | |
| | PCPAPELLGGPSVFLFPPKPKDTLMISRTPEVTC | |
| | VVVDVSHEDPEVKFNWYVDGVEVHNAKTKPR | |
| | EEQYNSTYRVVSVLTVLHQDWLNGKEYKCKV | |
| | SNKALPAPIEKTISKAKGQPREPQVYTLPPSRDE | |
| | LTKNQVSLTCLVKGFYPSDIAVEWESNGQPEN | |
| | NYKTTPPVLDSDGSFFLYSKLTVDKSRWQQGN | |
| | VFSCSVMHEALHNHYTQKSLSLSPGK | |
| ST-22F Monoclonal | QSSQSVYGNNWLA | SEQ. ID. NO.: 95 |
| Antibody (Rabbit) | | |
| Light Chain CDR 1 | | |
| ST-22F Monoclonal | DASELAS | SEQ. ID. NO.: 96 |
| Antibody (Rabbit) | | |
| Light Chain CDR 2 | | |
| ST-22F Monoclonal | QGGFIGSDRHG | SEQ. ID. NO.: 97 |
| Antibody (Rabbit) | | |
| Light Chain CDR 3 | | |
| ST-22F Monoclonal | SYYYMC | SEQ. ID. NO.: 98 |
| Antibody (Rabbit) | | |
| Heavy Chain CDR 4 | | |
| ST-22F Monoclonal | CIYAGNADSTYYATWAKG | SEQ. ID. NO.: 99 |
| Antibody (Rabbit) | | |
| Heavy Chain CDR 5 | | |
| ST-22F Monoclonal | NYYAGGTAGYAHSAFDP | SEQ. ID. NO.: 100 |
| Antibody (Rabbit) | | |
| Heavy Chain CDR 6 | | |
| ST-22F Monoclonal | AAVLTQTPSPVSVAVGGTVTINCWYQQKPGQP | SEQ. ID. NO.: 101 |
| Antibody (Rabbit) | PKLLIYGVPSRFSGSGYGTEFTLTISGVQCEDAA | |
| Variable Light Chain | TYYCFGGGTEVVVK | |
| ST-22F Monoclonal | QQLEESGGGLVKPGGTLTLTCKASGIDFSWVR | SEQ. ID. NO.: 102 |
| Antibody (Rabbit) | QAPGKGLEWVGRFTMSKTSSTTVTLQMTSLTS | |
| Variable Heavy Chain | ADTATYFCARWGPGTPVTVSS | |
| ST-22F Monoclonal | AAVLTQTPSPVSVAVGGTVTINCQSSQSVYGN | SEQ. ID. NO.: 103 |
| Antibody (Rabbit) | NWLAWYQQKPGQPPKLLIYDASELASGVPSRF | |
| Full Light Chain | SGSGYGTEFTLTISGVQCEDAATYYCQGGFIGS | |
| | DRHGFGGGTEVVVKRTVAAPSVFIFPPSDEQLK | |
| | SGTASVVCLLNNFYPREAKVQWKVDNALQSG | |
| | NSQESVTEQDSKDSTYSLSSTLTLSKADYEKHK | |
| | VYACEVTHQGLSSPVTKSFNRGEC | |
| ST-22F Monoclonal | QQLEESGGGLVKPGGTLTLTCKASGIDFSSYYY | SEQ. ID. NO.: 104 |
| Antibody (Rabbit) | MCWVRQAPGKGLEWVGCIYAGNADSTYYAT | |
| Full Heavy Chain | WAKGRFTMSKTSSTTVTLQMTSLTSADTATYF | |
| | CARNYYAGGTAGYAHSAFDPWGPGTPVTVSS | |
| | ASTKGPSVFPLAPSSKSTSGGTAALGCLVKDYF | |
| | PEPVTVSWNSGALTSGVHTFPAVLQSSGLYSLS | |
| | SVVTVPSSSLGTQTYICNVNHKPSNTKVDKKV | |
| | EPKSCDKTHTCPPCPAPELLGGPSVFLFPPKPKD | |
| | TLMISRTPEVTCVVVDVSHEDPEVKFNWYVDG | |
| | VEVHNAKTKPREEQYNSTYRVVSVLTVLHQD | |
| | WLNGKEYKCKVSNKALPAPIEKTISKAKGQPR | |
| | EPQVYTLPPSRDELTKNQVSLTCLVKGFYPSDI | |
| | AVEWESNGQPENNYKTTPPVLDSDGSFFLYSK | |
| | LTVDKSRWQQGNVFSCSVMHEALHNHYTQKS | |
| | LSLSPGK | |
| ST-23F Monoclonal | QASESISSWLS | SEQ. ID. NO.: 105 |
| Antibody (Rabbit) | | |
| Light Chain CDR 1 | | |
| ST-23F Monoclonal | RASTLES | SEQ. ID. NO.: 106 |
| Antibody (Rabbit) | | |
| Light Chain CDR 2 | | |
| ST-23F Monoclonal | QSYARINSASYSNL | SEQ. ID. NO.: 107 |
| Antibody (Rabbit) | | |
| Light Chain CDR 3 | | |
| ST-23F Monoclonal | TYVMT | SEQ. ID. NO.: 108 |
| Antibody (Rabbit) | | |
| Heavy Chain CDR 4 | | |
| ST-23F Monoclonal | VMATDGSAYYATWTKG | SEQ. ID. NO.: 109 |
| Antibody (Rabbit) | | |
| Heavy Chain CDR 5 | | |
| ST-23F Monoclonal | GSGWESYFNT | SEQ. ID. NO.: 110 |
| Antibody (Rabbit) | | |
| Heavy Chain CDR 6 | | |
| ST-23F Monoclonal | DIVMTQTPSSASEPVGGTVTIKCQASESISSWLS | SEQ. ID. NO.: 111 |
| Antibody (Rabbit) | WYQQKPGQPPKLLIYRASTLESGVPSRFKGSGS | |
| Variable Light Chain | GTEFTLTISDLECADAATYFCQSYARINSASYS | |
| | NLFGGGTEVVVK | |
| ST-23F Monoclonal | QSVEESGGRLVTPGTPLTLTCTASGFSLSTYVM | SEQ. ID. NO.: 112 |
| Antibody (Rabbit) | TWVRQAPGKGLEWIGVMATDGSAYYATWTK | |
| Variable Heavy Chain | GRLTISRTSTTVELTITSPTTEDTATYFCARGSG | |
| | WESYFNTWGPGTLVTVSL | |
| ST-23F Monoclonal | DIVMTQTPSSASEPVGGTVTIKCQASESISSWLS | SEQ. ID. NO.: 113 |
| Antibody (Rabbit) | WYQQKPGQPPKLLIYRASTLESGVPSRFKGSGS | |
| Full Light Chain | GTEFTLTISDLECADAATYFCQSYARINSASYS | |
| | NLFGGGTEVVVKRTVAAPSVFIFPPSDEQLKSG | |
| | TASVVCLLNNFYPREAKVQWKVDNALQSGNS | |
| | QESVTEQDSKDSTYSLSSTLTLSKADYEKHKV | |
| | YACEVTHQGLSSPVTKSFNRGEC | |
| ST-23F Monoclonal | QSVEESGGRLVTPGTPLTLTCTASGFSLSTYVM | SEQ. ID. NO.: 114 |
| Antibody (Rabbit) | TWVRQAPGKGLEWIGVMATDGSAYYATWTK | |
| Full Heavy Chain | GRLTISRTSTTVELTITSPTTEDTATYFCARGSG | |
| | WESYFNTWGPGTLVTVSLASTKGPSVFPLAPSS | |
| | KSTSGGTAALGCLVKDYFPEPVTVS | |
| | WNSGALTSGVHTFPAVLQSSGLYSLSSVVTVPS | |
| | SSLGTQTYICNVNHKPSNTKVDKKVEPKSCDK | |
| | THTCPPCPAPELLGGPSVFLFPPKPKDTLMISRT | |
| | PEVTCVVVDVSHEDPEVKFNWYVDGVEVHNA | |
| | KTKPREEQYNSTYRVVSVLTVLHQDWLNGKE | |
| | YKCKVSNKALPAPIEKTISKAKGQPREPQVYTL | |
| | PPSRDELTKNQVSLTCLVKGFYPSDIAVEWESN | |
| | GQPENNYKTTPPVLDSDGSFFLYSKLTVDKSR | |
| | WQQGNVFSCSVMHEALHNHYTQKSLSLSPGK | |
| ST-33F Monoclonal | AYDMS | SEQ. ID. NO.: 115 |
| Antibody (Rabbit) | | |
| Light Chain CDR 1 | | |
| ST-33F Monoclonal | IIDTGGSAYYMNWAKG | SEQ. ID. NO.: 116 |
| Antibody (Rabbit) | | |
| Light Chain CDR 2 | | |
| ST-33F Monoclonal | VPWSSDSGSYLNL | SEQ. ID. NO.: 117 |
| Antibody (Rabbit) | | |
| Light Chain CDR 3 | | |
| ST-33F Monoclonal | QASESIGSYLS | SEQ. ID. NO.: 118 |
| Antibody (Rabbit) | | |
| Heavy Chain CDR 4 | | |
| ST-33F Monoclonal | YASTLAS | SEQ. ID. NO.: 119 |
| Antibody (Rabbit) | | |
| Heavy Chain CDR 5 | | |
| ST-33F Monoclonal | AGYKNWINDEYP | SEQ. ID. NO.: 120 |
| Antibody (Rabbit) | | |
| Heavy Chain CDR 6 | | |
| ST-33F Monoclonal | ALVMTQTPSPVSAAVGSTVTIWYQQKPGQPPK | SEQ. ID. NO.: 121 |
| Antibody (Rabbit) | LLIYGVPSRFSGSGSGTQFTLTISGVECDDAATY | |
| Variable Light Chain | YCFGGGTEVVVK | |
| ST-33F Monoclonal | QSVEESGGRLVTPGTPLTLTCTASGFSLSWVRQ | SEQ. ID. NO.: 122 |
| Antibody (Rabbit) | APGKGLEWIGRFTISRTSTAVDLKMTSLTTEDT | |
| Variable Heavy Chain | ATYFCARWGPGTLVTVSS | |
| ST-33F Monoclonal | ALVMTQTPSPVSAAVGSTVTISCQASESIGSYLS | SEQ. ID. NO.: 123 |
| Antibody (Rabbit) | WYQQKPGQPPKLLIYYASTLASGVPSRFSGSGS | |
| Full Light Chain | GTQFTLTISGVECDDAATYYCAGYKNWINDEY | |
| | PFGGGTEVVVKRTVAAPSVFIFPPSDEQLKSGT | |
| | ASVVCLLNNFYPREAKVQWKVDNALQSGNSQ | |
| | ESVTEQDSKDSTYSLSSTLTLSKADYEKHKVY | |
| | ACEVTHQGLSSPVTKSFNRGEC | |
| ST-33F Monoclonal | QSVEESGGRLVTPGTPLTLTCTASGFSLSAYDM | SEQ. ID. NO.: 124 |
| Antibody (Rabbit) | SWVRQAPGKGLEWIGIIDTGGSAYYMNWAKG | |
| Full Heavy Chain | RFTISRTSTAVDLKMTSLTTEDTATYFCARVPW | |
| | SSDSGSYLNLWGPGTLVTVSSASTKGPSVFPLA | |
| | PSSKSTSGGTAALGCLVKDYFPEPVTVSWNSG | |
| | ALTSGVHTFPAVLQSSGLYSLSSVVTVPSSSLG | |
| | TQTYICNVNHKPSNTKVDKKVEPKSCDKTHTC | |
| | PPCPAPELLGGPSVFLFPPKPKDTLMISRTPEVT | |
| | CVVVDVSHEDPEVKFNWYVDGVEVHNAKTKP | |
| | REEQYNSTYRVVSVLTVLHQDWLNGKEYKCK | |
| | VSNKALPAPIEKTISKAKGQPREPQVYTLPPSRD | |
| | ELTKNQVSLTCLVKGFYPSDIAVEWESNGQPE | |
| | NNYKTTPPVLDSDGSFFLYSKLTVDKSRWQQG | |
| | NVFSCSVMHEALHNHYTQKSLSLSPGK | |

In one aspect, the invention relates to a monoclonal antibody (mAb), or a functional variant thereof, that specifically binds to a pneumococcal serotype (ST) capsular polysaccharide, wherein said mAb comprises:
a) six complementarity determining regions (CDRs) selected from the group consisting of SEQ. ID. NOs.: 1-6, 11-16, 21-26, 31-36, 41-46, 51-56, 61-66, 71-76, 85-90, 95-100, 105-110 and 115-120;
b) a variable heavy chain and a variable light chain having an amino acid sequence selected from the group consisting of SEQ ID NOs: 7-8, 17-18, 27-28, 37-38, 47-48, 57-58, 67-68, 77-78, 81-82, 91-92, 101-102, 111-112 and 121-122; or
c) a full length light chain and a full length heavy chain having an amino acid sequence selected from the group consisting of SEQ ID NOs: 9-10, 19-20, 29-30, 39-40, 49-50, 59-60, 69-70, 79-80, 83-84, 93-94, 103-104, 113-114 and 123-124.

In one embodiment, the invention provides a monoclonal antibody comprising six CDRs selected from the group consisting of SEQ. ID. NOs.: 1-6, 11-16, 21-26, 31-36, 41-46, 51-56, 61-66, 71-76, 85-90, 95-100, 105-110 and 115-120.

In yet another embodiment, the invention provides a monoclonal antibody comprising a variable heavy chain and a variable light chain having an amino acid sequence selected from the group consisting of SEQ ID NOs: 7-8, 17-18, 27-28, 37-38, 47-48, 57-58, 67-68, 77-78, 81-82, 91-92, 101-102, 111-112 and 121-122.

In another embodiment, the invention provides a monoclonal antibody comprising a full length light chain and a full length heavy chain having an amino acid sequence selected from the group consisting of SEQ ID NOs: 9-10, 19-20, 29-30, 39-40, 49-50, 59-60, 69-70, 79-80, 83-84, 93-94, 103-104, 113-114 and 123-124.

In one embodiment functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, or at most 1 amino acid difference when compared to a corresponding reference CDR sequence, and when combined with the remaining 5 CDRs (or variants thereof) enables the variant antibody to bind to the same target antigen as the reference antibody.

In one embodiment a variant antibody comprises: a light chain CDR1 having at most 2 amino acid differences when compared to a corresponding reference CDR sequence; a light chain CDR2 having at most 2 amino acid differences when compared to a corresponding reference CDR sequence; a light chain CDR3 having at most 2 amino acid differences when compared to a corresponding reference CDR sequence; a heavy chain CDR1 having at most 2 amino acid differences when compared to a corresponding reference CDR sequence; a heavy chain CDR2 having at most 2 amino acid differences when compared to a corresponding reference CDR sequence; a heavy chain CDR3 having at most 2 amino acid differences when compared to a corresponding reference CDR sequence; wherein the variant antibody binds to the same target antigen as the reference antibody.

In some embodiments, a variant antibody comprises: a light chain CDR1 having at most 1 amino acid difference when compared to a corresponding reference CDR sequence; a light chain CDR2 having at most 1 amino acid difference when compared to a corresponding reference CDR sequence; a light chain CDR3 having at most 1 amino acid difference when compared to a corresponding reference CDR sequence; a heavy chain CDR1 having at most 1 amino acid difference when compared to a corresponding reference CDR sequence; a heavy chain CDR2 having at most 1 amino acid difference when compared to a corresponding reference CDR sequence; a heavy chain CDR3 having at most 1 amino acid difference when compared to a corresponding reference CDR sequence; wherein the variant antibody binds to the same target antigen as the reference antibody.

For example, a variant of the first antibody may comprise: a light chain CDR1 having at most 2 amino acid differences when compared to SEQ ID NO: 1; a light chain CDR2 having at most 2 amino acid differences when compared to SEQ ID NO: 2; a light chain CDR3 having at most 2 amino acid differences when compared to SEQ ID NO: 3; a light chain CDR4 having at most 2 amino acid differences when compared to SEQ ID NO: 4; a light chain CDR5 having at most 2 amino acid differences when compared to SEQ ID NO: 5; a light chain CDR6 having at most 2 amino acid differences when compared to SEQ ID NO: 6; wherein the variant antibody binds to a S. pneumoniae ST-3 capsular polysaccharide.

For example, a variant of the first antibody may comprise: a light chain CDR1 having at most 1 amino acid difference when compared to SEQ ID NO: 1; a light chain CDR2 having at most 1 amino acid difference when compared to SEQ ID NO: 2; a light chain CDR3 having at most 1 amino acid difference when compared to SEQ ID NO: 3; a light chain CDR4 having at most 1 amino acid difference when compared to SEQ ID NO: 4; a light chain CDR5 having at most 1 amino acid difference when compared to SEQ ID NO: 5; a light chain CDR6 having at most 1 amino acid difference when compared to SEQ ID NO: 6; wherein the variant antibody binds to a S. pneumoniae ST-3 capsular polysaccharide.

In one embodiment a variant antibody has at most 5, 4 or 3 amino acid differences total in the CDRs thereof when compared to a corresponding reference antibody, with the proviso that there is at most 2 (or at most 1) amino acid differences per CDR. In other embodiments, a variant antibody has at most 2 (or at most 1) amino acid differences in total in the CDRs thereof when compared to a corresponding reference antibody, with the proviso that there is at most 2 amino acid differences per CDR. In further embodiments, a variant antibody has at most 2 (or at most 1) amino acid differences total in the CDRs thereof when compared to a corresponding reference antibody, with the proviso that there is at most 1 amino acid difference per CDR. The amino acid difference may be an amino acid substitution, insertion or deletion. In one embodiment, the amino acid difference is a conservative amino acid substitution as described herein.

In one embodiment, a variant antibody has the same framework sequences as the exemplary antibodies described herein. In another embodiment the variant antibody comprises a framework region having at most 2, or at most 1 amino acid difference (when compared to a corresponding reference framework sequence). Thus, each framework region may have at most 2, or at most 1 amino acid difference (when compared to a corresponding reference framework sequence).

In one embodiment, a variant antibody has at most 5, 4 or 3 amino acid differences total in the framework regions thereof when compared to a corresponding reference antibody, with the proviso that there is at most 2 (or at most 1) amino acid differences per framework region. In some embodiments, a variant antibody has at most 2 (or at most 1) amino acid differences in total in the framework regions thereof when compared to a corresponding reference antibody, with the proviso that there is at most 2 amino acid differences per framework region. In other embodiments, a variant antibody has at most 2 (or at most 1) amino acid differences total in the framework regions thereof when compared to a corresponding reference antibody, with the proviso that there is at most 1 amino acid difference per framework region.

Thus, a variant antibody may comprise a variable light chain and a variable heavy chain as described herein, wherein: the light chain has at most 14 amino acid differences (at most 2 amino acid differences in each CDR and at most 2 amino acid differences in each framework region) when compared to a light chain sequence herein: the heavy chain has at most 14 amino acid differences (at most 2 amino acid differences in each CDR and at most 2 amino acid differences in each framework region) when compared to a heavy chain sequence herein; wherein the variant antibody binds to the same target antigen as the reference antibody.

Said variant light or heavy chains may be referred to as “functional equivalents” of the reference light and heavy chains.

In one embodiment a variant antibody comprises a variable light chain and variable heavy chain as described herein, wherein: the light chain has at most 7 amino acid differences (at most 1 amino acid differences in each CDR and at most 1 amino acid differences in each framework region) when compared to a light chain sequence herein: the heavy chain has at most 7 amino acid differences (at most 1 amino acid differences in each CDR and at most 1 amino acid differences in each framework region) when compared to a heavy chain sequence herein; wherein the variant antibody binds to the same target antigen as the reference antibody.

The present methods are further illustrated in the following non-limiting Examples.

### EXAMPLES

### General Methods of Making the Antibodies

Certain monoclonal antibodies used in methods of this invention were discovered through molecular cloning of antibody genes from plasmablast B cells post pneumococcal conjugate vaccine (PCV13) immunization (Chen et al. BMC Infect. Dis. 18:613 2018 and Cox, K. S. et al. J. Immunol. 200:180 2018).

Other monoclonal antibodies used in methods of this invention were generated through the immunization of rabbits with individual pneumococcal polysaccharides conjugated to the carrier protein, CRM197. Briefly, rabbit lymphocytes were isolated and fused with partner cells to generate multiclones and subclones that were screened and selected based on specificity for desired polysaccharide and relative binding affinity. The purified antibodies were sequenced and produced using the original rabbit backbone or substituted with a human constant region.

The antibodies used in the methods of the invention were tested in ELISA binding and specificity assays against particular serotypes.

### General Assay Procedures

The assay is performed by comparison of (i) serotype-specific binding of a serospecific antibody to its corresponding polysaccharide in a standard polysaccharide sample, with (ii) serotype-specific binding of the same antibody to its corresponding unconjugated polysaccharide that is present in a vaccine drug product. The antibody binding reactions to the polysaccharide standard and to the polysaccharide(s) present in the vaccine drug product are performed in the same fashion and analyzed on HPLC using specified chromatographic separation parameters. A typical injection volume is from 50 μL to 100 μL. The chromatograms obtained are then processed using appropriate software for peak integrations.

The methodology employed is described in detail immediately below, and in the Examples that follow.

### Standard Chromatographic Conditions

Unless otherwise indicated, size-exclusion chromatography (SEC) was carried out using HPLC, using size-exclusion columns on either an Agilent HPLC system (Agilent 1100 or 1260) (Agilent, DE, USA) or a Waters HPLC system (Waters Alliance or ARC system) (Waters Corporation, Milford, MA) equipped with a quaternary or binary pump system, a column compartment, an autosampler and a UV detector or/and a fluorescence (FLR) detector. For UV detectors, the detection wavelength is set at 280 nm. For fluorescence (FLR) detectors, the detection is set with excitation wavelength at 280 nm and emission wavelength at 352 nm.

The following two sets of HPLC parameters were used, dependent upon the particular polysaccharide being quantified, denoted as “HPLC Conditions A” or “HPLC Conditions B:”

HPLC Conditions A

| | |
|---|---|
| Separation mode | Size-exclusion (SEC) |
| Mobile phase (MP) | 10 mM BisTris, 300 mM NaCl, pH 7 |
| | (or 10 mM BisTris, 150 mM NaCl, pH 7) |
| Gradient | Isocratic |
| Flow Rate | 1.0 mL/min |
| Run Time | 20-25 minutes |
| Column | Shodex PROTEIN KW-803 (8.0 × |
| | 300 mm) |
| Column Temp (° C.) | 35 ± 5 |
| Sample Tray Temp (° C.) | 2-8 |
| UV Detection wavelength | 280 |
| (nm) | |
| FLR detection | Ex. 280 nm; Em. 352 nm |

HPLC Conditions B

| | |
|---|---|
| Separation mode | Size-exclusion (SEC) |
| Mobile phase (MP) | 10 mM BisTris, 150 mM NaCl, pH 7 |
| | (or 10 mM BisTris, 300 mM NaCl, pH 7) |
| Gradient | Isocratic |
| Flow Rate | 0.8 mL/min |
| Run Time | 20-25 minutes |
| Column | TOSOH TSKgel PW × L column, 7.8 × |
| | 300 mm |
| Column Temp (° C.) | 35 ± 5 |
| Sample Tray Temp (° C.) | 2-8 |
| UV Detection wavelength | 280 |
| (nm) | |
| FLR detection | Ex. 280 nm; Em. 352 nm |

### Chromatographic Conditions for SEC Analysis

SEC analysis is run on HPLC using an isocratic mobile phase at a flow rate from 0.4 mL/min to 1.0 mL/min. A typical mobile phase is a neutral or near neutral pH salt buffer with varied salt concentrations. Exemplary mobile phases are disclosed in the specification above. SEC columns useful in the present methods can be obtained commercially, with exemplary columns disclosed in the specification above. The column temperature is typically set at a temperature ranging from 20° C. to 40° C.; the autosampler is typically set at a temperature from 4° C. to 10° C.; and the separation run time is typically from 10 to 30 minutes.

### Preparation of Buffer Used in Antibody-Polysaccharide Binding Reactions

Unless otherwise indicated, the binding reaction buffer used is prepared from the vaccine drug product buffer, wherein the vaccine drug product buffer is as follows:
10 or 20 mM histidine
150 mM NaCl
Polysorbate-20, 0.1-0.2% (w/v)
pH 5.6-6.0.

The binding buffer is prepared by mixing one volume of 100 mM Tris, 600 mM NaCl, pH 9.0 buffer with four volumes of the vaccine drug product buffer to arrive at a final binding buffer solution.

In certain embodiments, the binding buffer is a commercially available PBS buffer. In another embodiment, the binding buffer is the HPLC mobile phase used for SEC analysis, for example 10 mM Bis-Tris, 150 mM NaCl, pH 6.5-7.5 buffer.

### Example 1

### Preparation of Vaccine Polysaccharide Standard Samples for Antibody Binding

### PCV15

Single-serotype polysaccharide standard solutions in unconjugated form (free polysaccharide) for each of the 15 serotypes present in a PCV15 vaccine were prepared using the methodology described in International Publication No. WO 2018/144439 and ranged in concentration from 7 to 16 mg/mL.

For example, 210.4 μL of a serotype 4 (ST-4) polysaccharide standard solution (14.26 mg/mL) was added into 2789.6 μL of HPLC grade water for a 14.26-fold dilution. The resulting solution was then mixed to provide a solution having a polysaccharide concentration of 1.00 mg/mL. 1.00 mL of this resulting solution was then diluted 100-fold with 99.0 mL of HPLC grade water to a provide a stock solution having a polysaccharide concentration of 10.0 μg/mL, which was then divided into 15 equal volume aliquots and stored at −70° C. Prior to analysis, each aliquot was thawed and diluted 10-fold with HPLC grade water to provide final samples for antibody binding (each final sample having a polysaccharide concentration of 1.00 μg/mL).

Final samples of the other 14 serotypes of a PCV 15 vaccine (ST-1, ST-3, ST-5, ST-6A, ST-6B, ST-7F, ST-9V, ST-14, ST-18C, ST-19A, ST-19F, ST-22F, ST-23F, ST-33F) were prepared using the same methodology.

### PCV21

Single-serotype polysaccharide standard solutions in unconjugated form (free polysaccharide) for each of the 21 serotypes present in a PCV21 vaccine were prepared using the methodology described in International Publication No. WO 2019/139692 and ranged in concentration from 7 to 16 mg/mL.

For example, 449.4 μL of a serotype 8 (ST-8) polysaccharide standard solution (0.445 mg/mL) was added into 19550.6 μL of HPLC grade water for a 44.5-fold dilution. The resulting solution was then mixed to a provide a stock solution having a polysaccharide concentration of 10.0 μg/mL, which was then divided into equal volume aliquots and stored at −70° C. Prior to analysis, each aliquot was thawed and diluted 10-fold with HPLC grade water to provide final samples for antibody binding (each final sample having a polysaccharide concentration of 1.00 μg/mL).

Final samples of the other 20 serotypes of the PCV21 vaccine (ST-3, ST-6A, ST-7F, ST-9N, ST-10A, ST-11A, ST-12F, ST-15A, ST-deOAc15B, ST-16F, ST-17F, ST-19A, ST-20A, ST-22F, ST23A, ST-23B, ST-24F, ST-31, ST-33F and ST-35B) were prepared using the same methodology.

### Example 2

### Preparation of Serotype-Specific Knockout Standards

### PCV15

To demonstrate that each anti-serotype antibody binds specifically to its corresponding polysaccharide serotype, serotype-specific knockout standards were prepared.

For a serotype-specific knockout standard from fifteen pneumococcal serotypes, each knockout standard contains fourteen of the following fifteen serotypes with one specific serotype in absence: ST-1, ST-3, ST-4, ST-5, ST-6A, ST-6B, ST-7F, ST-9V, ST-14, ST-18C, ST-19A, ST-19F, ST-22F, ST-23F, ST-33F.

For each of the fifteen serotypes, a 31 μg/mL stock polysaccharide standard solution was prepared from dilution with HPLC grade water. 50 μL of each 31 μg/mL stock polysaccharide standard solution from the following 14 serotypes (without ST-4): ST-1, ST-3, ST-5, ST-6A, ST-6B, ST-7F, ST-9V, ST-14, ST-18C, ST-19A, ST-19F, ST-22F, ST-23F, ST-33F. were added together in a 2 mL microcentrifuge tube was diluted to total volume of 1550 μL with water. The solution was mixed well, which resulted in a 1 μg/mL solution for each of the 14 serotypes (31-fold dilution for each type), ST-4 polysaccharide is excluded (knockout) in this solution.

Individual knockout standard samples for each of the other 14 serotypes were prepared as described above, excluding the specific target serotype.

### PCV21

To demonstrate that each anti-serotype antibody binds specifically to its corresponding polysaccharide serotype, serotype-specific knockout standards were prepared.

For a serotype-specific knockout standard from thirty pneumococcal serotypes (those present in PCV15 and PCV21), each knockout standard contains twenty-nine of the following thirty serotypes with one specific serotype in absence: ST-1, ST-3, ST-4, ST-5, ST-6A, ST-6B, ST-7F, ST-8, ST-9N, ST-9V, ST-10A, ST-11A, ST-12F, ST-14, ST-15A, ST-deOAc15B, ST-16F, ST-17F, ST-18C, ST-19A, ST-19F, ST-20A, ST-22F, ST23A, ST-23B, ST-23F, ST-24F, ST-31, ST-33F and ST-35B.

For each of the thirty serotypes, a 31 μg/mL stock polysaccharide standard solution was prepared from dilution with HPLC grade water.

50 μL of each 31 μg/mL stock polysaccharide standard solution from the following 29 serotypes (without ST-8): ST-1, ST-3, ST-4, ST-5, ST-6A, ST-6B, ST-7F, ST-9N, ST-9V, ST-10A, ST-11A, ST-12F, ST-14, ST-15A, ST-deOAc15B, ST-16F, ST-17F, ST-18C, ST-19A, ST-19F, ST-20A, ST-22F, ST23A, ST-23B, ST-23F, ST-24F, ST-31, ST-33F and ST-35B, were added together in a 2 mL microcentrifuge tube and diluted to total volume of 1550 μL with water. The solution was mixed well, which resulted in a 1 g/mL solution for each of the 29 serotypes (31-fold dilution for each type), ST-8 polysaccharide is excluded (knockout) in this solution.

Individual knockout standard samples for each of the other 29 serotypes were prepared as described above, excluding the specific target serotype.

### Example 3

### Determination of Antibody Serotype-Specificity

### PCV15

Specificity of each antibody to its target polysaccharide serotype (selected from the following fifteen pneumococcal polysaccharide serotypes: 1, 3, 4, 5, 6A, 6B, 7F, 9V, 14, 18C, 19A, 19F, 22F, 23F and 33F), was demonstrated using immunoassay ELISA and Simple Western assays. The specificity of an antibody for each serotype was also confirmed by comparing the antibody binding reaction to confirm formation of serotype-specific antibody-polysaccharide complex from the antibody binding reaction to target serotype with its binding reaction to the mixture of the other 14 polysaccharide serotypes in absence of the target polysaccharide (target polysaccharide knockout sample, as described in Example 2), using HPLC. In all cases, except serotype 6B (minor cross-reactivity with serotype 6A), the APC is detected only in an antibody binding reaction between an anti-serotype antibody and its target polysaccharide. No APC can be detected from antibody binding reactions to target polysaccharide knockout samples. This demonstrates complete specificity for the 14 anti-serotype antibodies (1, 3, 4, 5, 6A, 7F, 9V, 14, 18C, 19A, 19F, 22F, 23F and 33F).

### PCV21

Specificity of each antibody to its target polysaccharide serotype (selected from the following thirty pneumococcal polysaccharide serotypes: ST-1, ST-3, ST-4, ST-5, ST-6A, ST-6B, ST-7F, ST-8, ST-9N, ST-9V, ST-10A, ST-11A, ST-12F, ST-14, ST-15A, ST-deOAc15B, ST-16F, ST-17F, ST-18C, ST-19A, ST-19F, ST-20A, ST-22F, ST23A, ST-23B, ST-23F, ST-24F, ST-31, ST-33F and ST-35B), was demonstrated using immunoassay ELISA and Simple Western assays. The specificity of an antibody for each serotype was also evaluated by comparing the antibody binding reaction to confirm formation of serotype-specific antibody-polysaccharide complex from the antibody binding reaction to target serotype with its binding reaction to the mixture of the other 29 polysaccharide serotypes in absence of the target polysaccharide (target polysaccharide knockout sample, as described in Example 2), using HPLC. In all cases, except for serotype 6B (minor cross-reactivity with serotype 6A), the APC is detected only in an antibody binding reaction between an anti-serotype antibody and its target polysaccharide. No APC can be detected from antibody binding reactions to target polysaccharide knockout samples. This demonstrates specificity for the 29 anti-serotype antibodies (ST-1, ST-3, ST-4, ST-5, ST-6A, ST-7F, ST-8, ST-9N, ST-9V, ST-10A, ST-11A, ST-12F, ST-14, ST-15A, ST-deOAc15B, ST-16F, ST-17F, ST-18C, ST-19A, ST-19F, ST-20A, ST-22F, ST23A, ST-23B, ST-23F, ST-24F, ST-31, ST-33F and ST-35B).

### Example 4

### Preparation of a Vaccine Sample Stock Solution

### PCV15

1.5 to 3.0 mL of a PCV15 vaccine drug product (containing adjuvant and 4 mcg/mL of each of the following polysaccharide serotypes: 1, 3, 4, 5, 6A, 6B, 7F, 9V, 14, 18C, 19A, 19F, 22F, 23F and 33F) was used to prepare a vaccine drug product sample(s), which was then put into a microcentrifuge tube and centrifuged at 5 to 15 kg for 20 minutes. The supernatant was collected, then diluted with one quarter volume of a suitable buffer, and the resulting mixture (the “vaccine sample stock solution”) was then used directly or further purified prior to being reacted with a serotype-specific antibody. To the resulting centrifugated vaccine drug product sample was added an excess of an antibody that is specific for the vaccine carrier protein in order to capture carrier protein, and the resulting mixture was incubated at room temperature for a period of about 8 hours. The solution was then quenched using Protein A/G beads, and the quenched solution was filtered to remove beads. The filtrate was then incubated at room temperature for a time of about 5 hours. These steps may be repeated, if necessary, to ensure that all conjugated polysaccharides are captured, and only unconjugated (free) polysaccharides are present, thereby providing a vaccine sample for analysis (a “vaccine stock sample solution”). A dilution factor is then calculated based on the starting sample volume and the sample volume after sample preparation.

### PCV21

1.5 to 3.0 mL of a 21-valent vaccine (PCV21) drug product (containing 8 mcg/mL of each of the following polysaccharide serotypes: ST-3, ST-6A, ST-7F, ST-8, ST-9N, ST-10A, ST-11A, ST-12F, ST-15A, ST-deOAc15B, ST-16F, ST-17F, ST-19A, ST-20A, ST-22F, ST23A, ST-23B, ST-24F, ST-31, ST-33F and ST-35B) was used to prepare a vaccine drug product sample(s), which was then put into a microcentrifuge tube and centrifuged at 5 to 15 kg for 20 minutes. The supernatant was collected, then diluted with one quarter volume of a suitable buffer, and the resulting mixture (the “vaccine sample stock solution”) was then used directly or further purified prior to being reacted with a serotype-specific antibody. To the resulting centrifugated vaccine drug product sample was added an excess of an antibody that is specific for the vaccine carrier protein in order to capture carrier protein, and the resulting mixture was incubated at room temperature for a period of about 8 hours. The solution was then quenched using Protein A/G beads, and the quenched solution was filtered to remove beads. The filtrate was then incubated at room temperature for a time of about 5 hours. These steps may be repeated, if necessary, to ensure that all conjugated polysaccharides are captured, and only unconjugated (free) polysaccharides are present, thereby providing a vaccine sample for analysis (a “vaccine stock sample solution”). A dilution factor is then calculated based on the starting sample volume and the sample volume after sample preparation.

### Example 5

### General Method for Generation of Polysaccharide/Anti-Polysaccharide Antibody Complex and Generation of Assay Standard Curve

### Step A—Preparation of Polysaccharide Anti-Polysaccharide Antibody Complex

A polysaccharide/anti-polysaccharide antibody complex standard curve can be made by mixing a polysaccharide serotype standard with an excess of corresponding anti-polysaccharide antibody at one or more different polysaccharide concentrations. The resulting binding reaction mixture is then incubated at a temperature from 20-40° C. for 0.5 to 5 hours to provide an “antibody-polysaccharide complex.”

### Step B—Generation of Standard Curve

A standard curve can be generated using chromatography peak areas from the antibody-polysaccharide complex (prepared in Step A) vs the polysaccharide concentrations ([Ps]) in each of the binding reactions. The intercept and slope of this standard curve are used to calculate the polysaccharide concentration in a vaccine drug product sample, as described below herein.

### Example 6

### General Method for Generation of Vaccine Antibody-Polysaccharide Complex and HPLC Analysis

A vaccine sample stock solution is separated into a specific number of aliquots, equal to the number of different polysaccharide serotypes contained in the vaccine drug product. Each aliquot is put in an HPLC vial, and to each separate aliquot is added one antibody specific to a single polysaccharide that is present in the vaccine drug product, such that separate binding reactions are performed for each serotype present, and each of the vaccine stock sample solution aliquots contains a serotype-specific antibody that is specific for a different one of the individual polysaccharides present in the vaccine drug product.

For each serotype, the polysaccharide concentration in the vaccine stock sample solution is calculated from standard curve intercept and slope using Equation-1:

$$[ Vaccine Ps in binding reaction ] = \frac{Vaccine sample peak area - STD intercept}{STD slope} \qquad Equation - 1$$

The polysaccharide concentration of the vaccine drug product will be calculated using dilution factor times the polysaccharide concentration in the vaccine stock sample solution binding reaction (Equation-2):

$$[ Vaccine product polysaccharide ] = Dilution * [ Vaccine Ps in binding reaction ] \qquad Equation - 2$$

The dilution factor in Equation-2 is equal to dilution in vaccine sample binding reaction times the sample preparation dilution factor. (Example can be seen in Example 4 and/or Example 7.

The standard curve can also be generated using standard polysaccharide, the following equations (Equation-1a and 1b) will be used for calculation of polysaccharide concentration in the reaction. The polysaccharide concentration in the reaction will be converted to vaccine drug product polysaccharide concentration through Equation-2.

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

### Example 7

### Preparation of PCV15 Vaccine Drug Product Sample

### PCV15

### Step A—Removal of the Vaccine Adjuvant and Adjuvant Bound Species

A PCV15 vaccine drug product (containing adjuvant and 4 mcg/mL of each of the following polysaccharide serotypes: 1, 3, 4, 5, 6A, 6B, 7F, 9V, 14, 18C, 19A, 19F, 22F, 23F and 33F) was taken from 3 product vials (each vial containing 0.5 mL of vaccine product) and was combined, then split into two 2 mL microcentrifuge tubes. Each tube was centrifuged at 10,000 rpm for 5 minutes. The supernatant was combined (total volume of 1.0 mL), and then the following were added: 0.25 mL of 100 mM Tris, 600 mM NaCl, and pH 9.0 buffer. The resulting mixture was used in the next step.

### Step B—Removal of CRM197 Protein Carrier

71 μg of anti-CRM197 antibodies in solution were added to the product of step A in a 2 mL microcentrifuge tube containing dry Protein A/G magnetic beads. The solution was mixed well and allowed to incubate at room temperature for about 4 hours. The magnetic beads were then separated from the supernatant solution (using n a DynaMag™-2 Magnet), to provide a vaccine drug product sample that is free of all CRM197 species.

### PCV21

### Step A—Addition of Vaccine Adjuvant to Remove Adjuvant Bound Species

A 21-valent vaccine drug product (containing 8 mcg/mL of each of the following polysaccharide serotypes: ST-3, ST-6A, ST-7F, ST-8, ST-9N, ST-10A, ST-11A, ST-12F, ST-15A, ST-deOAc15B, ST-16F, ST-17F, ST-19A, ST-20A, ST-22F, ST23A, ST-23B, ST-24F, ST-31, ST-33F and ST-35B) was taken from 2 product vials (each vial containing 0.5 mL of vaccine product) and was combined. An equal volume of 2× vaccine adjuvant was then added to the vaccine product and allowed to incubate for at least 16 hours while rotating. After rotation, the solution was vortexed and then centrifuged at 10,000 rpm for 5 minutes. The supernatant was collected and then the following was added: 0.25 mL of 100 mM Tris, 600 mM NaCl, pH 9.0 buffer. The resulting mixture was used in the next step.

### Step B—Removal of CRM197 Protein Carrier

120 μg of anti-CRM197 antibodies in solution were added to the product of step A in a microcentrifuge tube containing dry Protein A/G magnetic beads. The solution was mixed well and allowed to incubate at room temperature for about 1 hour. The magnetic beads were then separated from the supernatant solution (using a DynaMag™-2 Magnet). An additional 120 μg of anti-CRM197 antibodies in solution were added to the product in a microcentrifuge tube containing fresh dry Protein A/G magnetic beads. The solution was mixed well and allowed to incubate at room temperature for at least 16 hours. The magnetic beads were then separated from the supernatant solution (using a DynaMag™-2 Magnet), to provide a vaccine drug product sample that is free of all CRM197 species.

### Example 8

### Serotype-Specific Quantitation of ST-33F Free Polysaccharide in Multi-Valent Vaccine Drug Product

### Step A—ST-33F Polysaccharide Standard Anti ST-33F Antibody Binding Reaction

A polysaccharide/antibody binding reaction was carried out by pipetting 5 μL of a ST-33F polysaccharide standard solution (1 μg/mL, standard solution prepared according to the methodology described in Example 1) into 20 μL of anti-ST-33F IgG mAb solution (0.15 mg/mL, solution in binding buffer), and adding additional binding buffer, as indicated in Table 8a below. The resulting binding reaction was then allowed to incubate at room temperature for 1 hour. This reaction was carried out in the same manner six additional times using the same amount of ST-33F IgG mAb solution, and the following amounts of ST-33F polysaccharide standard solution: 8 μL, 10 μL, 15 μL, 20 μL, 25 μL, and 30 μL. Table 8a summarizes the stoichiometry of each of the seven binding reactions.

TABLE 8a

| | 1 μg/mL | 0.15 mg/mL | Binding | Total | |
|---|---|---|---|---|---|
| | ST33F Ps | anti-ST33F | Buffer | Vol | [ST33F] |
| ST33F STD binding | STD (μL) | IgG (μL) | (μL) | (μL) | (μg/mL) |
| ST33F-STD-1 | 5 | 20 | 175 | 200 | 0.025 |
| ST33F-STD-2 | 8 | 20 | 172 | 200 | 0.040 |
| ST33F-STD-3 | 10 | 20 | 170 | 200 | 0.050 |
| ST33F-STD-4 | 15 | 20 | 165 | 200 | 0.075 |
| ST33F-STD-5 | 20 | 20 | 160 | 200 | 0.100 |
| ST33F-STD-6 | 25 | 20 | 155 | 200 | 0.125 |
| ST33F-STD-7 | 30 | 20 | 150 | 200 | 0.150 |

### Step B—Vaccine Anti ST-33F Antibody Binding Reaction

150 μL of a PCV15 vaccine sample stock solution (prepared from a PCV15 vaccine drug product, using the methods described in Example 4) was incubated with 30 μL of anti-ST-33F IgG mAb solution (0.15 mg/mL in binding buffer), and 120 μL of binding buffer at room temperature for two hours. The incubated antibody-vaccine binding reaction mixture was then analyzed using HPLC, as described below in Step D.

### Step C—HPLC Analysis of Polysaccharide Standard Binding Reactions and Preparation of Standard Curve

Each of the seven ST-33F polysaccharide standard binding reactions prepared as described in Step A were individually analyzed using chromatography condition A (described above in the General Assay Methods section, and using an 80 μL injection volume of each binding reaction mixture). The ST-33F serotype for each of the five binding reactions are shown below in the second column of Table 8b. The ST-33F polysaccharide fluorescence peak areas for each corresponding ST-33F concentration are shown in the third column of Table 8b.

TABLE 8b

| ST33F STD | [ST33F] (ug/mL) | FLR peak area |
|---|---|---|
| ST33F-STD-1 | 0.025 | 1912706 |
| ST33F-STD-2 | 0.04 | 2175152 |
| ST33F-STD-3 | 0.05 | 2463619 |
| ST33F-STD-4 | 0.075 | 2642049 |
| ST33F-STD-5 | 0.1 | 3013242 |
| ST33F-STD-6 | 0.125 | 3350148 |
| ST33F-STD-7 | 0.15 | 3871957 |
| RSQ (R²) | 0.9865 | NA |
| Intercept | 1591267 | NA |
| Slope | 14672575 | NA |

A seven-point standard curve was plotted using the ST-33F concentration (in μg/mL) as the x-axis, and the ST-33F/anti-ST-33F antibody APC fluorescence peak area as the y-axis. The slope and intercept of this curve were calculated and are set forth in Table 8b. A calculated R squared (RSQ) value of 0.9865 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition A (as described in the General Methods section above). The antibody/polysaccharide complex fluorescence peak area signal generated (see Table 8c) was then used in Step E.

TABLE 8c

| | | [ST-33F] | Dilution | |
|---|---|---|---|---|
| | mAb-Ps | in binding | in | Dilution in |
| | Complex FLR | reaction | binding | sample |
| Vaccine DP ST33F | peak area | (μg/mL) | reaction | preparation |
| Vaccine DP ST33F | 3075089 | 0.101 | 2 | 1.3 |

### Step E—Quantification of Free ST-33F in PCV15 Vaccine Drug Product

The complex peak area provided in Step D, was used along with the slope and intercept of the polysaccharide standard curve from Step C, to calculate the concentration of ST-33F polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 8c above).

This resulted in a calculated value for the concentration of free ST-33F polysaccharide in the PCV15 vaccine drug product of 0.263 μg/mL, as summarized in Table 8d below.

TABLE 8d

| | | [ST-33F] |
|---|---|---|
| | [ST-33F] | (μg/mL) in |
| | in | PCV15 |
| Vaccine | binding | vaccine |
| DP ST- | reaction | drug |
| 33F | (μg/mL) | product |
| Vaccine | 0.101 | 0.263 |
| DP ST- | | |
| 33F | | |

### Example 9

### Serotype-Specific Quantitation of ST-4 Free Polysaccharide in a Multi-Valent Vaccine Drug Product

### Step A—ST-4 Polysaccharide Standard/Anti ST-4 Antibody Binding Reaction

A polysaccharide/antibody binding reaction was carried out by pipetting 2 μL of a ST-4 polysaccharide standard solution (1 μg/mL, standard solution prepared according to the methodology described in Example 1) into 20 μL of anti-ST-4 IgG mAb solution (0.1 mg/mL, solution in binding buffer), and adding additional binding buffer until a total reaction mixture volume of 200 μL was achieved. The resulting binding reaction was then allowed to incubate at room temperature for 1 hour. This reaction was carried out in the same manner four additional times using the same amount of ST-4 IgG mAb solution, and the following amounts of ST-4 polysaccharide standard solution: 5 μL, 10 μL, 20 μL, and 30 μL (additional binding buffer was added to each separate binding reaction to achieve a total volume of 200 μL for each reaction). Table 9a summarizes the stoichiometry of each of the five binding reactions:

TABLE 9a

| | 1 μg/mL | 0.1 mg/mL | Binding | Total |
|---|---|---|---|---|
| | ST-4 Ps | anti-ST-4 | Buffer | Vol |
| ST-4 STD binding | STD (μL) | IgG (μL) | (μL) | (μL) |
| ST-4-STD-1 | 2 | 20 | 178 | 200 |
| ST-4-STD-2 | 5 | 20 | 175 | 200 |
| ST-4-STD-3 | 10 | 20 | 170 | 200 |
| ST-4-STD-4 | 20 | 20 | 160 | 200 |
| ST-4-STD-5 | 30 | 20 | 150 | 200 |

before being analyzed using HPLC, as described below in Step C.

### Step B—Vaccine/Anti ST-4 Antibody Binding Reaction

100 μL of a PCV15 vaccine sample stock solution (prepared from a PCV15 vaccine drug product, using the methods described in Example 4) was incubated with 20 μL of anti-ST-4 IgG mAb solution (0.1 mg/mL in binding buffer), and 80 μL of binding buffer at room temperature for one hour. The incubated antibody-vaccine binding reaction mixture was then analyzed using HPLC, as described below in Step D.

### Step C—HPLC Analysis of Polysaccharide Standard Binding Reactions and Preparation of Standard Curve

Each of the five ST-4 polysaccharide standard binding reactions prepared as described in Step A were individually analyzed using chromatography condition B (described above in the General Assay Methods section, and using a 100 μL injection volume of each binding reaction mixture). The ST-4 serotype for each of the five binding reactions are shown below in the second column of Table 9b. The ST-4 polysaccharide fluorescence peak areas for each corresponding ST-4 concentration are shown in the third column of Table 9b.

TABLE 9b

| ST-4 STD | [ST-4] (μg/mL) | FLR peak area |
|---|---|---|
| ST-4-STD-1 | 0.01 | 40 |
| ST-4-STD-2 | 0.025 | 98 |
| ST-4-STD-3 | 0.05 | 212 |
| ST-4-STD-4 | 0.1 | 429 |
| ST-4-STD-5 | 0.15 | 655 |
| RSQ | 0.9998 | NA |
| Intercept | −8.454066265 | NA |
| Slope | 4406.777108 | NA |

A five-point standard curve was plotted using the ST-4 concentration (in ug/mL) as the x-axis, and the ST-4/anti-ST-4 antibody APC fluorescence peak area as the y-axis. The slope and intercept of this curve were calculated and are set forth in Table 9b. A calculated R squared (RSQ) value of 0.9998 indicates good linearity.

### Step D—HPLC Analysis of Vaccine Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 9c) was then used in Step E.

TABLE 9c

| | | | | | [ST-4] |
|---|---|---|---|---|---|
| | mAb-Ps | [ST-4] in | Dilution | | (μg/mL) |
| | Complex | binding | in | Dilution in | in PCV15 |
| Vaccine | FLR | reaction | binding | sample | vaccine |
| DP ST-4 | peak area | (μg/mL) | reaction | preparation | drug product |
| Vaccine | 203 | 0.0480 | 2 | 1.3 | 0.12 |
| DP ST-4 | | | | | |

### Step E—Quantification of Free ST-4 in PCV15 Vaccine Drug Product

The complex peak area provided in Step D, was used along with the slope and intercept of the polysaccharide standard curve from Step C, to calculate the concentration of ST-4 polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the sample preparation dilution factor in Step A (see dilution values in Table 9c above).

This resulted in a calculated value for the concentration of free ST-4 polysaccharide in the PCV15 vaccine drug product of 0.12 μg/mL, as summarized in Table 9d below.

TABLE 9d

| | [ST-4] in | [ST-4] (μg/mL) in |
|---|---|---|
| | binding reaction | PCV15 vaccine |
| Vaccine DP ST-4 | (μg/mL) | drug product |
| Vaccine DP ST-4 | 0.0480 | 0.12 |

### Example 10

### Serotype-Specific Quantitation of ST-1 Free Polysaccharide in a Multi-Valent Vaccine Drug Product

### Step A—ST-1 Polysaccharide Standard/Anti ST-1 Antibody Binding Reaction

A polysaccharide/anti-polysaccharide antibody binding reaction was carried out by pipetting 1 μL of a ST-1 polysaccharide standard solution (1 μg/mL, standard solution prepared according to the methodology described in Example 1) into 20 μL of anti-ST-1 IgG mAb solution (0.1 mg/mL, solution in binding buffer), and adding additional binding buffer until a total reaction mixture volume of 200 μL was achieved. The resulting binding reaction was then allowed to incubate at room temperature for 1 hour. This reaction was carried out in the same manner four additional times using the same amount of ST-1 IgG mAb solution, and the following amounts of ST-1 polysaccharide standard solution: 3 μL, 5 μL, 8 μL, and 10 μL (additional binding buffer was added to each separate binding reaction to achieve a total volume of 200 μL for each reaction). Table 10a summarizes the stoichiometry of each of the five binding reactions.

TABLE 10a

| | 1.0 μg/mL | 0.1 mg/mL | Binding | Total | Total Ps |
|---|---|---|---|---|---|
| ST1 STD | ST1 Ps | anti ST1 | Buffer | Vol | Amt |
| binding | STD (μL) | mAb (μL) | (μL) | (μL) | (μg) |
| ST1 STD-1 | 1 | 20 | 179 | 200 | 0.001 |
| ST1 STD-2 | 3 | 20 | 177 | 200 | 0.003 |
| ST1 STD-3 | 5 | 20 | 175 | 200 | 0.005 |
| ST1 STD-4 | 8 | 20 | 172 | 200 | 0.008 |
| ST1 STD-5 | 10 | 20 | 170 | 200 | 0.010 |

### Step B—Vaccine/Anti ST-1 Antibody Binding Reaction

20 μL of a PCV15 vaccine sample stock solution (prepared from a PCV15 vaccine drug product, using the method described in Example 4) was incubated with 20 μL of anti-ST-1 IgG mAb solution (0.1 mg/mL in binding buffer), and 160 μL of binding buffer at room temperature for one hour. The incubated binding reaction mixture was then analyzed using HPLC, as described below in Step D.

### Step C—HPLC Analysis of Polysaccharide Standard Binding Reactions and Preparation of Standard Curve

Each of the five ST-1 polysaccharide standard binding reactions prepared as described in Step A (and referred to as ST-1 STD-1 through ST-1 STD-5) were individually analyzed using chromatography condition A (described above in the General Assay Methods section), with an 80 μL injection volume of each binding reaction mixture. Each HPLC analysis was done in duplicate, and the data for each of the five polysaccharide standard binding reactions, are presented in Table 10b.

TABLE 10b

| ST-1 binding | 1.0 μg/mL ST1 | ST1 Amt per | Injection-1 | Injection-2 | Average ST-1 |
|---|---|---|---|---|---|
| reaction | Ps STD (μL) | INJ (μg) | Peak area | Peak area | FLR peak area |
| ST1 STD-1 | 1 | 0.0004 | 408554 | 437960 | 423257 |
| ST1 STD-2 | 3 | 0.0012 | 1183930 | 1215585 | 1199757 |
| ST1 STD-3 | 5 | 0.002 | 2306829 | 2386428 | 2346628 |
| ST1 STD-4 | 8 | 0.0032 | 4246736 | 4252042 | 4249389 |
| ST1 STD-5 | 10 | 0.004 | 5232747 | 5262548 | 5247647 |

| | | |
|---|---|---|
| RSQ | 0.9948 | NA |
| Intercept | −301755.28 | NA |
| Slope | 1386616057 | NA |

A five-point standard curve was plotted using the ST-1 amount per injection (μg) as the x-axis, and the average ST-1/anti-ST-1 antibody APC fluorescence peak area as the y-axis. The slope and intercept of this curve were calculated and are provided in Table 10b. The calculated R squared (RSQ) value of 0.9948 indicates good linearity for the curve.

### Step D—HPLC Analysis of Vaccine/Antibody Binding Reactions

80 μL samples of the vaccine/antibody binding reaction mixture prepared in Step B were analyzed in duplicate by HPLC using Chromatographic condition A (as described in the General Assay Methods section above). The antibody/polysaccharide complex fluorescence peak area signals were averaged, and presented in Table 10c, along with the amount of ST-1 polysaccharide serotype present in each HPLC injection sample. These data were used as described in Step E to calculate the ST-1 polysaccharide serotype concentration in the PCV15 vaccine drug product.

TABLE 10c

| Vaccine | Dilution from | Dilution from | Inj Volume | | | Average | ST-1 PS per |
|---|---|---|---|---|---|---|---|
| DP ST-1 | DP Prep | binding Rx | (μL) | FLR-1 | FLR-2 | FLR | INJ (μg) |
| ST-1 DP | 1.3 | 10 | 80 | 2429659 | 2479210 | 2454434 | 0.002 |

### Step E—Quantification of Free ST-4 in PCV15 Vaccine Drug Product

The average complex peak area provided in Step D, was used along with the slope and intercept of the polysaccharide standard curve from Step C, to calculate the concentration of ST-1 polysaccharide in the vaccine/antibody binding reaction of Step B, using Equations 1a and 1b:

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

This resulted in a calculated value of 0.025 μg/mL for the concentration of free ST-1 polysaccharide in the binding reaction of step D, and a in a calculated value of 0.32 μg/mL for the concentration of free ST-1 polysaccharide in the PCV15 vaccine drug product, as summarized in Table 10d.

TABLE 10d

| | ST-1 [Ps] in | Vaccine DP |
|---|---|---|
| Vaccine DP ST-1 | reaction (μg/mL) | [ST1] (μg/mL) |
| ST-1 DP | 0.025 | 0.32 |

### Example 11

### Serotype-Specific Quantitation of ST-6B Free Polysaccharide in Multi-Valent Vaccine Drug Product

### Step A—ST-6B Polysaccharide Standard Anti ST-6B Antibody Binding Reaction

A polysaccharide/anti-polysaccharide antibody binding reaction was carried out by pipetting 1 μL of a ST-6B polysaccharide standard solution (1 μg/mL, standard solution prepared according to the methodology described in Example 1) into 20 μL of anti-ST-6B IgG mAb solution (0.1 mg/mL, solution in binding buffer), and adding additional binding buffer until a total reaction mixture volume of 200 μL was achieved. The resulting binding reaction was then allowed to incubate at room temperature for 2 hours. This reaction was carried out in the same manner four additional times using the same amount of ST-6B IgG mAb solution, and the following amounts of ST-6B polysaccharide standard solution: 3 μL, 5 μL, 8 μL, and 10 μL (additional binding buffer was added to each separate binding reaction to achieve a total volume of 200 μL for each reaction). Table 11a summarizes the stoichiometry of each of the five binding reactions.

TABLE 11a

| | 1.0 μg/mL | 0.1 mg/mL | Binding | Total | |
|---|---|---|---|---|---|
| 6B STD | 6B Ps | anti- 6B | Buffer | Vol | Ps Amt |
| Binding | STD (μL) | mAb (μL) | (μL) | (μL) | (μg) |
| ST6B STD-1 | 1 | 20 | 179 | 200 | 0.001 |
| ST6B STD-2 | 3 | 20 | 177 | 200 | 0.003 |
| ST6B STD-3 | 5 | 20 | 175 | 200 | 0.005 |
| ST6B STD-4 | 8 | 20 | 172 | 200 | 0.008 |
| ST6B STD-5 | 10 | 20 | 170 | 200 | 0.010 |

### Step B—Vaccine Anti ST-6B Antibody Binding Reaction

20 μL of a PCV15 vaccine sample stock solution (prepared from a PCV15 vaccine drug product, using the method described in Example 4) was incubated with 20 μL of anti-ST-6B IgG mAb solution (0.1 mg/mL in binding buffer), and 160 μL of binding buffer at room temperature for two hours. The incubated binding reaction mixture was then analyzed using HPLC, as described below in Step D.

### Step C—HPLC Analysis of Polysaccharide Standard Binding Reactions and Preparation of Standard Curve

Each of the five ST-6B polysaccharide standard binding reactions prepared as described in Step A (and referred to as ST-6B STD-1 through ST-6B STD-5) were individually analyzed using chromatography condition A (described above in the General Assay Methods section), with an 80 μL injection volume of each binding reaction mixture. Each HPLC analysis was done in duplicate, and the data for each of the five polysaccharide standard binding reactions are presented in Table 11b.

TABLE 11b

| | | | Peak area | | |
|---|---|---|---|---|---|
| ST6B binding | | ST6B Amt per | | | Avg peak |
| Reaction | Inj Vol (μL) | INJ (μg) | INJ-1 | INJ-2 | area |
| ST6B STD-1 | 80 | 0.0004 | 237424 | 253908 | 245666 |
| ST6B STD-2 | 80 | 0.0012 | 854997 | 848657 | 851827 |
| ST6B STD-3 | 80 | 0.002 | 1401572 | 1429440 | 1415506 |
| ST6B STD-4 | 80 | 0.0032 | 2291100 | 2323182 | 2307141 |
| ST6B STD-5 | 80 | 0.004 | 2836142 | 2867947 | 2852044 |

| | | |
|---|---|---|
| RSQ | 0.9998 | NA |
| Intercept | 31406.545 | NA |
| Slope | 724927336 | NA |

A five-point standard curve was plotted using the ST-6B amount per injection (ug) as the x-axis, and the average ST-6B/anti-ST-6B antibody APC fluorescence peak area as the y-axis. The slope and intercept of this curve were calculated and are provided in Table 11b. The calculated R squared (RSQ) value of 0.9998 indicates good linearity for the curve.

### Step D—HPLC Analysis of Vaccine/Antibody Binding Reactions

80 μL samples of the vaccine/antibody binding reaction mixture prepared in Step B were analyzed in duplicate by HPLC using Chromatographic condition A (as described in the General Assay Methods section above). The antibody/polysaccharide complex fluorescence peak area signals were averaged, and presented in Table 11c, along with the amount of ST-6B polysaccharide serotype present in each HPLC injection sample. These data were used as described in Step E to calculate the ST-6B polysaccharide serotype concentration in the PCV15 vaccine drug product.

TABLE 11c

| Vaccine | Dilution from | Dilution from | Inj Volume | | | Average | ST-6B PS per |
|---|---|---|---|---|---|---|---|
| DP ST-6B | DP Prep | binding Rx | (μL) | FLR-1 | FLR-2 | FLR | INJ (μg) |
| ST-6B DP | 1.3 | 10 | 80 | 1479167 | 1468792 | 1473979 | 0.0021 |

### Step E—Quantification of Free ST-6B in PCV15 Vaccine Drug Product

The average complex peak area provided in Step D, was used along with the slope and intercept of the polysaccharide standard curve from Step C, to calculate the concentration of ST-6B polysaccharide in the vaccine/antibody binding reaction of Step B, using Equations 1a and 1b:

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

This resulted in a calculated value of 0.026 μg/mL for the concentration of free ST-6B polysaccharide in the binding reaction of step D, and a calculated value of 0.34 μg/mL for the concentration of free ST-6B polysaccharide in the PCV15 vaccine drug product, as summarized in Table 11d.

TABLE 11d

| | [ST-6B] in | Vaccine DP |
|---|---|---|
| Vaccine DP ST-6B | reaction (μg/mL) | [ST-6B] (μg/mL) |
| ST-6B DP | 0.026 | 0.34 |

### Example 12

### Serotype-Specific Quantitation of ST-3 Free Polysaccharide in Multi-Valent Vaccine Drug Product

### Step A—ST-3 Polysaccharide Standard Anti ST-3 Antibody Binding Reaction

A polysaccharide/antibody binding reaction was carried out by pipetting 2 μL of a ST-3 polysaccharide standard solution (I ug/mL, standard solution prepared according to the methodology described in Example 1) into 20 μL of anti-ST-3 IgG mAb solution (0.1 mg/mL, solution in binding buffer), and adding additional binding buffer until a total reaction mixture volume of 200 μL was achieved. The resulting binding reaction was then allowed to incubate at room temperature for 1 hour. This reaction was carried out in the same manner four additional times using the same amount of ST-3 IgG mAb solution, and the following amounts of ST-3 polysaccharide standard solution: 5 μL, 10 μL, and 20 μL (additional binding buffer was added to each separate binding reaction to achieve a total volume of 200 μL for each reaction). Table 12a summarizes the stoichiometry of each of the five binding reactions:

TABLE 12a

| | 1 μg/mL | 0.1 mg/mL | Binding | Total | Total Ps |
|---|---|---|---|---|---|
| ST3 STD | ST3 Ps | ST3 mAb | Buffer | Vol | Amt |
| Binding | STD (μL) | (μL) | (μL) | (μL) | (μg) |
| ST3 STD-1 | 2 | 20 | 178 | 200 | 0.002 |
| ST3 STD-2 | 5 | 20 | 175 | 200 | 0.005 |
| ST3 STD-3 | 10 | 20 | 170 | 200 | 0.010 |
| ST3 STD-4 | 20 | 20 | 160 | 200 | 0.020 |

### Step B—Vaccine Anti ST-3 Antibody Binding Reaction

0.5 mL of a PCV15 vaccine sample stock solution (prepared from a PCV15 vaccine drug product, using the method described in Example 4) was desalted into PBS using a 2 mL Pierce desalting column. 75 μL of desalted vaccine sample was bound to 30 μL of anti-ST-3 IgG mAb solution (0.15 mg/mL in binding buffer) in 195 μL of binding buffer, then the mixture was incubated at room temperature for one hour. The incubated binding reaction mixture was then analyzed using HPLC, as described below in Step D.

### Step C—HPLC Analysis of Polysaccharide Standard Binding Reactions and Preparation of Standard Curve

Each of the four ST-3 polysaccharide standard binding reactions prepared as described in Step A (and referred to as ST-3 STD-1 through ST-3 STD-4) were individually analyzed using chromatography condition B (described above in the General Assay Methods section), with an 80 μL injection volume of each binding reaction mixture. Each HPLC analysis was done in duplicate, and the data for each of the five polysaccharide standard binding reactions, are presented in Table 12b.

TABLE 12b

| | Injection | Ps Amt per | |
|---|---|---|---|
| ST-3 STD | Volume (μL) | INJ (μg) | FLR |
| ST-3 STD 1 | 80 | 0.0008 | 492802 |
| ST-3 STD 2 | 80 | 0.002 | 1267278 |
| ST-3 STD 3 | 80 | 0.004 | 2604852 |
| ST-3 STD 4 | 80 | 0.008 | 5076903 |

| | | |
|---|---|---|
| RSQ | 0.9997 | NA |
| Intercept | 4168 | NA |
| Slope | 636835345 | NA |

A five-point standard curve was plotted using the ST-3 amount per injection (ug) as the x-axis, and the average ST-3/anti-ST-3 antibody APC fluorescence peak area as the y-axis. The slope and intercept of this curve were calculated and are provided in Table 12b. The calculated R squared (RSQ) value of 0.9997 indicates good linearity for the curve.

### Step D—HPLC Analysis of Vaccine/Antibody Binding Reactions

80 μL samples of the vaccine/antibody binding reaction mixture prepared in Step B were analyzed in duplicate by HPLC using Chromatographic condition B (as described in the General Assay Methods section above). The antibody/polysaccharide complex fluorescence peak area signals were averaged, and presented in Table 12c, along with the amount of ST-3 polysaccharide serotype present in each HPLC injection sample. These data were used as described in Step E to calculate the ST-3 polysaccharide serotype concentration in the PCV15 vaccine drug product.

TABLE 12c

| | | | | | | | | [ST3] | Vaccine |
|---|---|---|---|---|---|---|---|---|---|
| ST-3 | | | | | | | Ps Amt | in Rx | DP |
| vaccine | DP Prep | Rx Prep | Inj Vol | | | Average | per INJ | solution | [ST3] |
| DP | Dilution | Dilution | (μL) | FLR-1 | FLR-2 | FLR | (μg) | (μg/mL) | (μg/mL) |
| ST-3 DP | 1.3 | 4 | 80 | 4293420 | 4403190 | 4348305 | 0.0068 | 0.085 | 0.44 |

### Step E—Quantification of Free ST-3 in PCV15 Vaccine Drug Product

The average complex peak area provided in Step D, was used along with the slope and intercept of the polysaccharide standard curve from Step C, to calculate the concentration of ST-3 polysaccharide in the vaccine/antibody binding reaction of Step B, using Equations 1a and 1b:

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

This resulted in a calculated value of 0.025 μg/mL for the concentration of free ST-3 polysaccharide in the binding reaction of step D, and a calculated value of 0.32 μg/mL for the concentration of free ST-3 polysaccharide in the PCV15 vaccine drug product, as summarized in Table 12d.

TABLE 12d

| | [ST-3] in Rx | Vaccine DP |
|---|---|---|
| ST-3 vaccine DP | solution (μg/mL) | [ST-3] (μg/mL) |
| ST-3 DP | 0.025 | 0.32 |

### Example 13

### Serotype-Specific Quantitation of ST-5 Free Polysaccharide in Multi-Valent Vaccine Drug Product

### Step A—ST-5 Polysaccharide Standard Anti ST-5 Antibody Binding Reaction

A polysaccharide/anti-polysaccharide antibody binding reaction was carried out by pipetting 2 μL of a ST-5 polysaccharide standard solution (1 μg/mL, standard solution prepared according to the methodology described in Example 1) into 20 μL of anti-ST-5 IgG mAb solution (0.1 mg/mL, solution in binding buffer), and adding additional binding buffer until a total reaction mixture volume of 200 μL was achieved. The resulting binding reaction was then allowed to incubate at room temperature for 1 hour. This reaction was carried out in the same manner four additional times using the same amount of ST-5 IgG mAb solution, and the following amounts of ST-5 polysaccharide standard solution: 5 μL, 10 μL, 20 μL, and 30 μL (additional binding buffer was added to each separate binding reaction to achieve a total volume of 200 μL for each reaction). Table 13a summarizes the stoichiometry of each of the five binding reactions.

TABLE 13a

| | 1 μg/mL | 0.1 mg/mL | Binding | Total | Ps Amt |
|---|---|---|---|---|---|
| | ST5 Ps | anti-ST5 | Buffer | Vol | Per INJ |
| ST5 STD binding | STD (μL) | IgG (μL) | (μL) | (μL) | (μg) |
| ST5 STD-1 | 2 | 20 | 178 | 200 | 0.0008 |
| ST5 STD-2 | 5 | 20 | 175 | 200 | 0.002 |
| ST5 STD-3 | 10 | 20 | 170 | 200 | 0.004 |
| ST5 STD-4 | 20 | 20 | 160 | 200 | 0.008 |
| ST5 STD-5 | 30 | 20 | 150 | 200 | 0.012 |

### Step B—Vaccine/Anti ST-5 Antibody Binding Reaction

50 μL of a PCV15 vaccine sample stock solution (prepared from a PCV15 vaccine drug product, using the method described in Example 4) was incubated with 20 μL of anti-ST-5 IgG mAb solution (0.1 mg/mL in binding buffer), and 130 μL of binding buffer at room temperature for one hour. The incubated binding reaction mixture was then analyzed using HPLC, as described below in Step D.

### Step C—HPLC Analysis of Polysaccharide Standard Binding Reactions and Preparation of Standard Curve

Each of the five ST-5 polysaccharide standard binding reactions prepared as described in Step A (and referred to as ST-5 STD-1 through ST-5 STD-5) were individually analyzed using chromatography condition B (described above in the General Assay Methods section), with an 80 μL injection volume of each binding reaction mixture. Each HPLC analysis was done in duplicate, and the data for each of the five polysaccharide standard binding reactions, are presented in Table 13b.

TABLE 13b

| | Injection | Ps Amt | | | |
|---|---|---|---|---|---|
| ST5 STD | Volume | per INJ | | | Average |
| Binding | (μL) | (μg) | FLR-1 | FLR-2 | FLR |
| ST5 | 80 | 0.0008 | 712038 | 751107 | 731573 |
| STD-1 | | | | | |
| ST5 | 80 | 0.002 | 1812779 | 1919154 | 1865967 |
| STD-2 | | | | | |
| ST5 | 80 | 0.004 | 3697668 | 3838089 | 3767879 |
| STD-3 | | | | | |
| ST5 | 80 | 0.008 | 7465932 | 7738239 | 7602086 |
| STD-4 | | | | | |
| ST5 | 80 | 0.012 | 11306593 | 11722308 | 11514451 |
| STD-5 | | | | | |

| | | |
|---|---|---|
| RSQ | 1.000 | NA |
| Intercept | −62024 | NA |
| Slope | 962390863 | NA |

A five-point standard curve was plotted using the ST-5 amount per injection (ug) as the x-axis, and the average ST-5/anti-ST-5 antibody APC fluorescence peak area as the y-axis. The slope and intercept of this curve were calculated and are provided in Table 12b. The calculated R squared (RSQ) value of 1.0000 indicates good linearity for the curve.

### Step D—HPLC Analysis of Vaccine Antibody Binding Reactions

80 μL samples of the vaccine/antibody binding reaction mixture prepared in Step B were analyzed in duplicate by HPLC using Chromatographic condition B (as described in the General Assay Methods section above). The antibody/polysaccharide complex fluorescence peak area signals were averaged, and presented in Table 13c, along with the amount of ST-5 polysaccharide serotype present in each HPLC injection sample. These data were used as described in Step E to calculate the ST-5 polysaccharide serotype concentration in the PCV15 vaccine drug product.

TABLE 13c

| | | | | | | ST-5 | [Ps] in | Vaccine |
|---|---|---|---|---|---|---|---|---|
| | DP | | | | | amount | Rx | DP |
| Vaccine DP | Prep | Rx Prep | | | Average | per INJ | solution | [ST5] |
| ST5 | Dilution | Dilution | FLR-1 | FLR-2 | FLR | (μg) | (μg/mL) | (μg/mL) |
| Vaccine ST5 | 1.3 | 4 | 6282815 | 6283357 | 6283086 | 0.0066 | 0.082 | 0.43 |

### Step E—Quantification of Free ST-5 in PCV15 Vaccine Drug Product

The average complex peak area provided in Step D, was used along with the slope and intercept of the polysaccharide standard curve from Step C, to calculate the concentration of ST-5 polysaccharide in the vaccine/antibody binding reaction of Step B, using Equations 1a and 1b:

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

This resulted in a calculated value of 0.082 μg/mL for the concentration of free ST-5 polysaccharide in the binding reaction of step D, and a in a calculated value of 0.43 μg/mL for the concentration of free ST-5 polysaccharide in the PCV15 vaccine drug product, as summarized in Table 13d.

TABLE 13d

| | [ST-5] in | Vaccine DP |
|---|---|---|
| Vaccine DP ST-5 | reaction (μg/mL) | [ST-5] (μg/mL) |
| ST-5 vaccine DP | 0.082 | 0.43 |

### Example 14

### Serotype-Specific Quantitation of ST-6A Free Polysaccharide in Multi-Valent Vaccine Drug Product

### Step A—ST-6A Polysaccharide Standard/Anti ST-6A Antibody Binding Reaction

A polysaccharide/anti-polysaccharide antibody binding reaction was carried out by pipetting 2 μL of a ST-6A polysaccharide standard solution (1 μg/mL, standard solution prepared according to the methodology described in Example 1) into 20 μL of anti-ST-6A IgG mAb solution (0.1 mg/mL, solution in binding buffer), and adding additional binding buffer until a total reaction mixture volume of 200 μL was achieved. The resulting binding reaction was then allowed to incubate at room temperature for 1 hour. This reaction was carried out in the same manner five additional times using the same amount of ST-6A IgG mAb solution, and the following amounts of ST-6A polysaccharide standard solution: 5 μL, 10 μL, 20 UL, 30 μL, and 40 L (additional binding buffer was added to each separate binding reaction to achieve a total volume of 200 μL for each reaction). Table 14a summarizes the stoichiometry of each of the five binding reactions.

TABLE 14a

| | 1 μg/mL | 0.1 mg/mL | Binding | Total | Ps | HPLC | | ST6A Amt |
|---|---|---|---|---|---|---|---|---|
| ST6A | 6A Ps STD | anti-ST-6A | Buffer | Vol | Amt | INJ Vol | [ST6A] | Per INJ |
| Binding | (μL) | IgG (μL) | (μL) | (μL) | (μg) | (μL) | (μg/mL) | (μg) |
| ST6A STD-1 | 2 | 20 | 178 | 200 | 0.002 | 100 | 0.01 | 0.005 |
| ST6A STD-2 | 5 | 20 | 175 | 200 | 0.005 | 100 | 0.025 | 0.0125 |
| ST6A STD-3 | 10 | 20 | 170 | 200 | 0.01 | 100 | 0.05 | 0.025 |
| ST6A STD-4 | 20 | 20 | 160 | 200 | 0.02 | 100 | 0.1 | 0.05 |
| ST6A STD-5 | 30 | 20 | 150 | 200 | 0.03 | 100 | 0.15 | 0.075 |
| ST6A STD-6 | 40 | 20 | 140 | 200 | 0.04 | 100 | 0.2 | 0.1 |

### Step B—Vaccine/Anti ST-6A Antibody Binding Reaction

100 μL of a PCV15 vaccine sample stock solution (prepared from a PCV15 vaccine drug product, using the method described in Example 4) was incubated with 20 μL of anti-ST-6A IgG mAb solution (0.1 mg/mL in binding buffer), and 160 μL of binding buffer at room temperature for one hour. The incubated binding reaction mixture was then analyzed using HPLC, as described below in Step D.

### Step C HPLC Analysis of Polysaccharide Standard Binding Reactions and Preparation of Standard Curve

Each of the five ST-6A polysaccharide standard binding reactions prepared as described in Step A (and referred to as ST-6A STD-1 through ST-6A STD-5) were individually analyzed using chromatography condition A (described above in the General Assay Methods section), with an 80 μL injection volume of each binding reaction mixture. Each HPLC analysis was done in duplicate, and the data for each of the five polysaccharide standard binding reactions, are presented in Table 14b.

TABLE 14b

| | | [ST6A] | |
|---|---|---|---|
| Sample | Dilution | (μg/mL) | FLR |
| ST-6A STD-0 | 0 | 0 | 0 |
| ST-6A STD-1 | 100 | 0.01 | 4517677 |
| ST-6A STD-2 | 40 | 0.025 | 11015179 |
| ST-6A STD-3 | 20 | 0.05 | 21673647 |
| ST-6A STD-4 | 10 | 0.1 | 42644274 |
| ST-6A STD-5 | 6.7 | 0.15 | 61546165 |
| ST-6A STD-6 | 5 | 0.2 | 79939300 |

| | | |
|---|---|---|
| RSQ | 0.999 | NA |
| Intercept | 965207 | NA |
| Slope | 401083723 | NA |

A five-point standard curve was plotted using the ST-6A amount per injection (μg) as the x-axis, and the average ST-6A/anti-ST-6A antibody APC fluorescence peak area as the y-axis. The slope and intercept of this curve were calculated and are provided in Table 14b. The calculated R squared (RSQ) value of 0.990 indicates good linearity for the curve.

### Step D—HPLC Analysis of Vaccine/Antibody Binding Reactions

100 μL samples of the vaccine/antibody binding reaction mixture prepared in Step B were analyzed in duplicate by HPLC using Chromatographic condition A (as described in the General Assay Methods section above). The antibody/polysaccharide complex fluorescence peak area signals were averaged, and presented in Table 14c, along with the amount of ST-6A polysaccharide serotype present in each HPLC injection sample. These data were used as described in Step E to calculate the ST-6A polysaccharide serotype concentration in the PCV15 vaccine drug product.

TABLE 14c

| Vaccine | DP Prep | Rx Prep | |
|---|---|---|---|
| DP ST-6A | Dilution | Dilution | FLR |
| DP ST-6A | 1.3 | 2 | 18398065 |

### Step E—Quantification of Free ST-6A in PCV15 Vaccine Drug Product

The average complex peak area provided in Step D, was used along with the slope and intercept of the polysaccharide standard curve from Step C, to calculate the concentration of ST-6A polysaccharide in the vaccine/antibody binding reaction of Step B, using Equation 1:

$$[ Vaccine polysaccharide in binding reaction ] = \frac{Vaccine complex peak area - STD intercept}{STD slope} \qquad Equation 1$$

The polysaccharide concentration of the vaccine drug product was then calculated using the polysaccharide concentration in the vaccine/antibody binding reaction of Step B (obtained using Equation 3 above) and a dilution factor, using Equation-2 (as described above in Example 11, Step E)

This resulted in a calculated value of 0.043 μg/mL for the concentration of free ST-6A polysaccharide in the binding reaction of step D, and a in a calculated value of 0.11 μg/mL for the concentration of free ST-6A polysaccharide in the PCV15 vaccine drug product, as summarized in Table 14d.

TABLE 14d

| | [ST-6A] in | Vaccine DP |
|---|---|---|
| Vaccine DP ST-6A | reaction (μg/mL) | [ST-6A] (μg /mL) |
| ST-6A vaccine DP | 0.043 | 0.11 |

### Example 15

### Serotype-Specific Quantitation of ST-7F Free Polysaccharide in Multi-Valent Vaccine Drug Product

### Step A—ST-7F Polysaccharide Standard/Anti ST-7F Antibody Binding Reaction

A polysaccharide/anti-polysaccharide antibody binding reaction was carried out by pipetting 2 μL of a ST-7F polysaccharide standard solution (1 μg/mL, standard solution prepared according to the methodology described in Example 1) into 20 μL of anti-ST-7F IgG mAb solution (0.1 mg/ml, solution in binding buffer), and adding additional binding buffer until a total reaction mixture volume of 200 μL was achieved. The resulting binding reaction was then allowed to incubate at room temperature for 2 hours. This reaction was carried out in the same manner four additional times using the same amount of ST-7F IgG mAb solution, and the following amounts of ST-7F polysaccharide standard solution: 5 μL, 10 μL, 20 μL, and 30 μL (additional binding buffer was added to each separate binding reaction to achieve a total volume of 200 μL for each reaction). Table 15a summarizes the stoichiometry of each of the five binding reactions.

TABLE 15a

| | 1 μg/mL | 0.05 mg/mL | Binding | Total | Ps |
|---|---|---|---|---|---|
| ST7F STD | ST7F | anti-ST7F | Buffer | Vol | Amt |
| binding | STD (μL) | IgG (μL) | (μL) | (μL) | (μg) |
| ST7F-APC-1 | 2 | 20 | 178 | 200 | 0.002 |
| ST7F-APC-2 | 5 | 20 | 175 | 200 | 0.005 |
| ST7F-APC-3 | 10 | 20 | 170 | 200 | 0.010 |
| ST7F-APC-4 | 20 | 20 | 160 | 200 | 0.020 |
| ST7F-APC-5 | 30 | 20 | 150 | 200 | 0.030 |

### Step B—Vaccine/Anti ST-7F Antibody Binding Reaction

180 μL of a PCV15 vaccine sample stock solution (prepared from a PCV15 vaccine drug product, using the method described in Example 4) was incubated with 20 μL of anti-ST-7F IgG mAb solution (0.05 mg/mL in binding buffer) for two hours. The incubated binding reaction mixture was then analyzed using HPLC, as described below in Step D.

### Step C—HPLC Analysis of Polysaccharide Standard Binding Reactions and Preparation of Standard Curve

Each of the five ST-7F polysaccharide standard binding reactions prepared as described in Step A (and referred to as ST-7F STD-1 through ST-7F STD-5) were individually analyzed using chromatography condition A (described above in the General Methods section), with an 80 μL injection volume of each binding reaction mixture. Each HPLC analysis was done in duplicate, and the data for each of the five polysaccharide standard binding reactions, are presented in Table 15b.

TABLE 15b

| | Injection | Ps Amt | | | |
|---|---|---|---|---|---|
| ST7F | Volume | per INJ | | | Average |
| binding | (μL) | (μg) | FLR-1 | FLR-2 | FLR |
| ST7F | 80 | 0.001 | 786271 | 735642 | 580957 |
| STD-1 | | | | | |
| ST7F | 80 | 0.002 | 1527902 | 1489556 | 1322588 |
| STD-2 | | | | | |
| ST7F | 80 | 0.004 | 2781574 | 2710654 | 2576260 |
| STD-3 | | | | | |
| ST7F | 80 | 0.008 | 4895570 | 4879521 | 4690256 |
| STD-4 | | | | | |
| ST7F | 80 | 0.012 | 7228480 | 7081459 | 7023166 |
| STD-5 | | | | | |

| | | |
|---|---|---|
| RSQ | 0.9985 | NA |
| Inter- | 130543 | NA |
| cept | | |
| Slope | 575574365 | NA |

A five-point standard curve was plotted using the ST-7F amount per injection (μg) as the x-axis, and the average ST-7F/anti-ST-7F antibody APC fluorescence peak area as the y-axis. The slope and intercept of this curve were calculated and are provided in Table 15b. The calculated R squared (RSQ) value of 0.9985 indicates good linearity for the curve.

### Step D—HPLC Analysis of Vaccine/Antibody Binding Reactions

80 μL samples of the vaccine/antibody binding reaction mixture prepared in Step B were analyzed in duplicate by HPLC using Chromatographic condition A (as described in the General Assay Methods section above). The antibody/polysaccharide complex fluorescence peak area signals were averaged, and presented in Table 15c, along with the amount of ST-7F polysaccharide serotype present in each HPLC injection sample. These data were used as described in Step E to calculate the ST-7F polysaccharide serotype concentration in the PCV15 vaccine drug product.

TABLE 15c

| Vaccine | DP Prep | Rx Prep | Inj Vol | | | | ST-7F amt |
|---|---|---|---|---|---|---|---|
| DP ST7F | Dilution | Dilution | (μL) | FLR-1 | FLR-2 | Avg FLR | per INJ (μg) |
| Vaccine | 1.3 | 1.1 | 80 | 3832026 | 3915661 | 3617201 | 0.0061 |
| DP ST7F | | | | | | | |

### Step E—Quantification of Free ST-7F in PCV15 Vaccine Drug Product

The average complex peak area provided in Step D, was used along with the slope and intercept of the polysaccharide standard curve from Step C, to calculate the concentration of ST-7F polysaccharide in the vaccine/antibody binding reaction of Step B, using Equations 1a and 1b:

$$Vaccine Ps Amt per injection ( µg ) = \frac{Sample peak area - STD intercept}{STD slope} \qquad Equation - 1 a$$

$$[ Vaccine Ps in binding reaction ] ( µg / mL ) = ( Ps Amt per injection ) / ( injection volume ) \qquad Equation - 1 b$$

This resulted in a calculated value of 0.076 μg/mL for the concentration of free ST-7F polysaccharide in the binding reaction of step D, and a in a calculated value of 0.11 μg/mL for the concentration of free ST-7F polysaccharide in the PCV15 vaccine drug product, as summarized in Table 15d.

TABLE 15d

| | [ST-7F] in | Vaccine DP |
|---|---|---|
| Vaccine DP ST-7F | reaction (μg/mL) | [ST-7F] (μg/mL) |
| ST-7F DP | 0.076 | 0.11 |

### Example 16

### Serotype-Specific Quantitation of ST-9V Free Polysaccharide in Multi-Valent Vaccine Drug Product

### Step A—ST-9V Polysaccharide Standard/Anti ST-9V Antibody Binding Reaction

A polysaccharide/antibody binding reaction was carried out by pipetting 90 μL of a ST-9V polysaccharide standard solution (1 μg/mL, standard solution prepared according to the methodology described in Example 1) into 60 μL of anti-ST-9V IgG mAb solution (0.1 mg/mL, solution in binding buffer), and adding 450 mL additional binding buffer. The resulting binding reaction was then allowed to incubate at room temperature for 1 hour. Table 16a summarizes the stoichiometry of the binding reaction.

TABLE 16a

| | 1 μg/mL | 0.1 mg/mL | Binding | Total |
|---|---|---|---|---|
| | ST9V | anti-ST9V | Buffer | Vol |
| Complex | STD (μL) | IgG (μL) | (μL) | (μL) |
| ST9V STD Complex | 90 | 60 | 450 | 600 |

### Step B—Vaccine/Anti ST-9V Antibody Binding Reaction

100 μL of a PCV15 vaccine sample stock solution (prepared from a PCV15 vaccine drug product, using the methods described in Example 4) was incubated with 20 μL of anti-ST-9V IgG mAb solution (0.1 mg/mL in binding buffer), and 80 μL of binding buffer at room temperature for one hour. The incubated antibody-vaccine binding reaction mixture was then analyzed using HPLC, as described below in Step D.

### Step C—HPLC Analysis of Polysaccharide Standard Binding Reactions and Preparation of Standard Curve

The single ST-9V polysaccharide standard binding reaction prepared as described in Step A was individually analyzed at different injection volumes using chromatography condition B (described above in the General Assay Methods section, using a 100 μL injection volume of each binding reaction mixture). The volume for each of the five injections are provided below in the second column of Table 16b. The ST-9V Polysaccharide fluorescence peak areas for each corresponding ST-9V injections are shown in the third column of Table 16b.

TABLE 16b

| ST9V STD | Inj Vol (μL) | Ps per INJ (μg) | FLR |
|---|---|---|---|
| ST-9V STD-1 | 10 | 0.0015 | 2330385 |
| ST-9V STD-2 | 25 | 0.00375 | 5723354 |
| ST-9V STD-3 | 50 | 0.0075 | 11473407 |
| ST-9V STD-4 | 75 | 0.01125 | 17211019 |
| ST-9V STD-5 | 100 | 0.015 | 22800523 |

| | | |
|---|---|---|
| RSQ | 1.00 | NA |
| Intercept | 54811 | NA |
| Slope | 1519606003 | NA |

A five-point standard curve was plotted using the ST-9V concentration (in ug/mL) as the x-axis, and the ST-9V/anti-ST-9V antibody APC fluorescence peak area as the y-axis. The slope and intercept of this curve were calculated and are set forth in Table 16b. A calculated R squared (RSQ) value of 1.000 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 16c) was then used in Step E.

TABLE 16c

| | | | DP | | |
|---|---|---|---|---|---|
| | | Vaccine/ | Sample | Vaccine | Ps Amt |
| Vaccine DP | DP | antibody | Vol | complex peak | per Inj |
| ST9V | Dilution | dilution | (μL) | area (FLR) | (μg) |
| DP ST9V | 1.3 | 2 | 80 | 6405171 | 0.0042 |

### Step E—Quantification of Free ST-9V in PCV15 Vaccine Drug Product

The complex peak area provided in Step D, was used along with the slope and intercept of the polysaccharide standard curve from Step C, to calculate the concentration of ST-9V polysaccharide in the vaccine/antibody binding reaction of Step B, using Equations 1a and 1b:

$$Vaccine Ps Amt per injection ( μg ) = \frac{Sample peak area - STD intercept}{STD slope} \qquad Equation - 1 a$$

$$[ Vaccine Ps in binding reaction ] ( μg / mL ) = ( Ps Amt per injection ) / ( injection volume ) \qquad Equation - 1 b$$

This resulted in a calculated value for the concentration of free ST-9V polysaccharide in the PCV15 vaccine drug product of 0.14 μg/mL, as summarized in Table 16d below.

TABLE 16d

| | [ST-9V] in | [ST-9V] (μg/mL) |
|---|---|---|
| | binding | in PCV15 |
| | reaction | vaccine drug |
| Vaccine DP ST-9V | (μg/mL) | product |
| Vaccine DP ST-9V | 0.052 | 0.14 |

### Example 17

### Serotype-Specific Quantitation of ST-14 Free Polysaccharide in Multi-Valent Vaccine Drug Product

### Step A—ST-14 Polysaccharide Standard/Anti ST-14 Antibody Binding Reaction

A polysaccharide/antibody binding reaction was carried out by pipetting 6 μL of a ST-14 polysaccharide standard solution (1 μg/mL, standard solution prepared according to the methodology described in Example 1) into 60 μL of anti-ST-14 IgG mAb solution (0.1 mg/mL, solution in binding buffer), and adding additional binding buffer until a total reaction mixture volume of 200 μL was achieved. The resulting binding reaction was then allowed to incubate at room temperature for 2 hours. This reaction was carried out in the same manner four additional times using the same amount of ST-14 IgG mAb solution, and the following amounts of ST-14 polysaccharide standard solution: 15 μL, 30 μL, 60 μL, and 90 μL (additional binding buffer was added to each separate binding reaction to achieve a total volume of 600 μL for each reaction).

Table 17a summarizes the stoichiometry of each of the five binding reactions.

TABLE 17a

| | 1 μg/mL | 0.1 mg/mL | Binding | Total | |
|---|---|---|---|---|---|
| | ST14 Ps | anti-ST14 | Buffer | Vol | [ST14] |
| ST14 STD binding | STD (μL) | IgG (μL) | (μL) | (μL) | (μg/mL) |
| ST14-STD-1 | 6 | 60 | 534 | 600 | 0.01 |
| ST14-STD-2 | 15 | 60 | 525 | 600 | 0.025 |
| ST14-STD-3 | 30 | 60 | 510 | 600 | 0.05 |
| ST14-STD-4 | 60 | 60 | 480 | 600 | 0.1 |
| ST14-STD-5 | 90 | 60 | 450 | 600 | 0.15 |

### Step B—Vaccine Anti ST-14 Antibody Binding Reaction

300 μL of a PCV15 vaccine sample stock solution (prepared from a PCV15 vaccine drug product, using the methods described in Example 4) was incubated with 60 μL of anti-ST-14 IgG mAb solution (0.1 mg/mL in binding buffer), and 240 μL of binding buffer at room temperature for two hours. The incubated antibody-vaccine binding reaction mixture was then analyzed using HPLC, as described below in Step D.

### Step C—HPLC Analysis of Polysaccharide Standard Binding Reactions and Preparation of Standard Curve

Each of the five ST-14 polysaccharide standard binding reactions prepared as described in Step A was individually analyzed using chromatography condition B (described above in the General Assay Methods section, using an 80 μL injection volume of each binding reaction mixture). The ST-14 serotype for each of the five binding reactions are shown below in the second column of Table 17b. The ST-14 Polysaccharide fluorescence peak areas for each corresponding ST-14 concentration are shown in the third column of Table 17b.

TABLE 17b

| ST14 STD | [ST14] (μg/mL) | FLR peak area |
|---|---|---|
| ST14-STD-1 | 0.01 | 1289431 |
| ST14-STD-2 | 0.025 | 2919343 |
| ST14-STD-3 | 0.05 | 5855623 |
| ST14-STD-4 | 0.1 | 11726570 |
| ST14-STD-5 | 0.15 | 17510202 |
| RSQ (R²) | 1.00 | NA |
| Intercept | 67105 | NA |
| Slope | 116315362 | NA |

A five-point standard curve was plotted using the ST-14 concentration (in ug/mL) as the x-axis, and the ST14/anti-ST-14 antibody APC fluorescence peak area as the y-axis. The slope and intercept of this curve were calculated and are set forth in Table 17b. A calculated R squared (RSQ) value of 1.00 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 17c) was then used in Step E.

TABLE 17c

| | mAb-Ps | [ST14] in | Dilution | | [ST14] |
|---|---|---|---|---|---|
| | Complex | binding | in | Dilution in | (μg/mL) in |
| | FLR peak | reaction | binding | sample | vaccine |
| Sample | area | (μg/mL) | reaction | preparation | DP |
| Vaccine drug | 5349322 | 0.045 | 2 | 1.3 | 0.194 |
| product (ST | | | | | |
| 14 binding) | | | | | |

### Step E—Quantification of Free ST-14 in PCV15 Vaccine Drug Product

The complex peak area provided in Step D, was used along with the slope and intercept of the polysaccharide standard curve from Step C, to calculate the concentration of ST-14 polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 17c above).

This resulted in a calculated value for the concentration of free ST-14 polysaccharide in the PCV15 vaccine drug product of 0.194 μg/mL, as summarized in Table 17d below.

TABLE 17d

| | [ST-14] in | [ST-14] (μg/mL) |
|---|---|---|
| | binding | in PCV15 |
| | reaction | vaccine drug |
| Vaccine DP ST-14 | (μg/mL) | product |
| Vaccine DP ST-14 | 0.045 | 0.194 |

### Example 18

### Serotype-Specific Quantitation of ST-18C Free Polysaccharide in Multi-Valent Vaccine Drug Product

### Step A—ST-18C Polysaccharide Standard/Anti ST-18C Antibody Binding Reaction

A polysaccharide/antibody binding reaction was carried out by pipetting 6 μL of a ST-18C polysaccharide standard solution (1 μg/mL, standard solution prepared according to the methodology described in Example 1) into 20 μL of anti-ST-18C IgG mAb (SEQ ID No. 10) solution (0.1 mg/ml, solution in binding buffer), and adding additional binding buffer until a total reaction mixture volume of 200 μL was achieved. The resulting binding reaction was then allowed to incubate at room temperature for two hours. This reaction was carried out in the same manner four additional times using the same amount of ST-18C IgG mAb solution, and the following amounts of ST-18C polysaccharide standard solution: 15 μL, 30 μL, 60 μL, and 90 μL (additional binding buffer was added to each separate binding reaction as set forth in Table 18a below). Table 18a summarizes the stoichiometry of each of the five binding reactions.

TABLE 18a

| | 1 μg/mL | 0.1 mg/mL | Binding | Total | |
|---|---|---|---|---|---|
| | ST18C Ps | anti-ST18C | Buffer | Vol | [ST18c] |
| ST18C STD binding | STD (μL) | IgG (μL) | (μL) | (μL) | (μg/mL) |
| ST18C-STD-1 | 6 | 60 | 534 | 600 | 0.01 |
| ST18C-STD-2 | 15 | 60 | 525 | 600 | 0.025 |
| ST18C-STD-3 | 30 | 60 | 510 | 600 | 0.05 |
| ST18C-STD-4 | 60 | 60 | 480 | 600 | 0.1 |
| ST18C-STD-5 | 90 | 60 | 450 | 600 | 0.15 |

### Step B—Vaccine Anti ST-18C Antibody Binding Reaction

300 μL of a PCV15 vaccine sample stock solution (prepared from a PCV15 vaccine drug product, using the methods described in Example 4) was incubated with 60 μL of anti-ST-18C IgG mAb solution (0.1 mg/mL in binding buffer), and 240 μL of binding buffer at room temperature for two hours. The incubated antibody-vaccine binding reaction mixture was then analyzed using HPLC, as described below in Step D.

Step C—HPLC Analysis of Polysaccharide Standard Binding Reactions and Preparation of standard Curve

Each of the five ST-18C polysaccharide standard binding reactions prepared as described in Step A was individually analyzed using chromatography condition B (described above in the General Assay Methods section, using an 80 μL injection volume of each binding reaction mixture). The ST-18C serotype for each of the five binding reactions are shown below in the second column of Table 18b. The ST-18C polysaccharide fluorescence peak areas for each corresponding ST-18C concentration are shown in the third column of Table 18b.

TABLE 18b

| | [ST18C] | |
|---|---|---|
| ST18C STD | (μg/mL) | FLR peak area |
| ST18C-STD-1 | 0.01 | 986298 |
| ST18C-STD-2 | 0.025 | 2153600 |
| ST18C-STD-3 | 0.05 | 4168948 |
| ST18C-STD-4 | 0.1 | 7936714 |
| ST18C-STD-5 | 0.15 | 11506201 |
| RSQ (R²) | 0.9995 | NA |
| Intercept | 308280 | NA |
| Slope | 75254815 | NA |

A five-point standard curve was plotted using the ST-18C concentration (in μg/mL) as the x-axis, and the ST-18C/anti-ST-18C antibody APC fluorescence peak area as the y-axis. The slope and intercept of this curve were calculated and are set forth in Table 18b. A calculated R squared (RSQ) value of 0.9995 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 18c) was then used in Step E.

TABLE 18c

| | | [ST18C] in | Dilution | |
|---|---|---|---|---|
| | | binding | in | Dilution in |
| | FLR peak | reaction | binding | sample |
| Vaccine DP ST18C | area | (μg/mL) | reaction | preparation |
| Vaccine DP ST18C | 2295019 | 0.026 | 2 | 1.3 |

### Step E—Quantification of Free ST-18C in PCV15 Vaccine Drug Product

The complex peak area provided in Step D, was used along with the slope and intercept of the polysaccharide standard curve from Step C, to calculate the concentration of ST-18C polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 18c above).

This resulted in a calculated value for the concentration of free ST-18C polysaccharide in the PCV15 vaccine drug product of 0.069 μg/mL, as summarized in Table 18d below.

TABLE 18d

| | \|ST-18C\| in | |
|---|---|---|
| | binding | [ST-18C] (μg/mL) |
| | reaction | in PCV15 vaccine |
| Vaccine DP ST-18C | (μg/mL) | drug product |
| Vaccine DP ST-18C | 0.026 | 0.069 |

### Example 19

### Serotype-Specific Quantitation of ST-19A Free Polysaccharide in Multi-Valent Vaccine Drug Product

### Step A—ST-19A Polysaccharide Standard Anti ST-19A Antibody Binding Reaction

A polysaccharide/antibody binding reaction was carried out by pipetting 2 μL of a ST-19A polysaccharide standard solution (1 μg/mL, standard solution prepared according to the methodology described in Example 1) into 20 μL of anti-ST-19A IgG mAb solution (0.15 mg/ml, solution in binding buffer), and adding additional binding buffer, as indicated in Table 19a below. The resulting binding reaction was then allowed to incubate at room temperature for 1 hour. This reaction was carried out in the same manner four additional times using the same amount of ST-19A IgG mAb solution, and the following amounts of ST-19A polysaccharide standard solution: 5 μL, 30 μL, 20 μL, and 30 μL. Table 19a summarizes the stoichiometry of each of the five binding reactions.

TABLE 19a

| | 1 μg/mL | 0.15 mg/mL | Binding | Total | |
|---|---|---|---|---|---|
| ST19A STD | ST19A | anti-ST19A | Buffer | Vol | [ST19A] |
| binding | STD (μL) | IgG (μL) | (μL) | (μL) | (μg/mL) |
| ST19A-STD-1 | 2 | 20 | 178 | 200 | 0.01 |
| ST19A-STD-2 | 5 | 20 | 175 | 200 | 0.025 |
| ST19A-STD-3 | 30 | 60 | 510 | 600 | 0.05 |
| ST19A-STD-4 | 20 | 20 | 160 | 200 | 0.1 |
| ST19A-STD-5 | 30 | 20 | 150 | 200 | 0.15 |

### Step B—Vaccine/Anti ST-19A Antibody Binding Reaction

45 μL of a PCV15 vaccine sample stock solution (prepared from a PCV15 vaccine drug product, using the methods described in Example 4) was incubated with 30 μL of anti-ST-19A IgG mAb solution (0.15 mg/mL in binding buffer), and 225 μL of binding buffer at room temperature for 1 hour. The incubated antibody-vaccine binding reaction mixture was then analyzed using HPLC, as described below in Step D.

### Step C—HPLC Analysis of Polysaccharide Standard Binding Reactions and Preparation of Standard Curve

Each of the five ST-19A polysaccharide standard binding reactions prepared as described in Step A was individually analyzed using chromatography condition B (described above in the General Assay Methods section, using an 80 μL injection volume of each binding reaction mixture). The ST-19A serotype for each of the five binding reactions are shown below in the second column of Table 19b. The ST-19A Polysaccharide fluorescence peak areas for each corresponding ST-19A concentration are shown in the third column of Table 19b.

TABLE 19b

| | [ST19A] | |
|---|---|---|
| ST19A STD | (μg/mL) | FLR peak area |
| ST19A-STD-1 | 0.01 | 2075026 |
| ST19A-STD-2 | 0.025 | 5102051 |
| ST19A-STD-3 | 0.05 | 10633266 |
| ST19A-STD-4 | 0.1 | 20757756 |
| ST19A-STD-5 | 0.15 | 30290620 |
| RSQ (R²) | 0.9994 | NA |
| Intercept | 221401 | NA |
| Slope | 2528049004 | NA |

A five-point standard curve was plotted using the ST-19A concentration (in ug/mL) as the x-axis, and the ST-19A/anti-ST-19A antibody APC fluorescence peak area as the y-axis. The slope and intercept of this curve were calculated and are set forth in Table 19b. A calculated R squared (RSQ) value of 0.9994 indicates good linearity.

### Step D—HPLC Analysis of Vaccine/Antibody Binding Reactions

45 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 19c) was then used in Step E.

TABLE 19c

| | | [ST-19A] | Dilution | |
|---|---|---|---|---|
| | mAb-Ps | in binding | in | Dilution in |
| | Complex FLR | reaction | binding | sample |
| Sample | peak area | (μg/mL) | reaction | preparation |
| Vaccine DP ST-19A | 17823827 | 0.087 | 6.67 | 1.3 |

### Step E—Quantification of Free ST-19A in PCV15 Vaccine Drug Product

The complex peak area provided in Step D, was used along with the slope and intercept of the polysaccharide standard curve from Step C, to calculate the concentration of ST-19A polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 19c above).

This resulted in a calculated value for the concentration of free ST-19A polysaccharide in the PCV15 vaccine drug product of 0.755 μg/mL, as summarized in Table 19d below.

TABLE 19d

| | [ST-19A] | |
|---|---|---|
| | in binding | [ST-19A] (μg/mL) |
| | reaction | in PCV15 vaccine |
| Vaccine DP ST-19A | (μg/mL) | drug product |
| Vaccine DP ST-19A | 0.087 | 0.755 |

### Example 20

### Serotype-Specific Quantitation of ST-19F Free Polysaccharide in Multi-Valent Vaccine Drug Product

### Step A—ST-19F Polysaccharide Standard/Anti ST-19F Antibody Binding Reaction

A polysaccharide/antibody binding reaction was carried out by pipetting 2 μL of a ST-19F polysaccharide standard solution (1 μg/mL, standard solution prepared according to the methodology described in Example 1) into 20 μL of anti-ST-19F IgG mAb solution (0.15 mg/ml, solution in binding buffer), and adding additional binding buffer, as indicated in Table 20a below. The resulting binding reaction was then allowed to incubate at room temperature for 2 hours. This reaction was carried out in the same manner four additional times using the same amount of ST-19F IgG mAb solution, and the following amounts of ST-19F polysaccharide standard solution: 5 μL, 10 μL, 20 μL, and 30 μL. Table 20a summarizes the stoichiometry of each of the five binding reactions.

TABLE 20a

| | 1 μg/mL | 0.15 mg/mL | Binding | Total | |
|---|---|---|---|---|---|
| ST19F STD | ST19F | anti-ST19F | Buffer | Vol | [ST19F] |
| binding | STD (μL) | IgG (μL) | (μL) | (μL) | (μg/mL) |
| ST19F-STD-1 | 2 | 20 | 178 | 200 | 0.01 |
| ST19F-STD-2 | 5 | 20 | 175 | 200 | 0.025 |
| ST19F-STD-3 | 10 | 20 | 170 | 200 | 0.05 |
| ST19F-STD-4 | 20 | 20 | 160 | 200 | 0.1 |
| ST19F-STD-5 | 30 | 20 | 150 | 200 | 0.15 |

### Step B—Vaccine/Anti ST-19F Antibody Binding Reaction

150 μL of a PCV15 vaccine sample stock solution (prepared from a PCV15 vaccine drug product, using the methods described in Example 4) was incubated with 30 μL of anti-ST-19F IgG mAb solution (0.15 mg/mL in binding buffer), and 120 μL of binding buffer at room temperature for two hours. The incubated antibody-vaccine binding reaction mixture was then analyzed using HPLC, as described below in Step D.

Step C—HPLC Analysis of Polysaccharide Standard Binding Reactions and Preparation of standard Curve

Each of the five ST-19F polysaccharide standard binding reactions prepared as described in Step A was individually analyzed using chromatography condition B (described above in the General Assay Methods section, using an 80 μL injection volume of each binding reaction mixture). The ST-19F serotype for each of the five binding reactions are shown below in the second column of Table 20b. The ST-19F polysaccharide fluorescence peak areas for each corresponding ST-19F concentration are shown in the third column of Table 20b.

TABLE 20b

| | [ST19F] | |
|---|---|---|
| ST19F STD | (μg/mL) | FLR peak area |
| ST19F-STD-1 | 0.01 | 232398 |
| ST19F-STD-2 | 0.025 | 602235 |
| ST19F-STD-3 | 0.05 | 1158384 |
| ST19F-STD-4 | 0.1 | 2351745 |
| ST19F-STD-5 | 0.15 | 3541387 |
| RSQ (R²) | 0.9999 | NA |
| Intercept | −3474 | |
| Slope | 23592592 | |

A five-point standard curve was plotted using the ST-19F concentration (in ug/mL) as the x-axis, and the ST-19F/anti-ST-19F antibody APC fluorescence peak area as the y-axis. The slope and intercept of this curve were calculated and are set forth in Table 20b. A calculated R squared (RSQ) value of 0.9999 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 20c) was then used in Step E.

TABLE 20c

| | mAb-Ps | ST-19F [Ps] | | |
|---|---|---|---|---|
| | Complex | in binding | Dilution in | Dilution in |
| Vaccine DP | FLR peak | reaction | binding | sample |
| ST19F | area | (μg/mL) | reaction | preparation |
| Vaccine DP | 1760820 | 0.075 | 2 | 1.3 |
| ST19F | | | | |

### Step E—Quantification of Free ST-19F in PCV15 Vaccine Drug Product

The complex peak area provided in Step D, was used along with the slope and intercept of the polysaccharide standard curve from Step C, to calculate the concentration of ST-19F polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 20c above).

This resulted in a calculated value for the concentration of free ST-19F polysaccharide in the PCV15 vaccine drug product of 0.194 μg/mL, as summarized in Table 20d below.

TABLE 20d

| | [ST-19F] in | [ST-19F] (μg/mL) in |
|---|---|---|
| Vaccine DP | binding reaction | PCV15 vaccine |
| ST-19F | (μg/mL) | drug product |
| Vaccine DP | 0.075 | 0.194 |
| ST-19F | | |

### Example 21

### Serotype-Specific Quantitation of ST-22F Free Polysaccharide in Multi-Valent Vaccine Drug Product

### Step A—ST-22F Polysaccharide Standard Anti ST-22F Antibody Binding Reaction

A polysaccharide/antibody binding reaction was carried out by pipetting 2 μL of a ST-22F polysaccharide standard solution (1 μg/mL, standard solution prepared according to the methodology described in Example 1) into 20 μL of anti-ST-22F IgG mAb solution (0.15 mg/mL, solution in binding buffer), and adding additional binding buffer, as indicated in Table 21a below. The resulting binding reaction was then allowed to incubate at room temperature for 1 hour. This reaction was carried out in the same manner three additional times using the same amount of ST-22F IgG mAb solution, and the following amounts of ST-22F polysaccharide standard solution: 5 μL, 10 μL, and 20 μL. Table 21a summarizes the stoichiometry of each of the four binding reactions.

TABLE 21a

| | 1 μg/mL | 0.15 mg/mL | Binding | Total | |
|---|---|---|---|---|---|
| ST22F STD | ST22F | anti-ST22F | Buffer | Vol | [ST22F] |
| binding | STD (μL) | IgG (μL) | (μL) | (μL) | (μg/mL) |
| ST22F-STD-1 | 2 | 20 | 178 | 200 | 0.01 |
| ST22F-STD-2 | 5 | 20 | 175 | 200 | 0.025 |
| ST22F-STD-3 | 10 | 20 | 170 | 200 | 0.05 |
| ST22F-STD-4 | 20 | 20 | 160 | 200 | 0.1 |

### Step B—Vaccine/Anti ST-22F Antibody Binding Reaction

150 μL of a PCV15 vaccine sample stock solution (prepared from a PCV15 vaccine drug product, using the methods described in Example 4) was incubated with 30 μL of anti-ST-22F IgG mAb solution (0.15 mg/mL in binding buffer), and 120 μL of binding buffer at room temperature for 1 hour. The incubated antibody-vaccine binding reaction mixture was then analyzed using HPLC, as described below in Step D.

Step C—HPLC Analysis of Polysaccharide Standard Binding Reactions and Preparation of standard Curve

Each of the four ST-22F polysaccharide standard binding reactions prepared as described in Step A was individually analyzed using chromatography condition B (described above in the General Assay Methods section, using an 80 μL injection volume of each binding reaction mixture). The ST-22F serotype for each of the five binding reactions are shown below in the second column of Table 21b. The ST-22F APC fluorescence peak areas for each corresponding ST-22F concentration are shown in the third column of Table 21b.

TABLE 21b

| ST22F STD | [ST22F] (μg/mL) | FLR peak area |
|---|---|---|
| ST22F-STD-1 | 0.01 | 1933968 |
| ST22F-STD-2 | 0.025 | 4657950 |
| ST22F-STD-3 | 0.05 | 9293124 |
| ST22F-STD-4 | 0.1 | 16951409 |
| RSQ (R²) | 0.9976 | NA |
| Intercept | 512918 | |
| Slope | 166404213 | |

A five-point standard curve was plotted using the ST-22F concentration (in ug/mL) as the x-axis, and the ST-22F/anti-ST-22F antibody APC fluorescence peak area as the y-axis. The slope and intercept of this curve were calculated and are set forth in Table 21b. A calculated R squared (RSQ) value of 0.9976 indicates good linearity.

### Step D—HPLC Analysis of Vaccine/Antibody Binding Reactions

80 μL of the vaccine/antibody binding reaction mixture prepared in Step B was analyzed by HPLC using Chromatographic condition B (as described in the General Assay Methods section above). The antibody/polysaccharide complex fluorescence peak area signal generated (see Table 21c) was then used in Step E.

TABLE 21c

| | | ST-22F [Ps] | | |
|---|---|---|---|---|
| | mAb-Ps | in binding | Dilution in | Dilution in |
| Vaccine DP | Complex FLR | reaction | binding | sample |
| ST22F | peak area | (μg/mL) | reaction | preparation |
| Vaccine DP | 8409878 | 0.047 | 2 | 1.3 |
| ST22F | | | | |

### Step E—Quantification of Free ST-22F in PCV15 Vaccine Drug Product

The complex peak area provided in Step D, was used along with the slope and intercept of the polysaccharide standard curve from Step C, to calculate the concentration of ST-22F polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 21c above).

This resulted in a calculated value for the concentration of free ST-22F polysaccharide in the PCV15 vaccine drug product of 0.123 μg/mL, as summarized in Table 21d below.

TABLE 21d

| | [ST-22F] in | [ST-22F] (μg/mL) |
|---|---|---|
| Vaccine DP | binding reaction | in PCV15 vaccine |
| ST-22F | (μg/mL) | drug product |
| Vaccine DP | 0.047 | 0.123 |
| ST-22F | | |

### Example 22

### Serotype-Specific Quantitation of ST-23F Free Polysaccharide in Multi-Valent Vaccine Drug Product

### Step A—ST-23F Polysaccharide Standard/Anti ST-23F Antibody Binding Reaction

A polysaccharide/antibody binding reaction was carried out by pipetting 1 μL of a ST-23F polysaccharide standard solution (1 μg/mL, standard solution prepared according to the methodology described in Example 1) into 20 μL of anti-ST-23F IgG mAb solution (0.1 mg/mL, solution in binding buffer), and adding additional binding buffer, as indicated in Table 22a below. The resulting binding reaction was then allowed to incubate at room temperature for 2 hours. This reaction was carried out in the same manner three additional times using the same amount of ST-23F IgG mAb solution, and the following amounts of ST-23F polysaccharide standard solution: 5 μL, 10 μL, and 20 μL. Table 22a summarizes the stoichiometry of each of the four binding reactions.

TABLE 22a

| | 1 μg/mL | 0.1 mg/mL | | | | |
|---|---|---|---|---|---|---|
| ST23F STD | ST23F STD | anti-ST23F | Binding | Total | Ps Amt Per | [ST23F] |
| binding | (μL) | IgG (μL) | Buffer (μL) | Vol (μL) | INJ (μg) | (μg/mL) |
| ST23F-STD-1 | 2 | 20 | 178 | 200 | 0.0008 | 0.01 |
| ST23F-STD-2 | 5 | 20 | 175 | 200 | 0.002 | 0.025 |
| ST23F-STD-3 | 10 | 20 | 170 | 200 | 0.004 | 0.05 |
| ST23F-STD-4 | 20 | 20 | 160 | 200 | 0.008 | 0.1 |

### Step B—Vaccine/Anti ST-23F Antibody Binding Reaction

150 μL of a PCV15 vaccine sample stock solution (prepared from a PCV15 vaccine drug product, using the methods described in Example 4) was incubated with 30 μL of anti-ST-23F IgG mAb solution (0.1 mg/mL in binding buffer), and 120 μL of binding buffer at room temperature for two hours. The incubated antibody-vaccine binding reaction mixture was then analyzed using HPLC, as described below in Step D.

### Step C HPLC Analysis of Polysaccharide Standard Binding Reactions and Preparation of Standard Curve

Each of the five ST-23F polysaccharide standard binding reactions prepared as described in Step A was individually analyzed using chromatography condition B (described above in the General Assay Methods section, using an 80 μL injection volume of each binding reaction mixture). The ST-23F serotype for each of the five binding reactions are shown below in the second column of Table 22b. The ST-23F Polysaccharide fluorescence peak areas for each corresponding ST-23F concentration are shown in the third column of Table 22b.

TABLE 22b

| ST23F STD | [ST23F] (μg) | FLR peak area |
|---|---|---|
| ST23F-STD-1 | 0.0008 | 2164565 |
| ST23F-STD-2 | 0.002 | 5253017 |
| ST23F-STD-3 | 0.004 | 10743679 |
| ST23F-STD-4 | 0.008 | 21346780 |
| RSQ (R²) | 0.9999 | NA |
| Intercept | 5369 | |
| Slope | 2670913330 | |

A four-point standard curve was plotted using the ST-23F concentration (in ug/mL) as the x-axis, and the ST-23F/anti-ST-23F antibody APC fluorescence peak area as the y-axis. The slope and intercept of this curve were calculated and are set forth in Table 22b. A calculated R squared (RSQ) value of 0.9999 indicates good linearity.

### Step D—HPLC Analysis of Vaccine Antibody Binding Reactions

80 μL of the vaccine/antibody binding reaction mixture prepared in Step B was analyzed by HPLC using Chromatographic condition B (as described in the General Methods section above). The antibody/polysaccharide complex fluorescence peak area signal generated (see Table 22c) was then used in Step E.

TABLE 22c

| | | ST-23F [Ps] | | |
|---|---|---|---|---|
| | | in binding | Dilution in | Dilution in |
| | FLR peak | reaction | binding | sample |
| Vaccine DP ST23F | area | (μg/mL) | reaction | preparation |
| Vaccine DP ST23F | 8599604 | 0.040 | 2 | 1.3 |

### Step E—Quantification of Free ST-23F in PCV15 Vaccine Drug Product

The complex peak area provided in Step D, was used along with the slope and intercept of the polysaccharide standard curve from Step C, to calculate the concentration of ST-23F polysaccharide in the vaccine/antibody binding reaction of Step B, using equation 1:

$$[ Vaccine polysaccharide in binding reaction ] = \frac{Vaccine complex peak area - STD intercept}{STD slope} \qquad Equation - 1$$

The polysaccharide concentration of the vaccine drug product was then calculated using the polysaccharide concentration in the vaccine/antibody binding reaction of Step B and a dilution factor, using Equation-2:

$$[ Vaccine drug product polysaccharide ] = Dilution factor * [ Vaccine Ps in binding reaction ] \qquad Equation - 2$$

wherein the dilution factor in Equation-2 is equal to the dilution in the vaccine/antibody binding reaction of Step B, multiplied by the DP sample preparation dilution factor in Step A (see dilution values in Table 21c above).

This resulted in a calculated value for the concentration of free ST-23F polysaccharide in the PCV15 vaccine drug product of 0.105 μg/mL, as summarized in Table 22d below.

TABLE 22d

| | [ST-23F] in | [ST-23F] (μg/mL) |
|---|---|---|
| Vaccine DP | binding reaction | in PCV15 vaccine |
| ST-23F | (μg/mL) | drug product |
| Vaccine DP | 0.040 | 0.105 |
| ST-23F | | |

### Example 23

### Preparation of FLR Tag Labeled Anti-Serotype Antibody Mixture

Fluorescence (FLR) labeled pneumococcal anti-ST mAbs and anti-CRM197 mAbs can be generated using the methods described, for example, in Chen, et al., BMC Infectious Diseases. 18, 613 (2018) and Cox et al., J. Immunol. 200 (Supp 1), 180 (2018).

An anti-ST-4 IgG mAb was incubated with excess equivalents of Alexa Fluor™ 350 NHS ester PBS buffer for three hours at ambient temperature. The resulting reaction mixture was purified by desalting through a Zeba Spin desalting column (Thermo Fisher), to provide AF350 labeled anti-ST-4 (anti-ST-4-AF350) mAb.

Using the same methodology, the following FLR labeled anti-ST5, anti-ST6A and anti-ST14 mAbs were prepared: anti-ST5-AF430, anti-ST6A-AF555 and anti-ST14-AF633 (made by incubating the antibodies with Alexa Fluor™ 430, Alexa Fluor™ 555 and Alexa Fluor™ 633 NHS buffer, respectively). The four FLR labeled anti-ST mAbs were then mixed together as a cocktail, which contained 0.2 mg/mL of each of the four labeled antibodies, and this cocktail was used to multiplex with a multivalent PCV vaccine product or a PCV vaccine standard.

### Example 24

### Formation of APC in Multiplex Format and Generation of Standard Curve

### Step A—Formation of Polysaccharide Anti-Polysaccharide APC in Multiplex Format

A solution containing four pneumococcal vaccine polysaccharide serotypes (ST-4, ST-5, ST-6A, and ST-14:1 μg/mL each serotype) was complexed with the FLR labeled anti-serotype antibody cocktail prepared in Example 24 (containing 0.2 mg/mL each of fluorescence-labeled anti-ST-4, anti-ST5, anti-ST6A, and anti-ST14 antibodies), using the binding reaction methodology described in Example 9, Step A. The binding reaction was carried out in the same manner five times using the reaction stoichiometry set forth in Table 24a, to prepare 5 separate APCs.

TABLE 24a

| | PS Standard | | | | Each |
|---|---|---|---|---|---|
| | (1 μg/mL | FLR-labeled | Binding | Total | serotype |
| | each ST) | mAb cocktail | Buffer | Vol | [Ps] |
| Standard | (μL) | (μL) | (μL) | (μL) | (μg/mL) |
| STD-1 | 5 | 15 | 180 | 200 | 0.025 |
| STD-2 | 10 | 15 | 175 | 200 | 0.05 |
| STD-3 | 20 | 15 | 165 | 200 | 0.1 |
| STD-4 | 30 | 15 | 155 | 200 | 0.15 |
| STD-5 | 40 | 15 | 145 | 200 | 0.2 |

### Step B—Formation of Vaccine Product/Anti-Polysaccharide APC in Multiplex Format

A PCV15 vaccine drug product (containing adjuvant and 4 mcg/mL of each of the following polysaccharide serotypes: 1, 3, 4, 5, 6A, 6B, 7F, 9V, 14, 18C, 19A, 19F, 22F, 23F and 33F) was diluted 8-fold by binding buffer to a product solution (labeled as “Product-8x” in Table 24b). The resulting diluted mixture was then complexed with the FLR labeled anti-serotype antibody cocktail prepared in Example 24 (containing 0.2 mg/mL each of fluorescence-labeled anti-ST-4, anti-ST5, anti-ST6A, and anti-ST14 antibodies), using the binding reaction methodology described in Example 9, Step B, and using the reaction stoichiometry summarized in Table 24b to provide a vaccine/antibody APC reaction mixture that was directly analyzed using the methods described in Example 25.

TABLE 24b

| | | 0.2 mg/mL ea | Binding | Total |
|---|---|---|---|---|
| | Product-8x | of 4 FLR-mAb | Buffer | Vol |
| Sample | (μL) | Mix (μL) | (μL) | (μL) |
| PCV15 Vaccine | 20 | 15 | 165 | 200 |
| binding reaction | | | | |

### Example 25

### Chromatographic Analysis of the FLR Labeled APCs for Multiplex Binding Reactions

### Step A—HPLC Analysis

All multiplex binding reactions prepared according to Example 24. Steps A and B were analyzed using HPLC (Using either chromatography condition A or B, as described above in the General Assay Methods Section) for quantification by using the APC peak areas. Four fluorescence detection channels were set on the HPLC instrument to detect the FLR signal specific to each of the four FLR labeled antibodies. The four fluorescence detection channels were set as follows: Anti-ST-4-AF350 and its APC are detected at Exciting/Emission (Ex/Em) of 346 nm/442 nm; Anti-ST5-AF430 and its APC are detected at Ex/Em of 433 nm/541 nm: Anti-ST6A-AF555 and its APC are detected on Ex/Em of 555 nm/565 nm: Anti-ST14-AF633 and its APC are detected at Ex/Em of 633 nm/647 nm. Both exciting and emission wavelengths can be slightly varied, and still maintain the signal specificity to the FLR labeled mAb. Chromatograms were produced for the five APCs made in Example 24, Step A and the single APC made in Example 24, Step B.

### Step B—Generation of Standard Curve for Polysaccharide Antibody Complexes

The six total chromatograms produced in Step A were collected and processed on all four FLR detection channels. The peak areas were integrated using Waters Empower 3 software. For each of the four polysaccharide serotypes (ST-4, ST-5, ST-6A, and ST-14) being quantified, the five APC peak areas for each concentration in Step A is shown in Table 25a.

TABLE 25a

| | STD Curve | | | |
|---|---|---|---|---|
| | ST-4 | ST5 | ST6A | ST14 |
| [Ps] (μg/mL) | FLR350 | FLR430 | FLR555 | FLR633 |
| 0.025 | 13862908 | 3271276 | 14629276 | 1191432 |
| 0.05 | 26449246 | 6062365 | 28413513 | 2344125 |
| 0.1 | 48912423 | 11303380 | 54599861 | 4446046 |
| 0.15 | 73535345 | 16928391 | 84152265 | 6757758 |
| 0.2 | 87356619 | 20278356 | 104312971 | 8126979 |
| R² | 0.991 | 0.993 | 0.997 | 0.993 |
| Intercept | 4951848 | 1137529 | 2465360 | 323634 |
| Slope | 429252002 | 99344993 | 521487784 | 40472705 |

A linear standard curve was generated for each of each of the four serotypes using the data provided in Table 25a.

The total polysaccharide concentration (conjugate+free polysaccharide) of each serotype can be calculated out using Equation-1, by the linear fit obtained from each standard curve.

$$[ DP Ps in binding reaction ] = \frac{DP sample peak area - STD intercept}{STD slope} \qquad Equation - 1$$

Wherein the term “DP Ps in binding reaction” refers to the concentration of a particular polysaccharide that was present in the APC made in Example 24, Step B.

### Step C—Analysis of Vaccine/Antibody Binding Reaction

The polysaccharide concentrations generated from Equation-1 (concentrations in binding reaction) were converted to the concentrations in the PCV vaccine product with the product sample preparation dilution factor as described below in Table 25b

TABLE 25b

| | PCV product | | | |
|---|---|---|---|---|
| | ST-4 | ST5 | ST6A | ST14 |
| FLR detection channel | FLR350 | FLR430 | FLR555 | FLR633 |
| APC Peak area | 52581520 | 11054715 | 49747133 | 4163950 |
| Binding Rx [Ps] (μg/mL) | 0.111 | 0.100 | 0.091 | 0.095 |
| Product sample dilution | 80 | 80 | 80 | 80 |
| factor | | | | |
| PCV product total [Ps] | 8.9 | 8.0 | 7.3 | 7.6 |
| (μg/mL) | | | | |

This resulted in a calculated value for the concentration of: total ST-4 polysaccharide in the PCV15 vaccine drug product of 8.9 μg/mL; total ST-5 polysaccharide in the PCV15 vaccine drug product of 8.0 μg/mL; total ST-6A polysaccharide in the PCV15 vaccine drug product of 7.3 g/mL; and the concentration of free ST-14 polysaccharide in the PCV15 vaccine drug product of 7.6 μg/mL; as summarized in Table 25b above. The total polysaccharide concentration is the sum of conjugated polysaccharide and free polysaccharide in the vaccine sample.

These results demonstrate that multiple vaccine polysaccharide serotypes can be simultaneously quantified using a multiplex assay.

1.-20. (canceled)

21. A method for identification and/or quantification of a polysaccharide serotype present in a vaccine drug product, said method comprising the steps:
a) obtaining a standard sample comprising a polysaccharide serotype corresponding to a serotype present in the vaccine drug product;
b) obtaining a vaccine drug product sample stock solution that was prepared from the vaccine drug product;
c) adding to the vaccine drug product sample stock solution, a serospecific anti-polysaccharide antibody corresponding to the polysaccharide serotype of step (a) creating a mixture, wherein the amount of serospecific anti-polysaccharide antibody added is sufficient to ensure that all antibody binding sites on the polysaccharide serotype in the vaccine drug product stock solution are occupied by the corresponding serospecific anti-polysaccharide antibody, to form an antibody-polysaccharide complex (APC) in the mixture;
d) subjecting the APC in the mixture to a chromatographic separation method to provide a quantitative peak area; and
e) using a linear fit equation to calculate the amount of the free polysaccharide of the serotype used in step (a) that is present in the mixture, wherein the linear fit equation takes into account the slope and intercept of a standard curve for the polysaccharide serotype corresponding to the polysaccharide serotype of step (a) along with the quantitative peak area generated in step (d); and
f) optionally repeating steps (a) through (e) one or more times to identify and/or quantify other polysaccharide serotypes that are present in the vaccine drug product.

22. The method of claim 21, wherein the polysaccharide serotype is a S. pneumoniae serotype, and is selected from the group consisting of serotypes 1, 3, 4, 5, 6A, 6B, 7F, 9V, 14, 18C, 19A, 19F, 22F, 23F, and 33F.

23. The method of claim 21, wherein the standard curve for the polysaccharide serotype corresponding to the polysaccharide serotype of step (a) was generated using a method comprising the following steps:
(i) taking one or more aliquots of the standard sample prepared in step (a);
(ii) diluting one aliquot to a known concentration using a buffer, or diluting multiple aliquots to different known concentrations using a buffer;
(iii) adding to the aliquot(s) made in step (ii) the serospecific anti-polysaccharide antibody specific against the polysaccharide serotype of step (a), creating a binding reaction mixture, wherein the amount of serospecific anti-polysaccharide antibody added to each aliquot is sufficient to ensure that all antibody binding sites on the polysaccharide serotype of step (a) in the aliquot are occupied by the corresponding serospecific anti-polysaccharide antibody, then incubating each of the resulting binding reactions for a time and at a temperature sufficient to ensure that all of the polysaccharide serotypes corresponding to the polysaccharide serotypes of step (a) in each aliquot is saturated with its corresponding serospecific anti-polysaccharide antibody;
(iv) subjecting each of the binding reaction mixtures prepared in step (iii) to a chromatographic separation method, wherein said chromatographic separation method allows for the detection and quantification of the antibody-polysaccharide complex that is present in each of the binding reaction mixtures; and
(v) generating a standard curve using data obtained from the chromatographic separations.

24. The method of claim 21, wherein the serospecific anti-polysaccharide antibody used is a modified antibody or an antibody fragment.

25. The method of claim 21, wherein the serospecific anti-polysaccharide antibody used is a fluorescence-labeled antibody.

26. The method of claim 21, wherein the chromatographic separation in step (d) is carried out in a buffered solution at pH of 5 to 9.

27. The method of claim 26, wherein the buffered solution comprises a salt.

28. The method of claim 27 wherein the buffered solution has pH from 6 to 8, and a salt concentration of 0.05M to 1M.

29. The method of claim 21, wherein the chromatographic separation in step d) is carried out using size-exclusion chromatography, ion-exchange chromatography, or capillary electrophoresis.

30. The method of claim 23, wherein the chromatographic separation in part iv) is carried out using size-exclusion chromatography, ion-exchange chromatography, or capillary electrophoresis.

31. The method of claim 29, wherein the chromatographic separation is carried out using a buffered mobile phase.

32. The method of claim 31, wherein the mobile phase is a buffer comprising amino acids.

33. The method of claim 32, wherein the mobile phase is a bis-tris buffer.

34. The method of claim 30, wherein the mobile phase comprises a salt.

35. The method of claim 21 wherein the separation methods to provide a quantitative peak area are detected by fluorescence or ultraviolet light.

36. The method of claim 21, wherein the APCs are detected and quantified using a multiplex assay.

37. The method of claim 35, wherein multiple polysaccharide serotypes are simultaneously detected at different wavelengths.

38. The method of claim 21, wherein the method of claim 21 is used to identify and/or quantify all polysaccharide serotypes present in a vaccine drug product.
